(19) United States
(12) Patent Application Publication
(10) Pub. No.: US 2023/0039858 A1
(43) Pub. Date: Feb. 9, 2023

(54) COMPOSITIONS AND METHODS RELATED TO MOLECULAR CONJUGATION

(71) Applicant: IntoCell, Inc., Daejeon (KR)

(72) Inventors: Taekyo Park, Daejeon (KR); Sunyoung Kim, Daejeon (KR); Suho Park, Daejeon (KR); Doohwan Jung, Daejeon (KR); Donghoon Seo, Daejeon (KR); Sangkwang Lee, Daejeon (KR); Sanghyeon Yun, Daejeon (KR); Jihyeon Ha, Daejeon (KR); Hyang Sook Lee, Daejeon (KR); Okku Park, Daejeon (KR); Beomseok Seo, Daejeon (KR); Sena Kim, Daejeon (KR); Minah SeoI, Daejeon (KR); Jina Song, Daejeon (KR)

(21) Appl. No.: 17/781,467

(22) PCT Filed: Dec. 1, 2020
(86) PCT No.: PCT/IB2020/001003

§ 371 Date: Jun. 1, 2022

Related U.S. Application Data
(60) Provisional application No. 62/942,482, filed on Dec. 2, 2019.

(51) Int. Cl. A61K 47/68; A61K 47/54

(52) U.S. Cl. CPC A61K 47/6849; A61K 47/545; A61K 47/549

(57) ABSTRACT

The invention relates to activated Michael acceptor (AMA) compounds that can undergo conjugation with biomolecules containing Michael donor moieties, thereby providing plasma-stable antibody-drug conjugates (ADCs). Pharmaceutical compositions of the ADCs are disclosed as well. Also provided herein are a number of applications (e.g., therapeutic applications) in which the compositions are useful.

### RELATED APPLICATIONS

This application claims the benefit of priority to U.S. Provisional Patent Application No. 62/942,482, filed Dec. 2, 2019. The entire contents of this application are incorporated herein by reference.

### BACKGROUND

Antibody-drug conjugates (ADCs) are emerging as a powerful class of anti-tumor agents with efficacy across a range of cancers. ADCs commonly include three distinct features: a cell-binding agent or targeting moiety (the antibody); a linker; and a cytotoxic agent (the drug) (FIG. 1). Antibody bioconjugation, therefore, has a crucial role in the development of novel biologically active conjugates for applications in biology and medicine. Chemoselective and mild processes are key for precisely installing modifications without disturbing the structure, function and activity of the antibody. Reactivity, accessibility and abundance of amino acid side chains are key aspects required to achieve selective modification of one certain residue over all other proteinogenic amino acids. Among these, cysteine (Cys) remains the amino acid of choice due to its low abundance and the high nucleophilicity of its sulfhydryl side chain.

Despite the developments in the field of antibody bioconjugation, maleimides are still the most commonly used reagents, mainly due to fast kinetics and easy synthesis of maleimide reagents. However, the thio-succinimidyl conjugates formed from this reagent undergo exchange reactions with thiols present in plasma leading to the release of the maleimide. In the case of ADCs, this can lead to toxicity, as the product of the thiol exchange reaction is a highly potent cytotoxic drug. Thus, there is a pressing need for methods to build protein and antibody conjugates in a manner that allows for site-selective and irreversible installation of probes and drugs at specific sites within their sequence, resulting in plasma-stable ADCs that reliably release drug at the intended target site.

The thiols of interchain cysteine residues in monoclonal antibodies can be used as attachment sites for drug molecules. In a human IgG1, there are four interchain disulfide bonds that can be used as conjugation sites. The four interchain disulfide bonds can be reduced by tris(2-carboxyethyl) phosphine (TCEP) or dithiothreitol (DTT), which results in eight thiol groups that are available for conjugating drug molecules. Through this method, different drug antibody ratio (DAR) conjugates can be obtained.

Classically, cysteine residues can be modified through addition of thiols to electrophiles such as maleimides. The conjugate can thus be prepared by reducing the disulfide bonds of the antibody and then reacting with maleimides. However, maleimide-based antibody-drug conjugates have been found to possess limited stability in blood circulation. Therefore, an alternative method of conjugating of cysteine residues to active moieties, which would result in an ADCs not susceptible to the retro-Michael reactions, is needed.

### SUMMARY OF INVENTION

In certain embodiments, the present disclosure relates to a compound of formula (I):

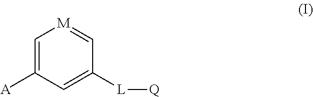

or a salt thereof, wherein:

### A is

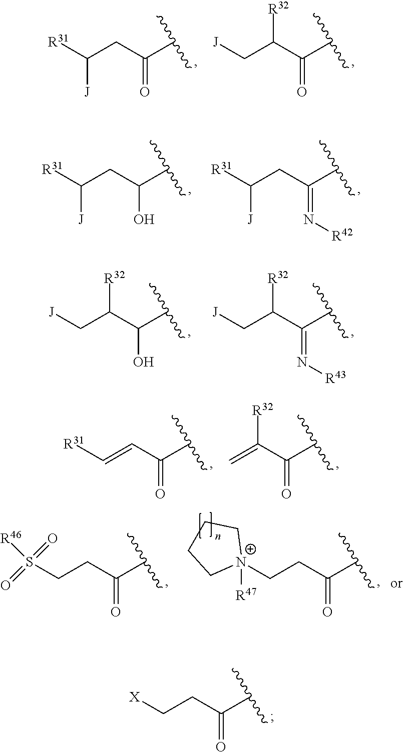

### M is N, CR³⁰, or C(-L-Q);

each L is independently selected from a spacer moiety;
each Q is independently selected from an active moiety or a reactive group;
X is selected from —Cl, —Br, and —I;
J is a targeting moiety;
R³⁰ and R³¹ are each independently selected from an electron-withdrawing group, hydrogen, alkyl, alkenyl, alkynyl, aryl, heteroaryl, carbocyclyl, heterocyclyl, and haloalkyl;
R⁴⁶ is selected from alkyl, alkenyl, alkynyl, aryl, heteroaryl, carbocyclyl, heterocyclyl, and haloalkyl;
R⁴² and R⁴³ are each independently selected from —OH, alkoxy, —NR⁴⁴R⁴⁵ alkyl, alkenyl, alkynyl, aryl, heteroaryl, carbocyclyl, and heterocyclyl, wherein R⁴⁴ and R⁴⁵ together with the nitrogen atom to which they are attached can form a 5-8-membered cycle, optionally fused with an aryl or a heteroaryl ring;
R³², R⁴⁴, and R⁴⁵ are each independently selected from hydrogen, alkyl, alkenyl, alkynyl, aryl, heteroaryl, carbocyclyl, heterocyclyl, and haloalkyl;
R⁴⁷ is O⁻, alkyl, alkenyl, alkynyl, aryl, heteroaryl, carbocyclyl, heterocyclyl; and n is 1 to 4.

In certain embodiments, the present disclosure relates to a method of preparing a conjugate, comprising reacting the compound of formula (I) with a reagent comprising a targeting moiety covalently bound to a Michael donor, thereby producing a Michael adduct.

In certain embodiments, the present disclosure relates to a pharmaceutical composition comprising a compound of formula (I), or a pharmaceutically acceptable salt thereof, and a pharmaceutically acceptable excipient.

In certain embodiments, the present disclosure relates to a method for treating a subject in need thereof, comprising administering a compound of formula (I) or a pharmaceutically acceptable salt thereof, or administering a composition comprising a compound of formula (I), or a pharmaceutically acceptable salt thereof, and a pharmaceutically acceptable excipient.

### BRIEF DESCRIPTION OF THE DRAWINGS

FIG. 1 is a schematic depiction of an antibody-drug conjugate (ADC).

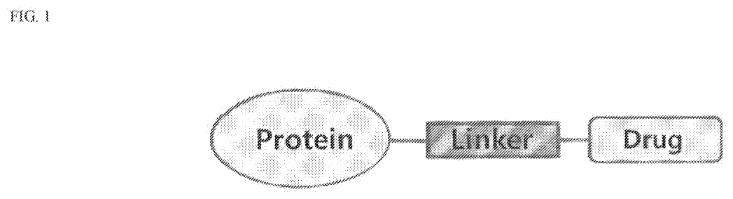

FIG. 2 depicts High Performance Liquid Chromatography (HPLC) graphs demonstrating stability of AMA-9c in aqueous environment over time.

FIG. 3 illustrates relative reaction rates of N-Ac-Cysteine with compounds AMA-1, AMA-2, AMA-3, AMA-4, AMA-8, AMA-9c, AMA-10, mMPS-4, and PyrMPS-1.

FIG. 4 shows the relative stability of compound N-Ac-Cys-AMA-10 compared to compound 14.

FIG. 5 is a reaction scheme depicting conjugation reaction of AMA-9c with human serum albumin (1), and Thio-mAb (2).

FIG. 6 is a hydrophobic interaction chromatography-HPLC (HIC-HPLC) graph depicting analysis of the conjugation reaction of AMA-9c with Thio-mAb.

FIG. 7 is a scheme depicting relative reaction rates of Michael acceptor precursor Reference A compared to AMA precursors pryMPS-1 and mMPS-4 with N-Ac-cysteine.

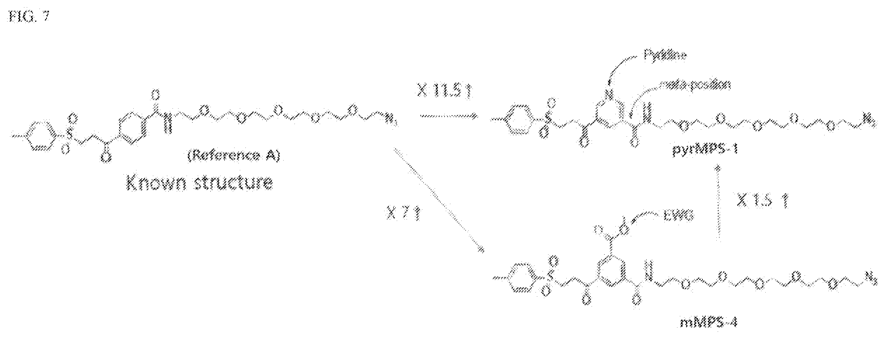

### DETAILED DESCRIPTION OF THE INVENTION

Compounds of the Disclosure

In certain aspects, the present disclosure relates to activated Michael acceptor (AMA) compounds that can undergo conjugation with biomolecules containing Michael donor moieties. AMAs, such as the vinyl aryl ketones of formulas (II) and (III), can undergo Michael addition reactions with a Michael donor to give conjugates of formulas (IIa) and (IIIa) (Scheme 1). In certain aspects, the present disclosure relates to conjugates of biomolecules with AMAs, such as compounds (IIa) and (IIIa).

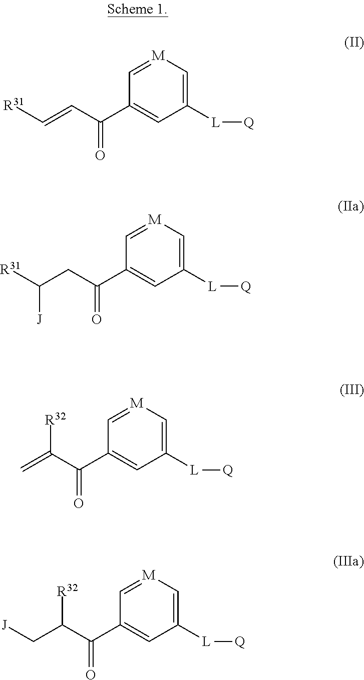

In the compounds of formulas (II), (III), (IIa), and (IIIa), L is a spacer moiety; Q is an active moiety (which may, for example, comprise a drug moiety, as described elsewhere herein) or a reactive group; M is N, CR³⁰, or C(-L-Q); J is a targeting moiety (which may, for example, comprise an antibody, as described elsewhere herein); R³⁰ and R³¹ are each independently selected from an electron-withdrawing group, hydrogen, alkyl, alkenyl, alkynyl, aryl, heteroaryl, carbocyclyl, heterocyclyl, and haloalkyl. For example, in certain embodiments at least one of R³⁰ and R³¹ is present and is an electron-withdrawing group; and R³² is selected from hydrogen, alkyl, alkenyl, alkynyl, aryl, heteroaryl, carbocyclyl, heterocyclyl, and haloalkyl.

In some embodiments of the present invention, the Michael donor is a Cys residue of a protein, such as an antibody, or a fusion protein.

In some embodiments, the active moiety Q comprises L′ and Q′, wherein L′ is a linker and Q′ is an active agent. In some aspects of the invention, L′ comprises a coupling group, wherein the coupling group is coupled to L. For example, the coupling group may be selected from —C(═O)NR³²—, —C(═O)O—, —C(═NR³²)—, —C═NO—, —NR³²—C(═O)—NR³²—, —OC(═O)O—, —S—S—, —NR³²S(═O)₂O—, and —OS(═O)₂O—. Alternatively, the coupling group is selected from:

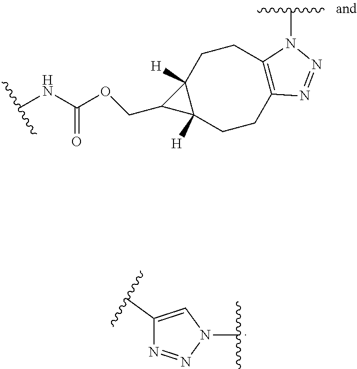 [SMILES: *NC(=O)OCC1[C@H]2CCc3nnn(*)c3CC[C@@H]12 ; *c1cn(*)nn1]

L′ can further comprise a cleavable group, wherein the cleavable group is coupled to Q′. For example, the cleavable group coupled to Q′ may be selected from:

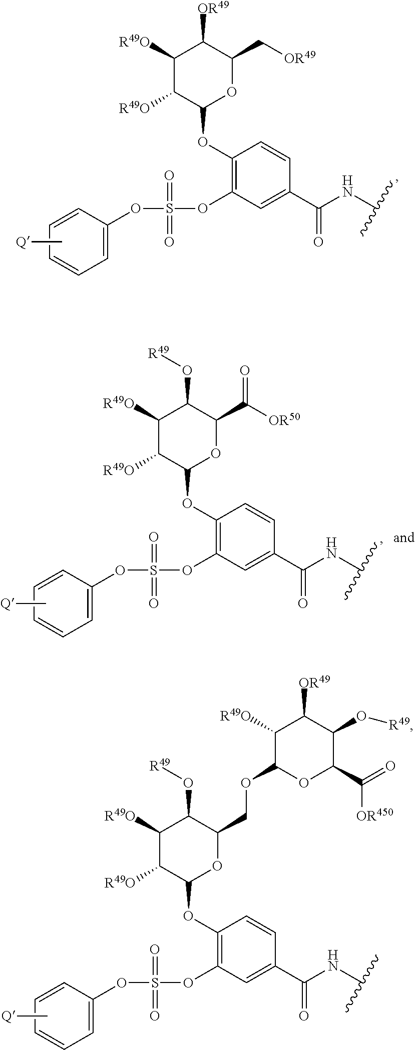

wherein

R⁴⁹ is hydrogen or —C(═O)R⁵⁰; and

R⁵⁰ is lower alkyl.

Additional examples of cleavable groups are disclosed in International Patent Application Publication No. WO2019/008441, which is hereby incorporated by reference in its entirety.

In some embodiments, L′ further comprises a C₆-C₁₀₀ alkylene comprising at least one group selected from —NH—, —C(═O)—, —O—, —S—, —S(O)—, and —S(═O)₂—.

In certain embodiments, the spacer moiety comprises a C₆-C₁₀₀ alkylene comprising at least one group selected from —NH—, —C(═O)—, —O—, —S—, —S(O)—, and —S(═O)₂—.

Additionally or alternatively, the spacer moiety may comprise

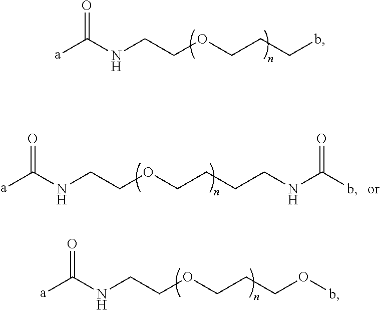

wherein

a is the bond to the M-containing aromatic ring, and b is the bond to L′; and

n is 2-20.

In some embodiments of the invention, Q′ is a hormone, an oligonucleotide, a toxin, an affinity ligand, a probe for detection, or a combination thereof. For example, Q′ may be selected from a cytokine, an immunomodulatory compound, an anti-cancer agent, an anti-viral agent, an anti-bacterial agent, an anti-fungal agent, an anthelmintic agent, or a combination thereof.

In some embodiments, the targeting moiety comprises an —S— moiety. In some embodiments, the targeting moiety is coupled to the remainder of the compound of formula (I) through the —S— moiety.

In certain embodiments, the targeting moiety comprises an antibody, such as an antibody selected from an intact polyclonal antibody, an intact monoclonal antibody, an antibody fragment, a single chain Fv (scFv) mutant, a multispecific antibody, a bispecific antibody, a chimeric antibody, a humanized antibody, a human antibody, a fusion protein comprising an antigenic determinant portion of an antibody, and other modified immunoglobulin molecules comprising antigen recognition sites. For example, the targeting moiety may comprise an antibody selected from Muromonab-CD3, Abciximab, Rituximab, Daclizumab, Palivizumab, Infliximab, Trastuzumab (herceptin), Etanercept, Basiliximab, Gemtuzumab ozogamicin, Alemtuzumab, Ibritumomab tiuxetan, Adalimumab, Alefacept, Omalizumab, Efalizumab, Tositumomob-I¹³¹, Cetuximab, Bevacizumab, Natalizumab, Ranibizumab, Panitumumab, Eculizumab, Rilonacept, Certolizumab pegol, Romiplostim, AMG-531, CNTO-148, CNTO-1275, ABT-874, LEA-29Y, Belimumab, TACI-Ig, Second generation anti-CD20, ACZ-885, Tocilizumab, Atlizumab, Mepolizumab, Pertuzumab, Humax CD20, Tremelimumab (CP-675 206), Ticilimumab, MDX-010, IDEC-114, Inotuzumab ozogamycin, HuMax EGFR, Aflibercept, HuMax-CD4, Ala-Ala, ChAglyCD3, TRX4, Catumaxomab, IGN101, MT-201, Pregovomab, CH-14.18, WX-G250, AMG-162, AAB-001, Motavizumab, MEDI-524, Efumgumab, Aurograb, Raxibacumab, Third generation anti-CD20, LY2469298, and Veltuzumab.

Methods of the Disclosure

The present invention relates to a method of conjugation of proteins to active moieties, employing activated Michael acceptor (AMA) compounds. AMAs, such as vinyl aryl ketones of formulas (II) and (III), can undergo Michael addition reaction with a Michael donor to give conjugates of formulas (IIa) and (IIIa) (Scheme 1).

In the compounds of formulas (II) and (III) L is a spacer moiety; Q is an active moiety or a reactive group; M is N, CR³⁰, or C(-L-Q); J is a targeting moiety; R³⁰ and R³¹ are each independently selected from an electron-withdrawing group, hydrogen, alkyl, alkenyl, alkynyl, aryl, heteroaryl, carbocyclyl, heterocyclyl, and haloalkyl; and R³² is selected from hydrogen, alkyl, alkenyl, alkynyl, aryl, heteroaryl, carbocyclyl, heterocyclyl, and haloalkyl.

In some embodiments of the present invention the Michael donor is a Cys residue of a protein, such as an antibody, or a fusion protein.

In certain embodiments, the Michael donor covalently bound to the targeting moiety is selected from: —SH, —NH₂, —OH,

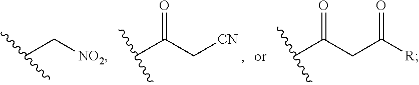

wherein R is C₁₋₃ alkyl or C₁₋₃ alkoxy.

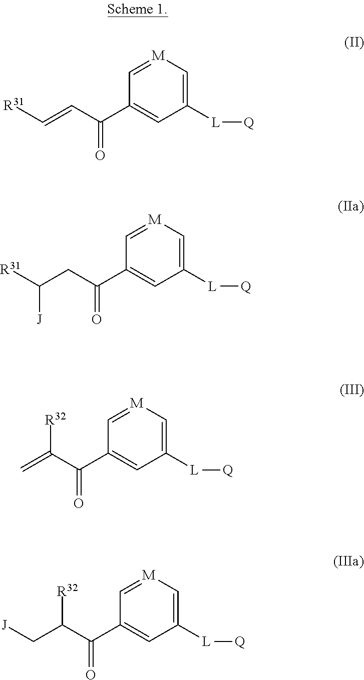

Surprisingly, the inventors discovered that introducing a nitrogen atom or an electron-withdrawing substituent at the meta position of the aryl ring of compound (II) or (III) (the atom denoted “M”) and/or introducing an electron-withdrawing substituent on the Michael acceptor itself (the groups denoted “R³¹”) greatly increases the rate of the Michael addition reaction, resulting in a rapid and clean formation of the conjugate compound (IIa) and (IIIa). An example of the AMA compound 6 cleanly reacting with N-Ac-Cys is shown in Scheme 2.

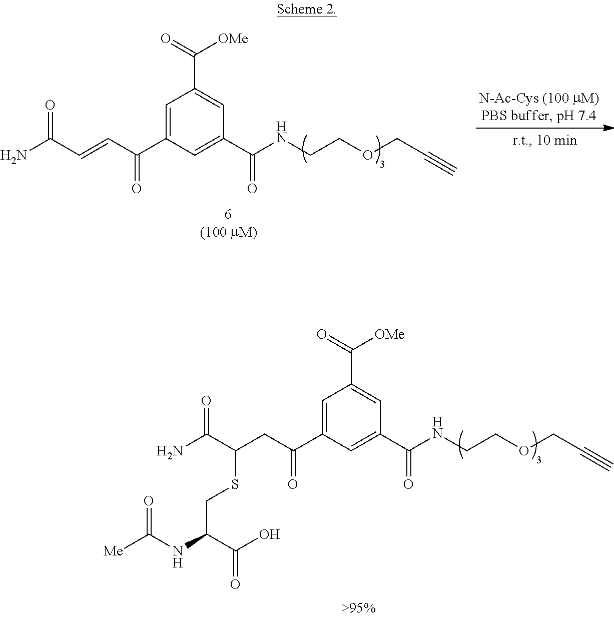

The reactions in Scheme 3 demonstrate chemoselectivity of the reaction of AMAs with N-Ac-Cys, compared to the reaction with N-Ac-Lys and N-Ac-Tyr.

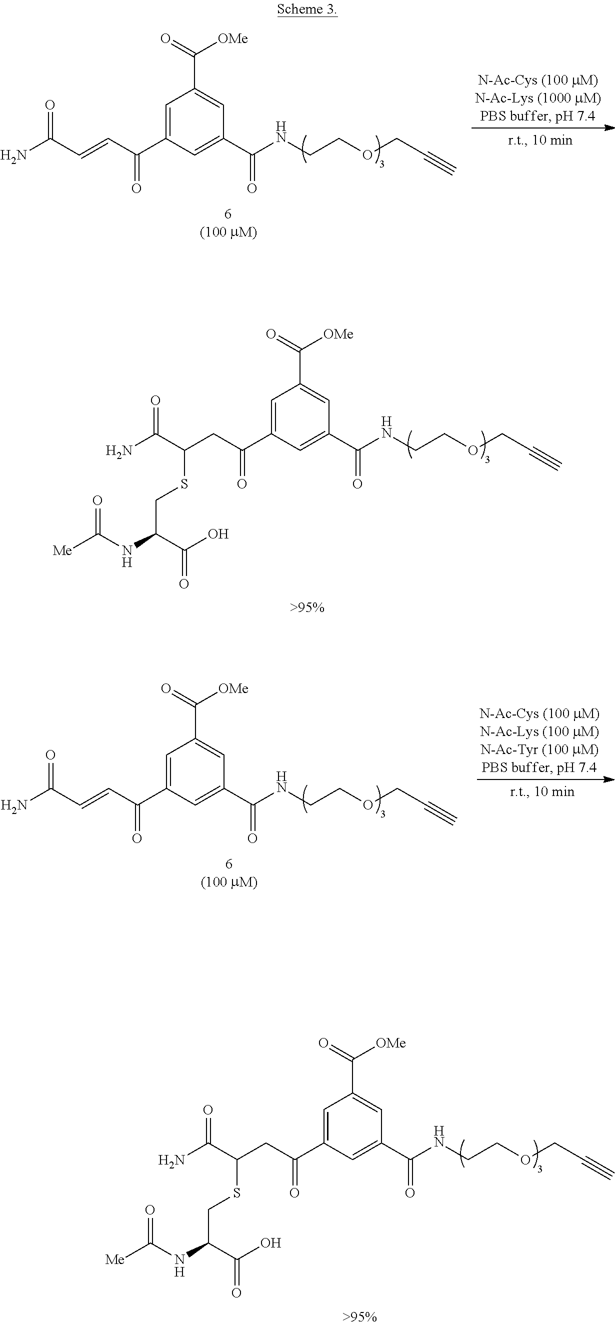

When exposed to a mixture containing equimolar amounts of N-Ac-Cys and N-Ac-Lys (Scheme 3, top reaction) or and N-Ac-Cys, N-Ac-Tyr, and N-Ac-Lys (Scheme 3, bottom reaction), AMA compound 6 produced a conjugate with N-Ac-Cys in over 95% chemoselectivity.

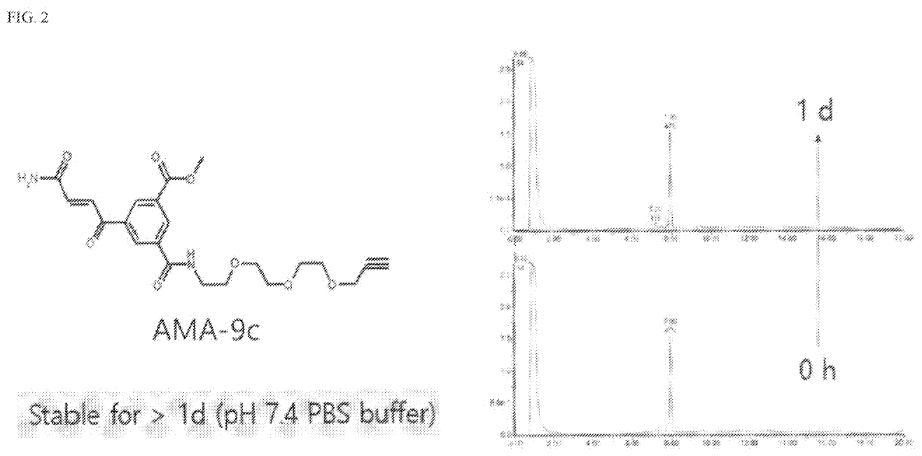

FIG. 2 demonstrates stability of the compounds of formula (II) in aqueous environment. Additional Michael acceptor compounds suitable the for rapid and clean formation of protein conjugates are shown in Scheme 4.

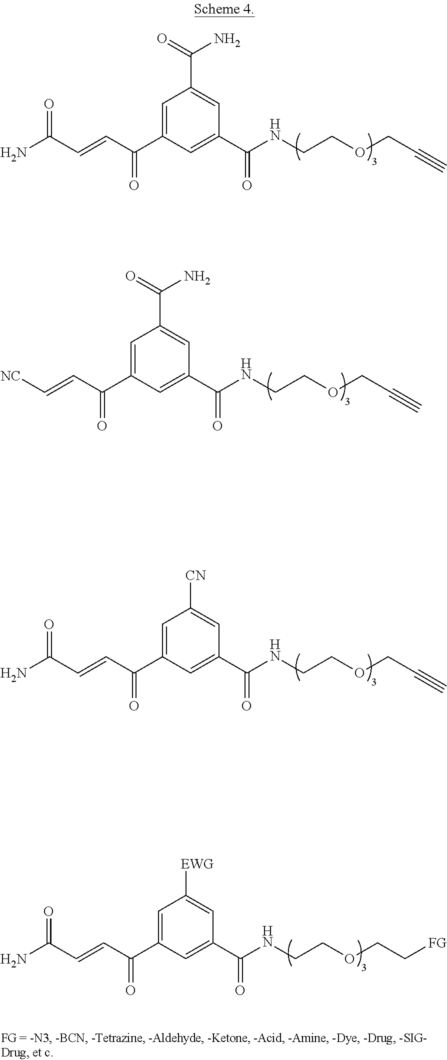

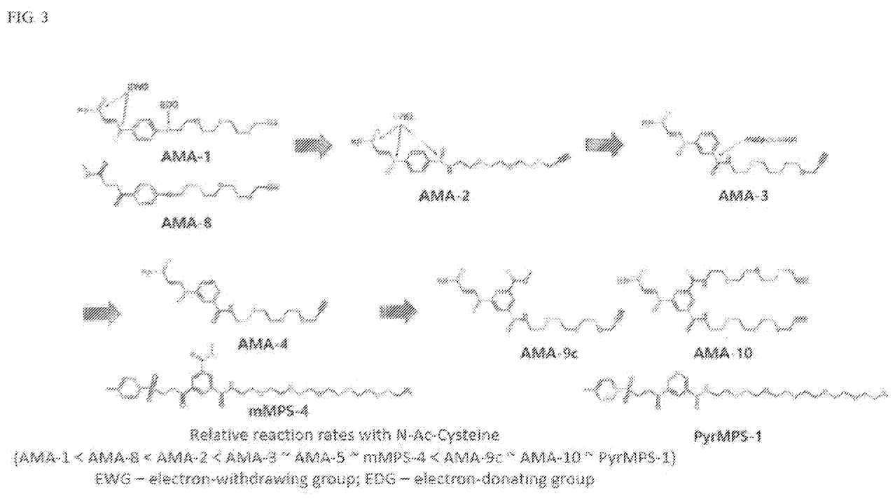

Relative rates of the Michael addition reaction for various compounds of formula (II) as well as other Michael acceptors with N-Ac-Cys are shown in FIG. 3. As shown, placing the Michael acceptor moiety in meta position with respect to the linker substituent on the aryl ring increases the reaction rate (see compound 3 vs. compound 4). The reaction rate increases further in the presence of an electron-withdrawing substituent or a nitrogen atom in a meta position with respect to both the Michael acceptor moiety and the linker moiety on the aryl ring (compounds 5 and 6 vs. compound 4).

In order to address the issue of ADC instability with respect to retro-Michael transformation, various approaches to stabilization of the compounds of formula (IIIa) in aqueous environments were explored. Treating the compounds of formula (IIIa) with a hydride source (such as NaBH₄) results in the formation of an alcohol-containing compound of formula (IVa) (Scheme 5), which is stable in aqueous media. Additionally, the inventors found that treating the compounds of formula (IIIa) with an amine-containing compound or hydroxylamine results in the formation of an imine or an oxime of formula (IVb) (Scheme 5), which is stable in aqueous media (Scheme 6).

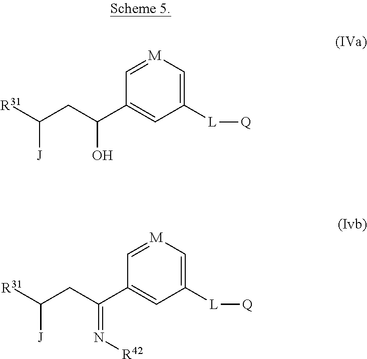

R⁴² is selected from —OH, alkoxy, —NR⁴⁴R⁴⁵, alkyl, alkenyl, alkynyl, aryl, heteroaryl, carbocyclyl, and heterocyclyl, wherein R⁴⁴ and R⁴⁵ together with the nitrogen atom to which they are attached can form a 5-8-membered cycle, optionally fused with an aryl or a heteroaryl ring.

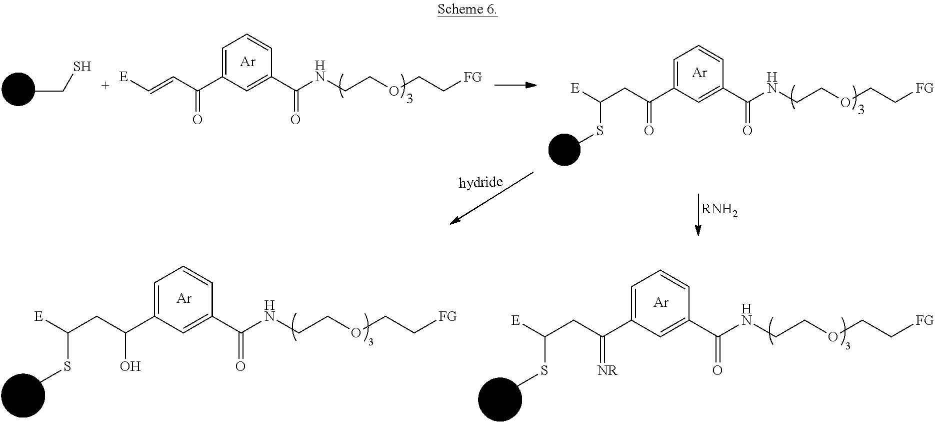

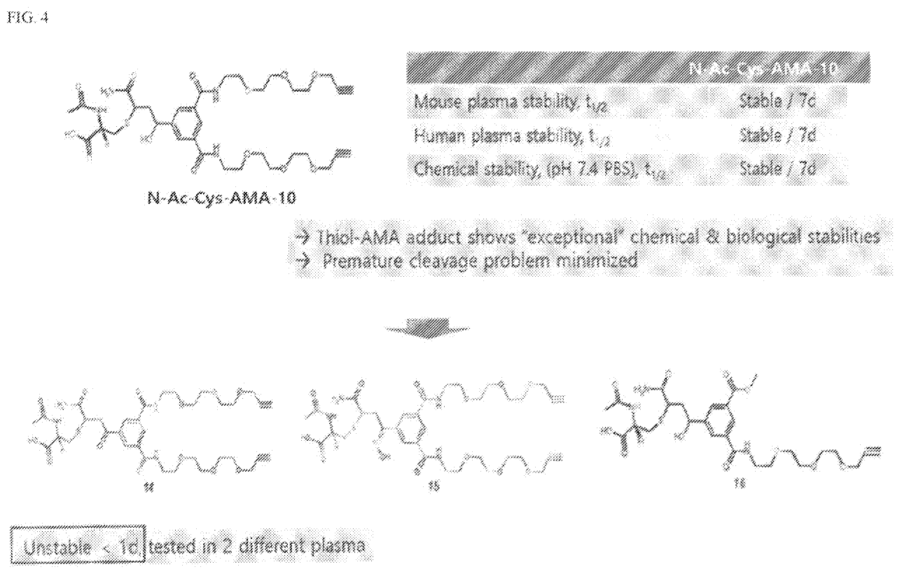

Compounds of formula (IVa) exhibit high stability in human and mouse plasma, as well as in PBS pH 7.4 buffer for over a week, while the corresponding compounds of formula (IIIa) are less stable (FIG. 4).

In certain embodiments, the present disclosure further relates to a method of conjugation of proteins to active moieties, employing a precursor to activated Michael acceptor (AMA) compounds, such as compounds of formulas (Va), (Vb), and (Vc) in Scheme 7, where

X is selected from —Cl, —Br, and —I;

R⁴⁶ is selected from alkyl, alkenyl, alkynyl, aryl, heteroaryl, carbocyclyl, heterocyclyl, and haloalkyl;

R⁴⁷ is O⁻, alkyl, alkenyl, alkynyl, aryl, heteroaryl, carbocyclyl, heterocyclyl; and

n is 1 to 4.

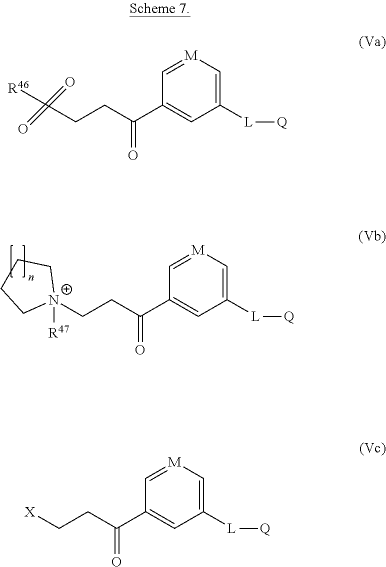

Examples of compounds of formulas (Va), (Vb), and (Vc) are shown in Scheme 8. Compounds of formulas (Va), (Vb), and (Vc) can be cleanly converted into Michael acceptor reagents suitable for undergoing conjugation with a Michael donor-containing molecule, such as a protein.

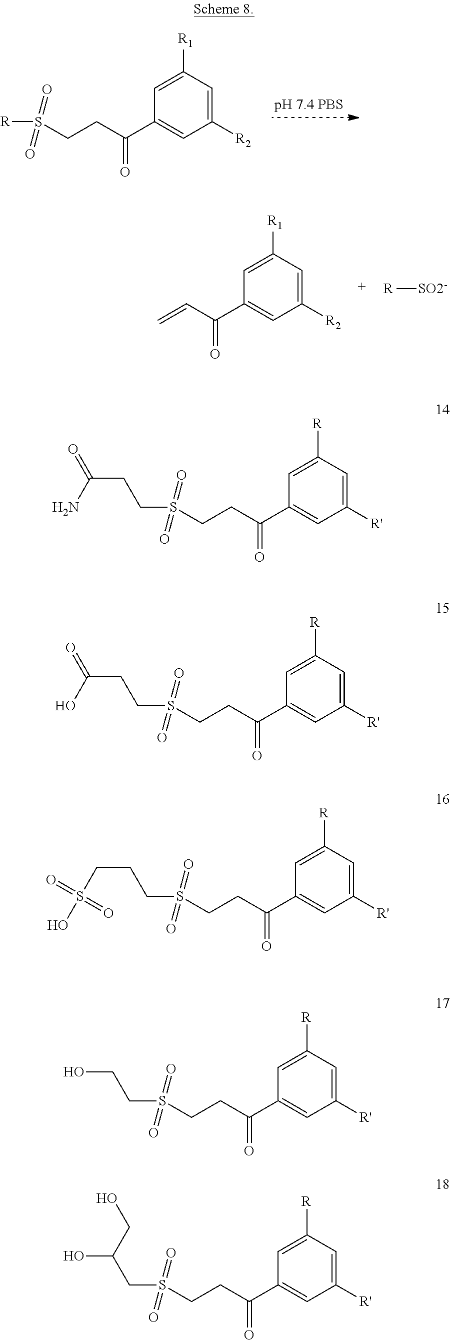

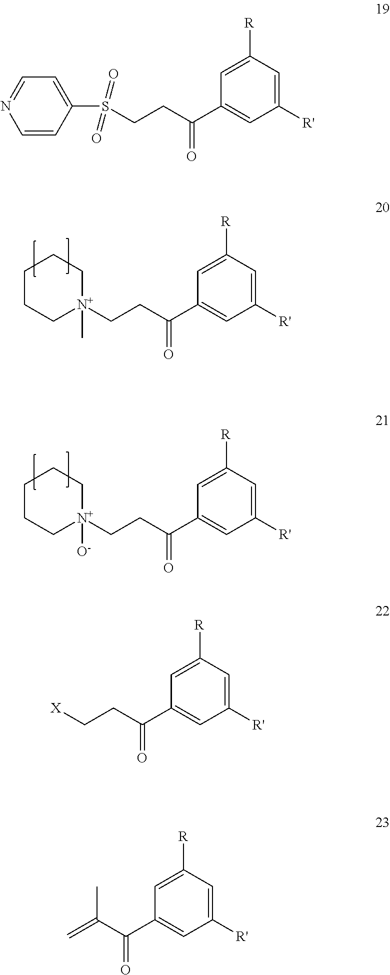

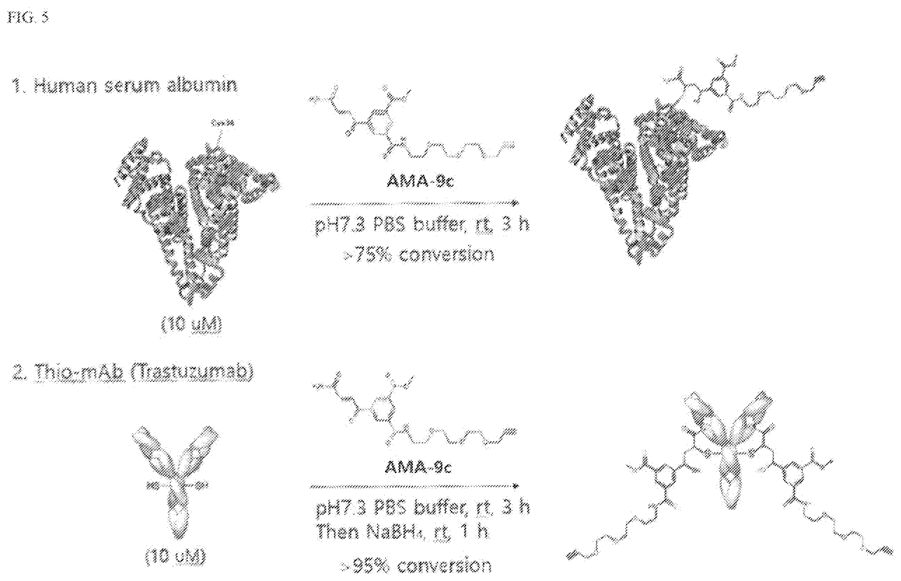

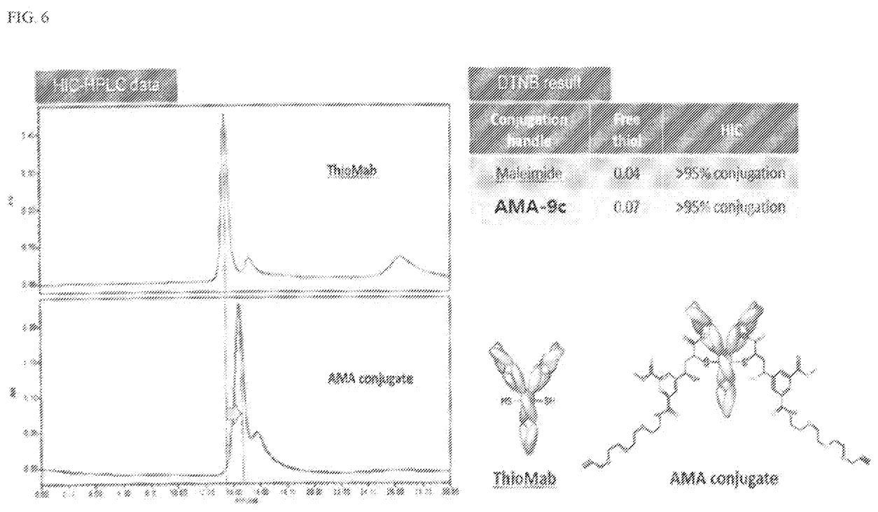

Further studies demonstrate, that the conjugation approach involving AMAs can be extended to antibodies, such as Human Serum Albumin and Thio-mAb (Trastuzumab) (FIGS. 5 and 6). Reduction of the resulting conjugates with NaBH₄ provides plasma-stable ADCs.

In certain embodiments, the present disclosure relates to a compound of formula (I):

or a salt thereof, wherein:

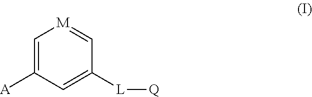

### A is

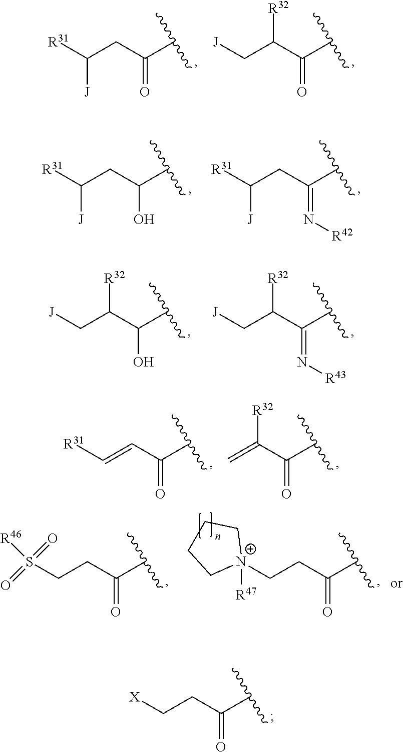

### M is N, CR³⁰, or C(-L-Q);

each L is independently selected from a spacer moiety;
each Q is independently selected from an active moiety or a reactive group;
X is selected from —Cl, —Br, and —I;
J is a targeting moiety;
R³⁰ and R³¹ are each independently selected from an electron-withdrawing group, hydrogen, alkyl, alkenyl, alkynyl, aryl, heteroaryl, carbocyclyl, heterocyclyl, and haloalkyl;
R⁴⁶ is selected from selected from alkyl, alkenyl, alkynyl, aryl, heteroaryl, carbocyclyl, heterocyclyl, and haloalkyl;
R⁴² and R⁴³ are each independently selected from —OH, alkoxy, —NR⁴⁴R⁴⁵, alkyl, alkenyl, alkynyl, aryl, heteroaryl, carbocyclyl, and heterocyclyl, wherein R⁴⁴ and R⁴⁵ together with the nitrogen atom to which they are attached can form a 5-8-membered cycle, optionally fused with an aryl or a heteroaryl ring;
R³², R⁴⁴, and R⁴⁵ are each independently selected from hydrogen, alkyl, alkenyl, alkynyl, aryl, heteroaryl, carbocyclyl, heterocyclyl, and haloalkyl;
R⁴⁷ is O⁻, alkyl, alkenyl, alkynyl, aryl, heteroaryl, carbocyclyl, heterocyclyl; and n is 1 to 4.

In some embodiments, M is N.

In certain embodiments, M is CR³⁰, and R³⁰ is an electron-withdrawing group.

In some embodiments, A is selected from

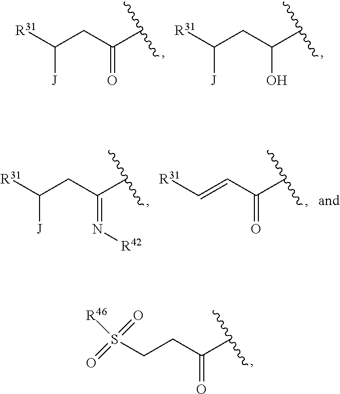

wherein R³¹ is an electron-withdrawing group, preferably wherein L is coupled to C by an electron-withdrawing group selected from an amide or an ester.

In some embodiments, M is C(-L-Q), and wherein L is coupled to C by an electron-withdrawing group.

In some embodiments, R³⁰ is —CO₂NR³³R³⁴ or —CO₂R³⁵, and R³³, R³⁴, and R³⁵ are each independently selected from hydrogen, alkyl, alkenyl, alkynyl, aryl, heteroaryl, carbocyclyl, heterocyclyl, and haloalkyl.

In some embodiments, each electron-withdrawing group is independently selected from —NO₂, —CN, -haloalkyl, —CO₂NR³³R³⁴, —CO₂R³⁵, —C(═O)R³⁶, —S(═O)R³⁷, —S(═O)₂OR³⁸, and —NR³⁹R and R³⁶, R³⁷, R³⁸, R³⁹, R⁴⁰, and R⁴¹ are each independently selected from hydrogen, alkyl, alkenyl, alkynyl, aryl, heteroaryl, carbocyclyl, heterocyclyl, and haloalkyl.

In certain embodiments, each electron-withdrawing group is independently selected from —CN, —CONR³³R³⁴, and —CO₂R³⁵.

In some embodiments, each electron withdrawing group is independently selected from —CN, —CONH₂, and —CO₂Me.

In certain embodiments, Q is an active moiety.

In some embodiments, Q comprises L′ and Q′, wherein L′ is a linker and Q′ is an active agent.

In certain embodiments, L′ comprises a coupling group, wherein the coupling group is coupled to L.

In some embodiments, the coupling group is selected from —C(═O)NR³²—, —C(═O)O—, —C(═NR³²)—, —C═NO—, —NR³²—C(═O)—NR³²—, —OC(═O)O—, —S—S—, —NR³²S(═O)₂O—, and —OS(═O)₂O—.

In certain embodiments, the coupling group is selected from

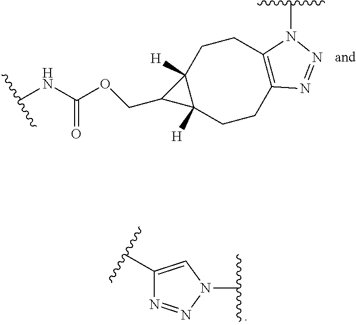 [SMILES: *NC(=O)OCC1[C@H]2CCc3nnn(*)c3CC[C@@H]12 ; *c1cn(*)nn1]

In some embodiments, L′ further comprises a cleavable group, wherein the cleavable group is coupled to Q′.

In certain embodiments, the cleavable group coupled to Q′ is selected from

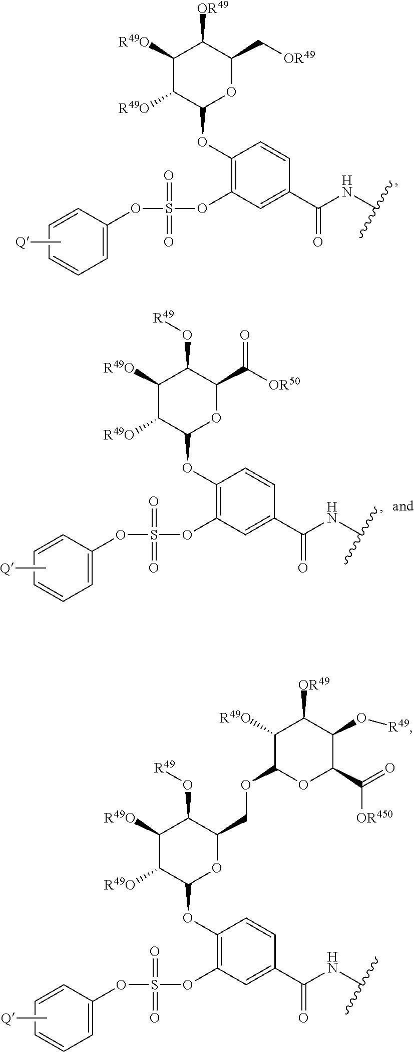

wherein
R⁴⁹ is hydrogen or —C(═O)R⁵⁰; and
R⁵⁰ is lower alkyl.

In some embodiments, L′ further comprises a C₆-C₁₀₀ alkylene comprising at least one group selected from —NH—, —C(═O)—, —O—, —S—, —S(O)—, and —S(═O)₂—.

In certain embodiments, L comprises a C₆-C₁₀₀ alkylene comprising at least one group selected from —NH—, —C(═O)—, —O—, —S—, —S(O)—, and —S(═O)₂—. For example, L comprises

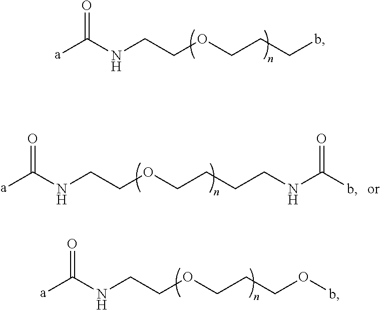

wherein
a is the bond to the M-containing aromatic ring, and b is the bond to L′; and n is 2-20.

In some embodiments, Q′ is a hormone, an oligonucleotide, a toxin, an affinity ligand, a probe for detection, or a combination thereof.

In certain embodiments, Q′ is selected from a cytokine, an immunomodulatory compound, an anti-cancer agent, an anti-viral agent, an anti-bacterial agent, an anti-fungal agent, an anthelmintic agent, or a combination thereof.

In certain embodiments, Q is a reactive group.

I In some embodiments, the reactive group is selected from —N₃, —C═CH

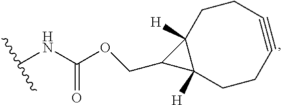 [SMILES: *NC(=O)OCC1[C@H]2CCC#CCC[C@@H]12]

—S(O)₂Hal, —NH₂, —CO₂Hal, —OH, —C(O)H, —SH, —N═C═O, and —N═S═C, wherein Hal is —Cl, —Br, or —I.

In certain embodiments, the targeting moiety comprises an —S— moiety.

In some embodiments, the targeting moiety is coupled to the remainder of the compound of formula (I) through the —S— moiety.

In some embodiments, A is

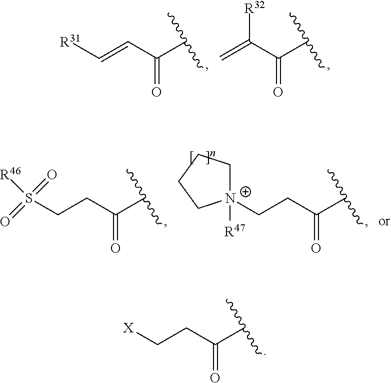

In some embodiments, A is

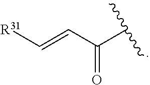

In certain embodiments, R³¹ is —CN, —CO₂NR³³R³⁴, or —CO₂R³⁵.

In certain embodiments, A is

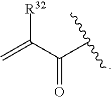

In some embodiments, R³² is hydrogen or C₁₋₃ alkyl.

In some embodiments, A is

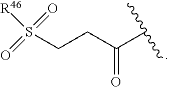

In certain embodiments, R⁴⁶ is optionally substituted C₁₋₃ alkyl, optionally substituted C₆-C₁₂ aryl, or optionally substituted heteroaryl.

In some embodiments, A is

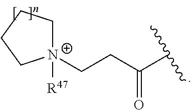

In certain embodiments, R⁴⁷ is O⁻or C₁₋₃ alkyl.

In certain embodiments, A is

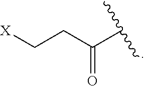

In some embodiments, A is

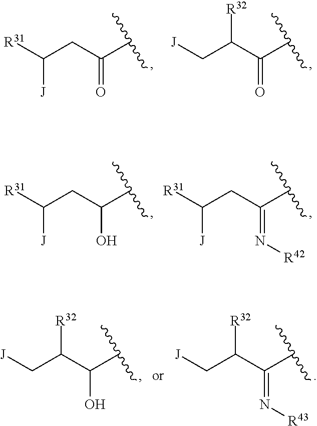

For example, A may be

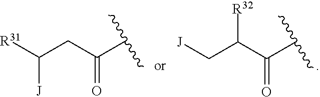

Alternatively, A may be

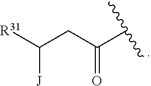

In other embodiments, A may be

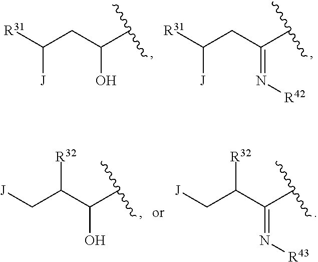

In some embodiments, A is

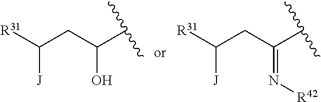

In certain embodiments, R⁴² is —OH or —NR⁴⁴R⁴⁵.

In some embodiments, the targeting moiety comprises a nanoparticle, an immunoglobulin, a nucleic acid, a protein, an oligopeptide, a polypeptide, an antibody, a fragment of an antigenic polypeptide, or a repebody. For example, the targeting moiety may comprise an antibody, such as an antibody selected from an intact polyclonal antibody, an intact monoclonal antibody, an antibody fragment, a single chain Fv (scFv) mutant, a multispecific antibody, a bispecific antibody, a chimeric antibody, a humanized antibody, a human antibody, a fusion protein comprising an antigenic determinant portion of an antibody, and modified immunoglobulin molecules comprising antigen recognition sites. For example, the targeting moiety may comprise an antibody selected from Muromonab-CD3, Abciximab, Rituximab, Daclizumab, Palivizumab, Infliximab, Trastuzumab (herceptin), Etanercept, Basiliximab, Gemtuzumab ozogamicin, Alemtuzumab, Ibritumomab tiuxetan, Adalimumab, Alefacept, Omalizumab, Efalizumab, Tositumomob-I¹³¹, Cetuximab, Bevacizumab, Natalizumab, Ranibizumab, Panitumumab, Eculizumab, Rilonacept, Certolizumab pegol, Romiplostim, AMG-531, CNTO-148, CNTO-1275, ABT-874, LEA-29Y, Belimumab, TACI-Ig, Second generation anti-CD20, ACZ-885, Tocilizumab, Atlizumab, Mepolizumab, Pertuzumab, Humax CD20, Tremelimumab (CP-675 206), Ticilimumab, MDX-010, IDEC-114, Inotuzumab ozogamycin, HuMax EGFR, Aflibercept, HuMax-CD4, Ala-Ala, ChAglyCD3, TRX4, Catumaxomab, IGN101, MT-201, Pregovomab, CH-14.18, WX-G250, AMG-162, AAB-001, Motavizumab, MEDI-524, Efumgumab, Aurograb, Raxibacumab, Third generation anti-CD20, LY2469298, and Veltuzumab.

In some embodiments, the compound of formula (I) is selected from

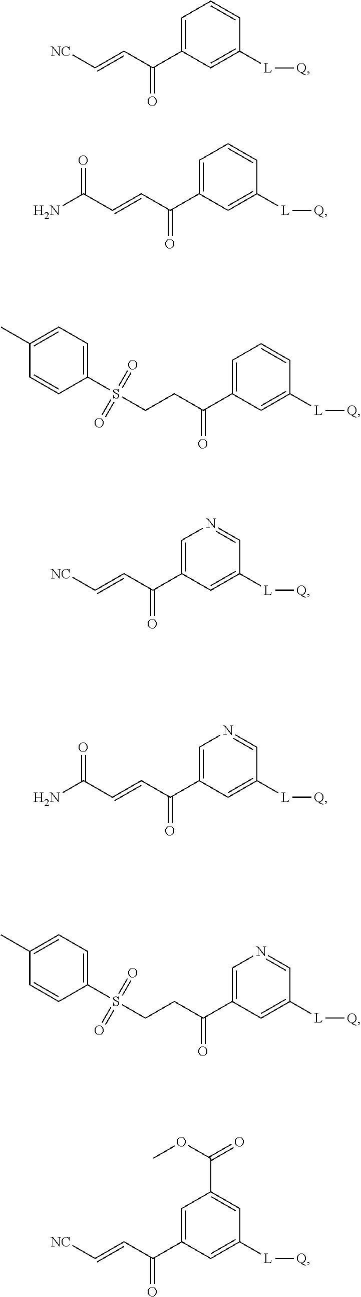

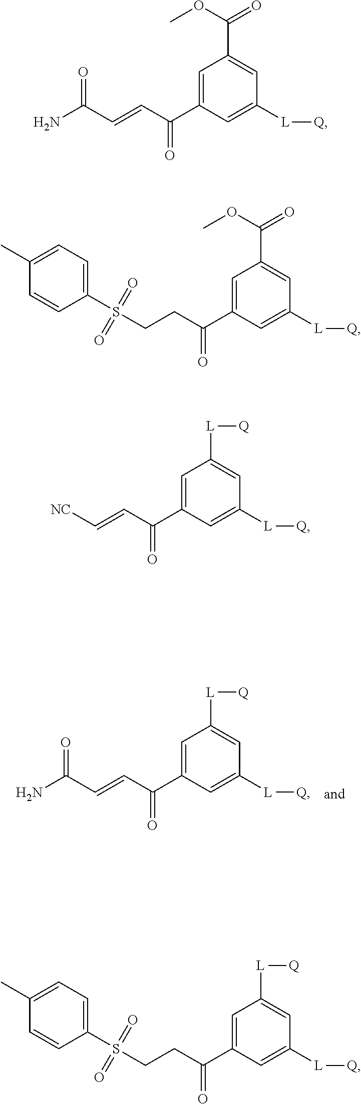

In some embodiments, the present disclosure relates to a method of preparing a conjugate, comprising reacting the compound of formula (I) with a reagent comprising a targeting moiety covalently bound to a Michael donor, thereby producing a Michael adduct.

In some embodiments, the present disclosure relates to a method further comprising reducing the Michael adduct.

In some embodiments, the Michael donor covalently bound to the targeting moiety is selected from: —SH, —NH₂, —OH,

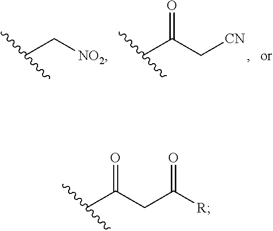

wherein R is C₁₋₃ alkyl or C₁₋₃ alkoxy.

In some embodiments, the present disclosure relates to a pharmaceutical composition comprising a compound of formula (I) or a pharmaceutically acceptable salt thereof, and a pharmaceutically acceptable excipient.

In some embodiments, the present disclosure relates to a method for treating a disease or disorder, comprising administering a compound of formula (I) or a pharmaceutically acceptable salt thereof, or a composition comprising a compound of formula (I) or a pharmaceutically acceptable salt thereof, and a pharmaceutically acceptable excipient to a subject in need thereof.

In some embodiments, the disease or disorder is selected from cancer, infectious disease, or autoimmune disease.

In certain embodiments, the disease or disorder is cancer.

### Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing one or more hydrogens on one or more carbons of the hydrocarbon backbone.

Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a guanidino, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “Cₓ-C_{y}” when used in conjunction with a chemical moiety, such as, acyl, acyloxy, alkyl, alkenyl, alkynyl, or alkoxy is meant to include groups that contain from x to y carbons in the chain. For example, the term “Cₓ-C_{y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C⁰ alkyl indicates a hydrogen where the group is in a terminal position, a bond if internal. The terms “C₂-C_{y}alkenyl” and “C₂-C_{y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

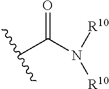

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

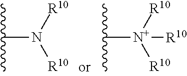

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Accordingly, the term “aryl” can encompass (C₅-C₁₀) and (C₆-C₁₀) aryl groups. Aryl groups include benzene, naphthalene, phenanthrene, phenol, aniline, and the like.

The term “carbamate” is art-recognized and refers to a group

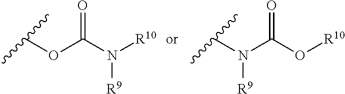

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be susbstituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “(cycloalkyl)alkyl”, as used herein, refers to an alkyl group substituted with a cycloalkyl group.

The term “carbonate” is art-recognized and refers to a group —OCO₂-R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The term “heteroaralkyl”, as used herein, refers to an alkyl group substituted with a heteroaryl group.

The term “heteroalkyl”, as used herein, refers to a saturated or unsaturated chain of carbon atoms and at least one heteroatom, wherein no two heteroatoms are adjacent.

The term “heteroaryl” includes substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Accordingly, the term “heteroaryl” can encompass (C₂-C₁₀) and (C₂-C₁₀) heteroaryl groups. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocycloalkyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocycloalkyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocycloalkyl groups include, for example, piperidine, piperazine, pyrrolidine, morpholine, lactones, lactams, and the like.

The term “(heterocycloalkyl)alkyl”, as used herein, refers to an alkyl group substituted with a heterocycloalkyl group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

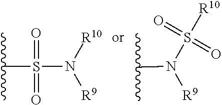

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂-R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

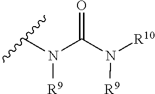

wherein R⁹ and R₁₀ independently represent hydrogen or a hydrocarbyl, such as alkyl, or either occurrence of R⁹ taken together with R¹⁰ and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts,

Protective Groups in Organic Chemistry, 3rd Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). “Conjugate” refers to two or more molecules that are covalently linked into a larger construct. In some embodiments, a conjugate includes one or more biomolecules (such as peptides, nucleic acids, proteins, enzymes, sugars, polysaccharides, lipids, glycoproteins, and lipoproteins) covalently linked to one or more other molecules, such as one or more other biomolecules or polymer linkers. “Conjugating,” “joining,” “bonding,” “coupling” or “linking” are used synonymously to mean joining a first atom or molecule to another atom or molecule to make a larger molecule either directly or indirectly.

As used herein, the term “antibody-drug conjugate (ADC)” refers to the molecule in which a drug and an antibody are chemically bound to each other, e.g. through a linker moiety, without reducing the biological activities of the antibody and the drug.

As used herein, the term “antibody” refers to a protein molecule which comprises an immunoglobulin, immunoglobulin chimera, or immunoglobulin-like molecule (including by way of example and without limitation, IgA, IgD, IgE, IgG and

IgM, combinations thereof, and similar molecules produced during an immune response in any vertebrate, (e.g., in mammals such as humans, goats, rabbits and mice) and antibody fragments that specifically bind to a molecule of interest (or a group of highly similar molecules of interest) to the substantial exclusion of binding to other molecules. The term is intended to encompass polyclonal antibodies, monoclonal antibodies, full-length antibodies and antibody fragments containing antigen binding domains. A full-length antibody has two full-length light chains and two full-length heavy chains, in which each of the light chains is linked to the heavy chain by a disulfide bond. The full-length antibody comprises IgA, IgD, IgE, IgM and IgG, and subtypes of IgG include IgG1, IgG2, IgG3 and IgG4. The term “antibody fragment” refers to a fragment having an antigen-binding function, and is intended to include recombinant antibody fragments (such as sFv fragments, dsFv fragments, bispecific sFv fragments, bispecific dsFv fragments, F(ab)′2 fragments, single chain Fv proteins (“scFv”), disulfide stabilized Fv proteins (“dsFv”), diabodies, and triabodies (as are known in the art), and camelid antibodies (see, for example, U.S. Pat. Nos. 6,015,695; 6,005,079, 5,874,541; 5,840,526; 5,800,988; and 5,759,808). Fab comprises light-chain and heavy-chain variable regions, a light-chain constant region, and a heavy-chain first constant domain (CH1), and has one antigen-binding site. Fab′ has a hinge region including one or more cysteine residues at the C-terminus of the heavy-chain CH1 domain. A F(ab′)₂ antibody is contains a disulfide bond between the cysteine residues of the hinge region of Fab′. Fv refers to a minimal antibody fragment having only a heavy-chain variable region and a light-chain variable region. dsFv is has a structure in which a heavy-chain variable region and a light-chain variable region are linked to each other by a disulfide bond, and scFv generally has a structure in which a heavy-chain variable region and a light-chain variable region are covalently linked to each other by a peptide linker. These antibody fragments can be obtained using proteases (for example, Fab fragments can be obtained by digesting a full-length antibody with papain, and F(ab′)₂ fragments can be obtained by digesting a full-length antibody with pepsin). Preferably, these antibody fragments can be produced by a genetic recombinant technique.

The term “antibody” includes monoclonal antibody which are produced by a single clone of B lymphocytes or by a cell into which the light and heavy chain genes of a single antibody have been transfected. Monoclonal antibodies may be obtained using a variety of techniques known to those skilled in the art, including standard hybridoma technology (see, e.g., Kohler and Milstein, Eur. J. Immunol., 5: 511-519 (1976), Harlow and Lane (eds.), Antibodies: A Laboratory Manual, CSH Press (1988), and C. A. Janeway et al. (eds.), Immunobiology, 5th Ed., Garland Publishing, New York, N.Y. (2001)). Monoclonal antibodies also may be generated using other suitable techniques including EBV-hybridoma technology (see, e.g., Haskard and Archer, J. Immunol. Methods, 74(2): 361-67 (1984), and Roder et al., Methods Enzymol., 121: 140-67 (1986)), bacteriophage vector expression systems (see, e.g., Huse et al., Science, 246: 1275-81 (19891), or phage display libraries comprising antibody fragments, such as Fab and scFv (single chain variable region) (see, e.g., U.S. Pat. Nos. 5,885,793 and 5,969,108, and International Patent Application Publications WO 92/01047 and WO 99/06587, each of which is incorporated herein by reference in their entirety). Monoclonal antibodies include humanized monoclonal antibodies. As used herein, a “humanized” antibody is one in which the complementarity-determining regions (CDR) of a mouse monoclonal antibody, which form the antigen binding loops of the antibody, are grafted onto the framework of a human antibody molecule. Owing to the similarity of the frameworks of mouse and human antibodies, it is generally accepted in the art that this approach produces a monoclonal antibody that is antigenically identical to a human antibody but binds the same antigen as the mouse monoclonal antibody from which the CDR sequences were derived. Methods for generating humanized antibodies are well known in the art and are described in detail in, for example, Janeway at al., supra, U.S. Pat. Nos. 5,225,539, 5,585,089 and 5,693,761, each of which is incorporated herein by reference in its entirety, European Patent No. 0239400 B1, and United Kingdom Patent No. 2188638. Humanized antibodies can also be generated using the antibody resurfacing technology described in U.S. Pat. No. 5,639,641, which is incorporated herein by reference in its entirety, and Pedersen at al., J. Mol. Biol., 235: 959-973 (1994).

Antibodies that are disclosed in the present invention may be natural antibodies or recombinant antibodies. As used herein, the term “natural antibody” refers to an antibody that has undergone no genetic modification. As used herein, the term “recombinant antibody” refers to a genetically modified antibody which may have an antigen-binding activity or desired characteristic imparted by genetic modification.

As used herein, the term “cytokine” refers to small cell-signaling protein molecules that are secreted by numerous cells and are a category of signaling molecules used extensively in intercellular communication. Cytokines can be classified as proteins, peptides, or glycoproteins; the term “cytokine” encompasses a large and diverse family of regulators produced throughout the body by cells of diverse embryological origin.

As used herein, the term “hormone” relates to a chemical released by a cell, a gland, or an organ in one part of the body, that perform a signaling function for the cells in other parts of the organism. The term encompasses peptide hormones, lipid and phospholipid-derived hormones including steroid hormones, and monoamines.

“Moiety” refers to a fragment of a molecule, or a portion of a molecule, e.g., a conjugate.

The present invention discloses compounds with functional groups capable of undergoing a Michael addition reaction. Michael addition is taught, for example, by R.

T. Morrison and R. N. Boyd in Organic Chemistry, third edition, Allyn and Bacon, 1973. The reaction takes place between a molecule comprising a Michael donor moiety and a molecule comprising a Michael acceptor moiety.

Target-Oriented Treatments The targeting moiety of the conjugate may recognize a cell, or be recognized by a cell, thereby providing a so-called target-oriented treatment.

In some embodiments, the conjugate comprises an active moiety Q for use in a target-oriented treatment for treating an autoimmune disease. In certain embodiments, the active moiety comprises an active agent selected from: cyclosporine, cyclosporine A, mycophenylate mofetil, sirolimus, tacrolimus, enanercept, prednisone, azathioprine, methotrexate cyclophosphamide, aminocaproic acid, chloroquine, hydroxychloroquine, hydrocortisone, dexamethasone, chlororambucil, DHEA, danazol, bromocriptine, meloxicam, or infliximab.

In some embodiments, the compound comprises an active moiety Q for use in a target-oriented treatment for treating an infectious disease. In certain embodiments, Q comprises an active agent selected from: a beta-lactam (e.g., penicillin G, penicillin V, cloxacillin, dicloxacillin, methicillin, nafcillin, oxacillin, ampicillin, amoxicillin, becampicillin, azlocillin, carbenicillin, mezlocillin, piperacillin, ticarcillin), aminoglycoside series (e.g., amikacin, gentamycin, kanamycin, neomycin, netilmicin, streptomycin, tobramycin), macrolide series (azithromycin, clarithromycin, erythromycin, lincomycin, clindamycin), a tetracycline (e.g., demeclocycline, doxycyline, minocycline, tetracycline), a quinolone (e.g., cinoxacin, nalidixic acid), a fluoroquinolone (e.g., ciprofloxacin, enoxacin, grepafloxacin, levofloxacin, lomefloxacin, norfloxacin, ofloxacin, sparfloxacin, trovafloxicin), a polypeptide (e.g., bacitracin, colistin,polymyxin B), a sulfonamide (e.g., sulfisoxazole, sulfamethoxazole, sulfadiazine, sulfamethizole, sulfacetamide), an antibiotic agent, such as trimethoprim, sylfamethazole, chloramphenicol, vancomycin, metronidazole, quinupristin, dalfopristin, rifampicin, spectinomycin, or nitrofurantoin, a general anti-viral agent (e.g., idoxuradine, vidarabine, acyclovir, famcicyclovir, pencicyclovir, valacyclovir, gancicyclovir, foscarnet, ribavirin, amantadine, rimantadine, cidofovir, Antisense oligonucleotides, Immunoglobumins, interfeones), an HIV infection therapeutic agent (e.g., tenofovir, emtricitabine, zidovudine, didanosine, zalcitabine, stavudine, lamivudine, nevirapine, delaviridine, saquinavir, ritonavir, indinavir, nelfinavir).

In some embodiments, the compounds and conjugates disclosed herein comprise an active moiety Q for use in a method for delivering an active agent to a cell for treating a tumor, wherein the targeting moiety is selected to bind with a target cell (i.e., a cancer cell). In particular, the present compounds, conjugates, and compositions may be useful for inhibiting abnormal cell growth or treating a proliferative disorder in a mammal (e.g., a human), such as where the target cell is a cancer cell and the targeting moiety is selected to bind to molecule associated with the cancer cell (and not associated with healthy cells, or at least preferentially associated with tumor cells rather than healthy cells).

In certain embodiments, the active moiety Q comprises a cytotoxic or immunomodulatory agent, an anti-cancer agent, an anti-tubulin agent, or a cytotoxic agent Preferably, the cytotoxic or immunomodulatory agent is selected from an anti-tubulin agent, auristatin, a DNA minor groove binder, a DNA transcription inhibitor, an alkylating agent, anthracycline, antibitiotic, antifolate, antimetabolite, a calmodulin inhibitor, a chemotherapy sensitizer, duocarmycin, etoposide, fluorindated pyrimidine, ionophore, lexitropsin, maytansinoid, nitrosourea, platinol, a pore-forming compound, purine antimetabolite, puromycin, radiation sensitizer, rapamycin, steroid, taxane, topoisomerase inhibitor, and vinca alkaloid.; the anti-cancer agent is selected from methotrexate, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, proocarbizine, topotecan, nitrogen mustards, cytoxan, etoposide, 5-fluorouracil, BCNU, irinotecan, camptothecins, bleomycin, doxorubicin, idaribicin, daunorubicin, dactinomycin, plicamycin, mitoxantrone, asparaginase, vinblastine, vincristine, vinorelbine, paclitaxel, docetaxel, etc.; the anti-tublin agent includes taxane (e.g., paclitaxel, docetaxel), T67, vinca alkyloid (e.g., vincristine, vinblastine, vindesine, vinorelbine), a baccatin derivative, a taxane derivative, epothiolone (e.g., epothilone A, epothilone B), nocodazole, colchicine, colcimid, estramustine, crytophycins, cemadotin, maytansinoids, combrestatins, discodermolide, eleutherobin, or an auristatin derivative (e.g., AFP, MIVIAF, MMAE);

the cytotoxic agent is selected from androgen, anthramycin(AMC), asparaginase, 5-azacytidine, azathioprine, bleomycin, busulfan, buthionine sulfoximine, calicheamicin, calicheamicin derivative, camptothecin, carboplatin, carmustine (BSNU), CC-1065, chlorambucin, cisplatin, colchicine, cyclophosphamide, cytarabine, cytidine arabinoside, cytochalasin B, dacarbazine, dactinomycin(actinomycin), daunorubicin, decarbazine, DM1, DM4, docetaxel, doxorubicin, etoposide, estrogen, 5-fluordeoxyuridine, 5-fluorouracil, gemcitabine, gramicidin D, hydroxyurea, idaribicin, ifosfamide, irinotecan, lomustine (CCNU), maytansine, mechlorethamine, melphalan, 6-merceptopurine, methotrexate, mithramycin, mitomycin C, mitoxantrone, nitroimidazole, paclitaxel, palytoxin, plicamycin, procarbizine, rhizoxin, streptozotocin, tenoposide, 6-thioguanine, thioTEPA, topotecan, vinblastine, vincristine, vinorelbine, VP-16, VM-26; DNA minor groove binder (e.g., enediyne, lexitropsin, CBI compound), duocarmycin, taxane (e.g., paclitaxel, docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A, epothilone B, estramustine, cryptophycins, cemadotin, maytansinoids, discodermolide, eleutherobin, or mitoxantrone.

### Cellular Proliferation and Apoptosis

The compounds and conjugates disclosed herein may be used in methods to induce apoptosis in cells.

Dysregulated apoptosis has been implicated in a variety of diseases, including, for example, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma or Crohn's disease), hyperproliferative disorders (e.g., breast cancer, lung cancer), viral infections (e.g., herpes, papilloma, or HIV), and other conditions, such as osteoarthritis and atherosclerosis. The compounds, conjugates, and compositions described herein may be used to treat or ameliorate any of these diseases. Such treatments generally involve administering to a subject suffering from the disease an amount of a compound, conjugate, or composition described herein sufficient to provide therapeutic benefit. The identity of the antibody of the compound, conjugate, or composition administered will depend upon the disease being treated—thus the antibody should bind a cell-surface antigen expressed in the cell type where inhibition would be beneficial. The therapeutic benefit achieved will also depend upon the specific disease being treated. In certain instances, the compounds and compositions disclosed herein may treat or ameliorate the disease itself, or symptoms of the disease, when administered as monotherapy. In other instances, the compounds and compositions disclosed herein may be part of an overall treatment regimen including other agents that, together with the inhibitor or the compounds and compositions disclosed herein, treat or ameliorate the disease being treated, or symptoms of the disease. Agents useful to treat or ameliorate specific diseases that may be administered adjunctive to, or with, the the compounds and compositions disclosed herein will be apparent to those of skill in the art.

Although absolute cure is always desirable in any therapeutic regimen, achieving a cure is not required to provide therapeutic benefit. Therapeutic benefit may include halting or slowing the progression of the disease, regressing the disease without curing, and/or ameliorating or slowing the progression of symptoms of the disease. Prolonged survival as compared to statistical averages and/or improved quality of life may also be considered therapeutic benefit.

One particular class of diseases that involve dysregulated apoptosis and that are significant health burden world-wide are cancers. In certain embodiments, the compounds and compositions disclosed herein may be used to treat cancers. The cancer may be, for example, solid tumors or hematological tumors. Cancers that may be treated with the compounds and compositions disclosed herein include, but are not limited to bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, myeloma, prostate cancer, small cell lung cancer and spleen cancer. The compounds and compositions disclosed herein may be especially beneficial in the treatment of cancers because the antibody can be used to target the tumor cell specifically, thereby potentially avoiding or ameliorating undesirable side-effects and/or toxicities that may be associated with systemic administration of unconjugated inhibitors. In some embodiments, the present disclosure relates to a method of treating a disease involving dysregulated intrinsic apoptosis, comprising administering to a subject having a disease involving dysregulated apoptosis an amount of a compound and composition disclosed herein effective to provide therapeutic benefit, wherein the targeting moiety of the compounds and compositions disclosed herein binds a cell surface receptor on a cell whose intrinsic apoptosis is dysregulated. In some embodiments, a method of treating cancer comprises administering to a subject having cancer a compound and composition disclosed herein, wherein the targeting moiety is capable of binding a cell surface receptor or a tumor associated antigen expressed on the surface of the cancer cells, in an amount effective to provide therapeutic benefit.

In the context of tumorigenic cancers, therapeutic benefit, in addition to including the effects discussed above, may also specifically include halting or slowing progression of tumor growth, regressing tumor growth, eradicating one or more tumors and/or increasing patient survival as compared to statistical averages for the type and stage of the cancer being treated. In some embodiments, the cancer being treated is a tumorigenic cancer.

The compounds and conjugates disclosed herein may be administered as monotherapy to provide therapeutic benefit, or may be administered adjunctive to, or conjoint with, other chemotherapeutic agents and/or radiation therapy. Chemotherapeutic agents to which the compounds and compositions disclosed herein may be utilized as adjunctive therapy may be targeted (for example, ADCs, protein kinase inhibitors, etc.) or non-targeted (for example, non-specific cytotoxic agents such as radionucleotides, alkylating agents and intercalating agents). Non-targeted chemotherapeutic agents with which the compounds and compositions disclosed herein may be adjunctively administered include, but are not limited to, methotrexate, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, procarbizine, topotecan, nitrogen mustards, Cytoxan, etoposide, 5-fluorouracil, BCNU, irinotecan, camptothecins, bleomycin, doxorubicin, idarubicin, daunorubicin, dactinomycin, plicamycin, mitoxantrone, asperaginase, vinblastine, vincristine, vinorelbine, paclitaxel, calicheamicin, and docetaxel.

The compounds and conjugates disclosed herein that may not be effective as monotherapy to treat cancer may be administered adjunctive to, or with, other chemotherapeutic agents or radiation therapy to provide therapeutic benefit. In some embodiments, the present disclosure pertains to a method in which a compound or composition disclosed herein is administered in an amount effective to sensitize the tumor cells to standard chemotherapy and/or radiation therapy. Accordingly, in the context of treating cancers, “therapeutic benefit” includes administering the compounds and compositions disclosed herein adjunctive to, or with, chemotherapeutic agents and/or radiation therapy, either in patients who have not yet begin such therapy or who have but have not yet exhibited signs of resistance, or in patients who have begun to exhibit signs of resistance, as a means of sensitizing the tumors to the chemo and/or radiation therapy.

Pharmaceutical Compositions

In certain embodiments, the invention provides a solid pharmaceutical composition comprising a compound of the invention, such as a compound of formula, or a pharmaceutically acceptable salt thereof; and a pharmaceutically acceptable carrier.

In certain embodiments, the present invention provides a pharmaceutical preparation suitable for use in a human patient, comprising any compound of the invention or a pharmaceutically acceptable salt thereof and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

In some embodiments, the present invention relates to a pharmaceutical kit comprising a compound of the invention, or a pharmaceutically acceptable salt thereof, and optionally directions on how to administer the compound.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, non-aqueous vehicles such as glycols, glycerol, oils such as olive oil, or injectable organic esters. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, suppository, or the like. The composition can also be present in a transdermal delivery system, e.g., a skin patch.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) ethyl alcohol; and (17) other non-toxic compatible substances employed in pharmaceutical formulations.

A pharmaceutical composition (preparation) can be administered to a subject by any of a number of routes of administration including, for example, orally (for example, drenches as in non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein, each of which is incorporated herein by reference in its entirety.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in a non-aqueous liquid, or as pastilles (using an inert base, such as gelatin and glycerin, or sucrose and acacia) and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference in their entirety. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable nonaqueous carriers that may be employed in the pharmaceutical compositions of the invention include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Examples

The invention now being generally described, it will be more readily understood by reference to the following examples which are included merely for purposes of illustration of certain aspects and embodiments of the present invention, and are not intended to limit the invention.

### Example 1. Preparation of Compound L-1

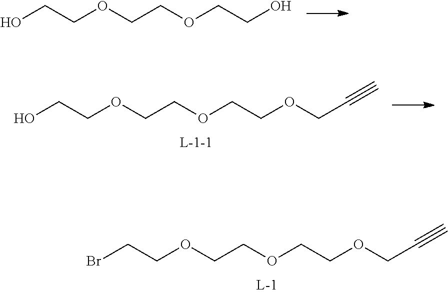

Preparation of Compound L-1-1

A homogeneous solution of triethylene glycol (60.6 g, 403.5 mmol) in dry THF at room temperature under N₂ atmosphere was treated with 60%-NaH (3.2 g, 80.7 mmol) and stirred for 15 minutes. Propargyl bromide (10 g, 67.25 mmol) was added dropwise and the resulting mixture was allowed to stand for overnight. The reaction was quenched with H₂O (250 mL) and extracted with DCM (250 mL×4). The organic layer was washed with brine (500 mL), dried over anhydrous Na₂SO₄, filtered, and concentrated in vacuo. The residue was purified by flash chromatography (EA: HEX=3: 1) to give the title compound L-1-1 (12.5 g, 98%) as a liquid.

¹H NMR (400 MHz, CDCl₃) δ 4.18 (d, J=2.4 Hz, 2H), 3.73-3.64 (m, 10H), 3.60-3.57 (m, 2H), 2.63 (m, 1H), 2.42 (m, 1H).

Preparation of Compound L-1

A homogeneous solution of L-1-1 (1.2 g, 6.22 mmol) in dry DCM at 0° C. under N₂ atmosphere was treated with TPP (2.45 g, 9.34 mmol) and stirred at 0° C. for 5 minutes. NBS (1.66 g, 9.34 mmol) wad added and mixture was stirred at 0° C. for 20 minutes and warmed to room temperature for 1 hrs. The reaction was quenched with

H₂O (70 mL) and extracted with DCM (80 mL). The organic layer was dried over anhydrous Na₂SO₄, filtered, and concentrated in vacuo. The residue was purified by flash chromatography (DCM: MeOH=100: 1 to 200: 1) to give the title compound L-1 (1.3 g, 80%, purity 70%) as a liquid.

EI-MS m/z: 251 (M⁺+1).

### Example 2. Preparation of Compound L-2

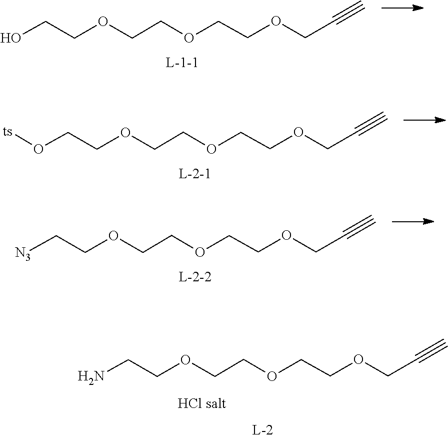

Preparation of Compound L-2-1

A homogeneous solution of L-1-1 (2.88 g, 15.3 mmol) in dry DCM at 0° C. under N₂ atmosphere was treated with p-TsC1 (2.92 g, 15.3 mmol), KOH (3.43 g, 61.2 mmol) and warmed to room temperature for 3.5 hrs. The reaction was quenched with H₂O (50 mL) and extracted with DCM (80 mL×3). The organic layer was dried over anhydrous Na₂SO₄, filtered, and concentrated in vacuo to give the title compound L-2-1 (crude) as a liquid.

¹H NMR (400 MHz, CDCl₃) δ 7.79 (d, J=7.2 Hz, 2H), 7.33 (d, J=7.6 Hz, 2H), 4.20-4.12 (m, 4H), 3.68-3.58 (m, 10H), 2.44 (s, 3H), 2.42 (m, 1H). EI-MS m/z: 343 (M⁺+1).

Preparation of Compound L-2-2

A homogeneous solution of L-2-1 (5.24 g, 15.3 mmol) in anhydrous DMF at room temperature under N₂ atmosphere was treated with NaN₃ (1.49 g, 22.95 mmol) and heated to 60° C. overnight. The reaction was quenched with H₂O (100 mL) and extracted with EA (120 mL×2). The organic layer was washed with brine (150 mL), dried over anhydrous Na₂SO₄, filtered, and concentrated in vacuo. The residue was purified by flash chromatography (EA: HEX=1: 3) to give the title compound L-2-2 (2.39 g, 73% over 2 steps yield) as a liquid.

¹H NMR (400 MHz, CDCl₃) δ 4.19 (d, J=2 Hz, 2H), 3.69-3.56 (m, 10H), 3.38 (m, 2H), 2.42 (m, 1H). ELMS m/z: 236 (M⁺+Na).

Preparation of Compound L-2

A clear solution of L-2-2 (2.39 g, 11.2 mmol) in EA, diethyl ether and 5% HCl at 0° C. under N₂ atmosphere was treated with TPP (2.94 g, 11.2 mmol) and slowly warmed to room temperature overnight. The reaction mixture washed with diethyl ether (50 mL×2) and the H₂O layer was concentrated in vacuo. The liquid was dried under high vacuum to give the title compound L-2 (2.13 g, 85%) as a colorless oil.

¹H NMR (400 MHz, DMSO-d6) δ 4.14 (d, J=1.6 Hz, 2H), 3.62-3.50 (m, 10H), 2.97-2.93 (m, 2H), 2.50 (m, 1H).

### Example 3. Preparation of Compounds L-3 and L-4

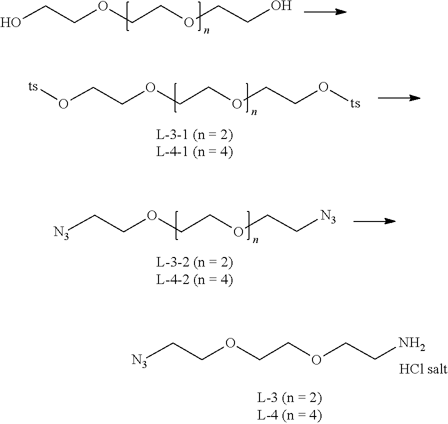

Preparation of Compound L-3-1

A homogeneous solution of tetraethylene glycol (10 g, 51.49 mmol) in dry DCM at 0° C. under N₂ atmosphere was treated with KOH (23.1 g, 411.88 mmol), p-TsC1 (19.6 g, 102.97 mmol) and warmed to room temperature for 2.5 hrs. The reaction was diluted with H₂O (200 mL) and extracted with DCM (250 mL×3). The organic layer was dried over anhydrous Na₂SO₄, filtered, and concentrated in vacuo to give the title compound: L-3-1 (crude) as a liquid.

¹H NMR (400 MHz, CDCl₃) δ 7.78 (d, J=7.6 Hz, 2H), 7.33 (d, J=7.6 Hz, 2H), 4.16-4.13 (m, 4H), 3.68-3.65 (m, 4H), 3.59-3.52 (m, 8H), 2.43 (s, 6H); EI-MS m/z: 503 (M⁺+1).

Preparation of Compound L-3-2

A homogeneous solution of L-3-1 (25.9 g, 51.49 mmol) in anhydrous DMF at room temperature under N₂ atmosphere was treated with NaN₃ (10 g, 154.46 mmol and heated to 60° C. overnight. The reaction was quenched with H₂O (250 mL) and extracted with EA (250 mL×3). The organic layer was washed with brine (350 mL), dried over anhydrous Na₂SO₄, filtered, and concentrated in vacuo. The residue was purified by flash chromatography (EA: HEX=1: 3) to give the title compound: L-3-2 (10.64 g, 85% over 2 steps) as a liquid.

¹H NMR (400 MHz, CDCl₃) δ 3.70-3.66 (m, 12H), 3.41-3.37 (m, 4H); EI-MS m/z: 267 (M⁺+Na).

Preparation of Compound L-3

A clear solution of L-3-2 (10.64 g, 43.56 mmol) in EA, diethyl ether and 5% HCl at 0° C. under N₂ atmosphere was treated with TPP (11.4 g, 43.56 mmol) and slowly warmed to room temperature overnight. The organic layer was concentrated in vacuo. The residue H₂O phase washed with DCM (150 mL×2) and the H₂O layer was concentrated in vacuo. The liquid was dried under high vacuum to give the title compound: L-3 (10.78 g, 78%) as a colorless oil.

EI-MS m/z: 219 (M⁺+1).

Compound L-4 was synthesized via a similar synthetic method of preparing compound L-3.

Preparation of Compound L-4-1

EI-MS m/z: 591 (M⁺+1).

Preparation of Compound L-4-2

Yield 79%, colorless oil.

¹H NMR (600 MHz, CDCl₃) δ 3.69-3.66 (m, 20H), 3.39 (t, J=4.8 Hz, 4H); EI-MS m/z: 355 (M⁺+Na).

Preparation of Compound L-4

Yield 91%, colorless oil.

¹H NMR (600 MHz, CDCl₃) δ 8.15 (br s, 2H), 3.93 (t, J=4.2 Hz, 2H), 3.82-3.66 (m, 18H), 3.49-3.46 (m, 2H), 3.21-3.19 (m, 2H). EI-MS m/z: 307 (M⁺+1).

### Example 4. Preparation of Compound L-5

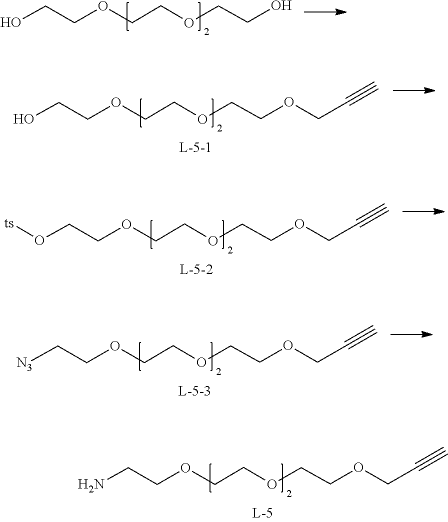

Preparation of Compound L-5-1

A homogeneous solution of tetraethylene glycol (20 g, 102.97 mmol) in dry THF at room temperature under N₂ atmosphere was treated with t-BuOK (54.57 mL, 54.57 mmol) and stirred to room temperature for 30 minutes. Propargyl bromide (6.08 mL, 54.57 mmol) was added dropwise and the resulting mixture was allowed to stand for 15 hours. The reaction mixture was filtered through Celite®, and the Celite® plug was washed with EA (100 mL×2). The filtrate was concentrated under reduced pressure. The residue was purified by flash chromatography (EA: HEX=5: 1) to give the title compound: L-5-1 (10.98 g, 46%) as a liquid.

¹H NMR (400 MHz, CDCl₃) δ 4.20 (d, J=1.6 Hz, 2H), 3.72-3.59 (m, 16H), 2.56 (m, 1H), 2.43 (m, 1H).

Preparation of Compound L-5-2

A homogeneous solution of L-5-1 (10.98 g, 47.27 mmol) in dry DCM at room temperature under N₂ atmosphere was treated with TEA (17.13 mL, 122.90 mmol), p-TsC1 (18.02 g, 94.54 mmol) and stirred to room temperature overnight. The reaction mixture was concentrated in vacuo. The residue was purified by flash chromatography (EA: HEX=1: 3 to 1: 1) to give the title compound: L-5-2 (17.06 g, 93%) as a liquid.

¹H NMR (400 MHz, CDCl₃) δ 7.79-7.77 (m, 2H), 7.33-7.30 (m, 2H), 4.20-4.10 (m, 4H), 3.71-3.55 (m, 14H), 2.43-2.40 (m, 4H).

Preparation of Compound L-5-3

A homogeneous solution of L-5-2 (8.22 g, 21.27 mmol) in anhydrous DMF at room temperature under N₂ atmosphere was treated with NaN₃ (2.07 g, 31.90 mmol) and heated to 60° C. overnight. The reaction was quenched with H₂O (200 mL) and extracted with EA (250 mL×3). The organic layer was washed with brine (350 mL), dried over anhydrous Na₂SO₄, filtered, and concentrated in vacuo. The residue was purified by flash chromatography (EA: HEX=1: 2 to 1: 1) to give the title compound: L-5-3 (2.94 g, 54%) as a liquid.

¹H NMR (400 MHz, CDCl₃) δ 4.20 (d, J=2.4 Hz, 2H), 3.72-3.60 (m, 14H), 3.40-3.37 (m, 2H), 2.42 (m, 1H).

Preparation of Compound L-5

A clear solution of L-5-3 (2.94 g, 11.41 mmol) in EA, diethyl ether and 5% HCl at 0° C. under N₂ atmosphere was treated with TPP (2.99 g, 11.41 mmol) and slowly warmed to room temperature overnight. The organic layer was concentrated in vacuo. The residue H₂O phase washed with DCM (200 mL×2) and the H₂O layer was concentrated in vacuo. The liquid was dried under high vacuum to give the title compound: L-5 (2.55 g, 83%) as a colorless oil.

EI-MS m/z: 232 (M⁺+1).

### Example 5. Preparation of Compound L-6

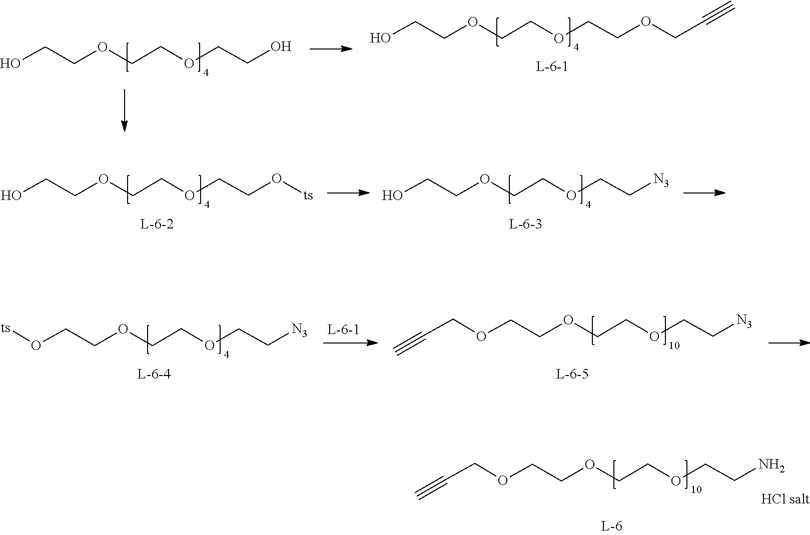

Preparation of Compound L-6-1

A homogeneous solution of hexaethylene glycol (7.8 g, 27.63 mmol) in dry THF at room temperature under N₂ atmosphere was treated with t-BuOK (1.64 g, 14.64 mmol) and stirred to room temperature for 30 minutes. Propargyl bromide (1.63 mL, 14.64 mmol) was added dropwise and the resulting mixture was allowed to stand overnight. The reaction mixture was filtered, and concentrated in vacuo. The residue was purified by flash chromatography (EA: MeOH=97: 3) to give the title compound L-6-1 (4.57 g, 52%) as a liquid.

¹H NMR (400 MHz, CDCl₃) δ 4.20 (d, J=2 Hz, 2H), 3.72-3.59 (m, 24H), 2.65 (m, 1H), 2.42 (m, 1H).

Preparation of Compound L-6-2

A homogeneous solution of hexaethylene glycol (5.6 g, 19.83 mmol) in dry DCM at 0° C. under N₂ atmosphere was treated with Ag₂O (5.52 g, 23.80 mmol), KI (329 mg, 1.98 mmol), p-TsC1 (4.16 g, 21.82 mmol) and stirred to room temperature for 3 hrs. The reaction mixture was filtered, and concentrated in vacuo. The residue was purified by flash chromatography (EA: MeOH=95: 5 to 90: 10) to give the title compound L-6-2 (7.71 g, 89%) as a liquid.

¹H NMR (400 MHz, CDCl₃) δ 7.79 (d, J=6.8 Hz, 2H), 7.33 (d, J=7.6 Hz, 2H), 4.16-4.14 (m, 2H), 3.72-3.57 (m, 22H), 2.65-2.63 (m, 1H), 2.44 (s, 3H).

### Preparation of Compound L-6-3

A homogeneous solution of L-6-2 (7.71 g, 17.66 mmol) in anhydrous DMF at room temperature under N₂ atmosphere was treated with NaN₃ (1.72 g, 26.48 mmol) and heated to 110° C. for 3.5 hrs. The reaction mixture was concentrated in vacuo and DMF removed under high vacuum. The residue was purified by flash chromatography (EA: MeOH=10: 1) to give the title compound: L-6-3 (4.74 g, 87%) as a liquid.

¹H NMR (400 MHz, CDCl₃) δ 3.74-3.58 (m, 22H), 3.41-3.36 (m, 2H), 2.67-2.62 (m, 1H).

Preparation of Compound L-6-4

A homogeneous solution of L-6-3 (4.74 g, 15.42 mmol) in dry DCM at room temperature under N₂ atmosphere was treated with TEA (5.59 mL, 40.10 mmol), p-TsC1 (5.88 g, 30.84 mmol) and stirred to room temperature overnight. The reaction mixture was concentrated in vacuo. The residue was purified by flash chromatography (EA: HEX=5: 1) to give the title compound: L-6-4 (6.52 g, 92%) as a liquid.

¹H NMR (400 MHz, CDCl₃) δ 7.79 (d, J=8 Hz, 2H), 7.34 (d, J=8 Hz, 2H), 4.15 (t, J=5.2 Hz, 2H), 3.69-3.57 (m, 20H), 3.38 (d, J=5.2 Hz, 2H), 2.44 (s, 3H).

### Preparation of Compound L-6-5

A homogeneous solution of L-6-1 (4.53 mg, 14.14 mmol) in dry THF at 0° C. under N₂ atmosphere was treated with 60% NaH (678 mg, 16.96 mmol) and allowed to stand for 30 minutes. L-6-4 (6.52 g, 14.14 mmol) was added, and the resulting mixture was warmed to room temperature for 7 hrs. 60% NaH (678 mg, 16.96 mmol) was added and allowed to stand overnight. 60% NaH (282.7 mg 7.07 mmol) was added and heated to 40° C. overnight. The reaction was allowed to cool at 0° C., quenched with MeOH (100 mL) and concentrated in vacuo. The residue was purified by flash chromatography (EA: MeOH=90: 10) to give the title compound: L-6-5 (7.341 g, 85%) as a liquid.

¹H NMR (400 MHz, CDCl₃) δ 4.19 (m, 2H), 4.70-4.55 (m, 46H), 0.38 (m, 2H), 2.43 (m, 1H).

EI-MS m/z: 610 (M⁺+1).

Preparation of Compound L-6

A clear solution of L-6-5 (906.7 mg, 1.49 mmol) in EA, diethyl ether and 5% HCl at 0° C. under N₂ atmosphere was treated with TPP (390 mg, 1.49 mmol) and slowly warmed to room temperature overnight. The organic layer was concentrated in vacuo. The residue H₂O phase washed with DCM (60 mL×3) and the H₂O layer was concentrated in vacuo. The liquid was dried under high vacuum to give the title compound: L-6 (495 mg, 54%) as a colorless oil.

EI-MS m/z: 584 (M⁺+1).

### Example 6. Preparation of Compound L-7

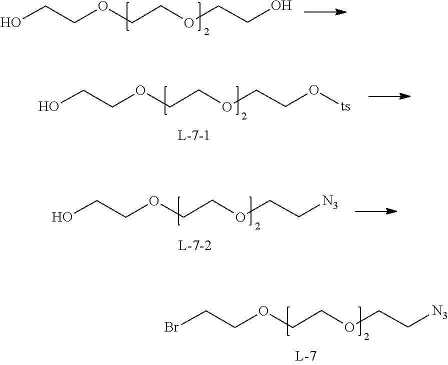

Preparation of Compound L-7-1

A homogeneous solution of tetraethylene glycol (10 g, 51.48 mmol) in dry THF at 0° C. under N₂ atmosphere was treated with NaOH (3 g, 77.22 mmol), p-TsC1 (9.8 g, 51.48 mmol) and allowed to stand for 30 minutes. The reaction mixture was warmed to room temperature for 3 hrs. The reaction was diluted with H₂O (50 mL) and extracted with EA (50 mL×3). The organic layer was dried over anhydrous Na₂SO₄, filtered, and concentrated in vacuo. The residue was purified by flash chromatography (EA: HEX=1: 1 to 5: 1) to give the title compound: L-7-1 (3.15 g, 18%) as a liquid.

¹H NMR (600 Hz, DMSO-d6) δ 7.79 (d, J=8 Hz, 2H), 7.48 (d, J=8.4 Hz, 2H), 4.57 (t, J=5.6 Hz, 1H), 4.12-4.09 (m, 2H), 3.58-3.56 (m, 2H), 3.51-3.44 (m, 10H), 3.42-3.38 (m, 2H), 2.42 (s, 3H); EI-MS m/z: 349 (M⁺+1).

Preparation of Compound L-7-2

A homogeneous solution of L-7-1 (3.15 g, 9.04 mmol) in anhydrous DMF at room temperature under N₂ atmosphere was treated with NaN₃ (3.53 g, 54.24 mmol) and heated to 90° C. overnight. The reaction was quenched with H₂O (30 mL) and extracted with EA (100 mL×3). The organic layer was washed with brine (200 mL), dried over anhydrous Na₂SO₄, filtered, and concentrated in vacuo. The residue was purified by flash chromatography (DCM: MeOH=15: 1) to give the title compound: L-7-2 (1.8 g, 91%) as a liquid.

EI-MS m/z: 220 (M⁺+1).

### Example 7. Preparation of Compound BCN-PNP

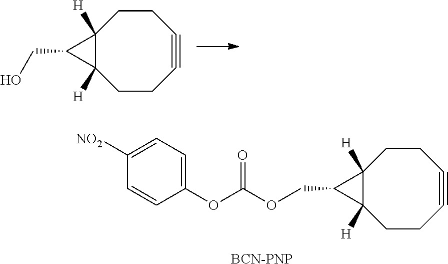

(1R,8S,9s)-Bicyclo[6.1.0]non-4-yn-9-yl methanol (800 mg, 5.3 mmol) was dissolved in DCM (125 mL) at room temperature under N₂ atmosphere. Pyridine (1.22 mL, 15.9 mmol) and 4-nitrophenyl chloroformate (1.75 g, 8.74 mmol) were added thereto. After the mixture was stirred for 4 hours at the same temperature, the reaction was quenched by the addition of saturated NH₄C₁ solution (100 mL) and extracted with EA (100 mL×4). The organic layer was dried over Na₂SO₄, filtered, and concentrated in vacuo. The residue was purified by column chromatography (Hex: EA=10: 1) to obtain compound BCN-PNP (1.34 g, 84%) as white solid.

¹H NMR (600 MHz, CDCl₃) δ 8.29 (d, J=9 Hz, 2H), 7.39 (d, J=9 Hz, 2H), 4.41 (d, J=8.4 Hz, 2H), 2.36-2.24 (m, 6H), 1.62-1.55 (m, 2H), 1.53-1.49 (m, 1H), 1.07 (t, J =10.2 Hz, 2H).

### Example 8. Preparation of Compound L-8

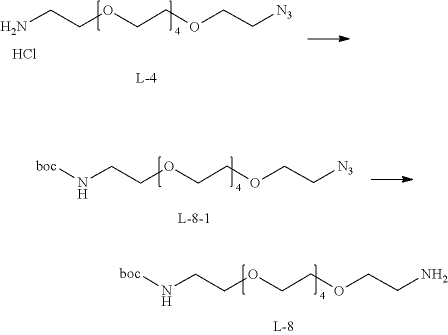

Preparation of Compound L-8-1

A homogeneous solution of L-4 (740 mg, 2.16 mmol) and di-t-butyl dicarbonate (707 mg, 3.24 mmol) in 1,4-dioxane : H₂O=1: 1(10 mL) at room temperature under N₂ atmosphere was treated with NaHCO₃(363 mg, 4.32 mmol) and stirred to room temperature for 2 hours. The reaction was quenched with water (10 mL) and extracted with EA (10 mL×2). The organic layer was dried over anhydrous Na₂SO₄, filtered, and concentrated in vacuo. Compound L-8-1 (809 mg, 96%) was used directly in the next reaction without purification.

EI-MS m/z: 407 (M^{t}).

Preparation of Compound L-8

A homogeneous solution of L-8-1 (809 mg, 1.99 mmol) in anhydrous MeOH (15 mL) at room temperature under H₂ atmosphere was treated with Pd/C (90 mg, 10 wt %) and stirred for 4 hours. The reaction mixture was filtered through Celite® and concentrated in vacuo. Compound L-8 (829 mg, quant) was used directly in the next reaction without purification.

EI-MS m/z: 381 (M^{t}).

### Example 9. Preparation of Compound AMA-1

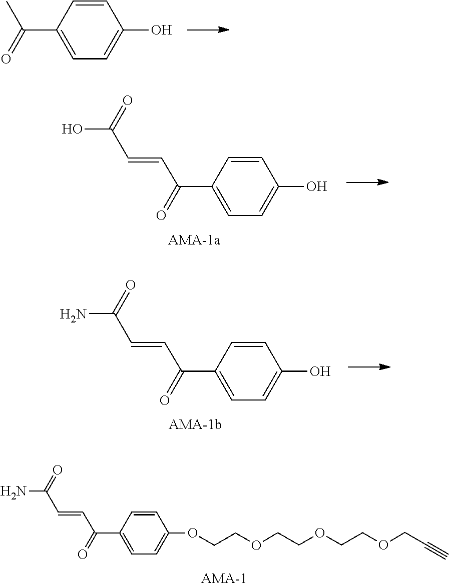

Preparation of Compound AMA-1a

A cloudy mixture of 4-hydroxyacetophenone (5 g, 52.32 mmol) and glyoxylic acid monohydrate (7.4 g, 54.32 mmol) in AcOH at room temperature under N₂ atmosphere was heated to reflux overnight. The reaction was quenched with H₂O (150 mL) and extracted with EA (200 mL×3), dried over anhydrous Na₂SO₄, filtered, and concentrated in vacuo. The residue was purified by flash chromatography (EA: HEX =3: 1 to EA: MeOH=97: 3) to give the title compound AMA-1a(1.17 g, 11%, mixture 6.61 g) as a brown solid.

¹H NMR (400 MHz, DMSO-d6) δ 7.94 (d, J=8 Hz, 2H), 7.86 (d, J=15.6 Hz, 1H), 6.90 (d, J=8.8 Hz, 2H), 6.63 (d, J=15.6 Hz, 1H).

Preparation of Compound AMA-1b

A homogeneous solution of AMA-1a(457.9 mg, 2.38 mmol) in anhydrous DMF at 0° C. under N₂ atmosphere was treated with DMAP (43.7 mg, 0.36 mmol), EDCI (456.8 mg, 2.38 mmol), DIPEA (2.08 mL, 11.91 mmol) and NH₄C₁ (1.25 g, 23.83 mmol), and slowly warmed to room temperature overnight. The reaction was quenched with saturated citric acid (35 mL), saturated NaHCO₃(35 mL) and brine (30 mL) and washed with EA (50 mL), and then dried over anhydrous Na₂SO₄, filtered, and concentrated in vacuo. The residue was purified by flash chromatography (100% DCM to EA: MeOH=97: 3) to give the title compound AMA-1b (24.4 mg, 5%) as a brown solid.

¹H NMR (400 MHz, DMSO-d6) δ 7.94-7.90 (m, 3H), 7.75 (d, J=15.2 Hz, 1H), 7.48 (br s, 1H), 6.88 (d, J=15.2 Hz, 1H), 6.85 (d, J=8.4 Hz, 2H).

### Preparation of Compound AMA-1

A homogeneous solution of AMA-1b (12 mg, 0.063 mmol) and L-1 (31 mg, 0.126 mmol) in anhydrous DMF at 0° C. under N₂ atmosphere was treated with K₂CO₃ (8.7 mg, 0.063 mmol) and heated to 40° C. overnight. The reaction was quenched with H₂O (20 mL) and extracted with EA (25 mL). The organic layer was washed with brine (20 mL), dried over anhydrous Na₂SO₄, filtered, and concentrated in vacuo. The residue was purified by flash chromatography (DCM: MeOH=18: 1) to give the title compound AMA-1 (10 mg, 44%) as a yellowish gum.

¹H NMR (400 MHz, CDCl₃) δ 8.05-7.95 (m, 3H), 7.02-6.94 (m, 3H), 5.73 (br s, 1H), 5.60 (br s, 1H), 4.24-4.19 (m, 4H), 3.92-3.88 (m, 2H), 3.77-3.67 (m, 8H), 2.43-2.42 (m, 1H). ELMS m/z: 362 (M⁺+1).

### Example 10. Preparation of Compound AMA-2 and AMA-3

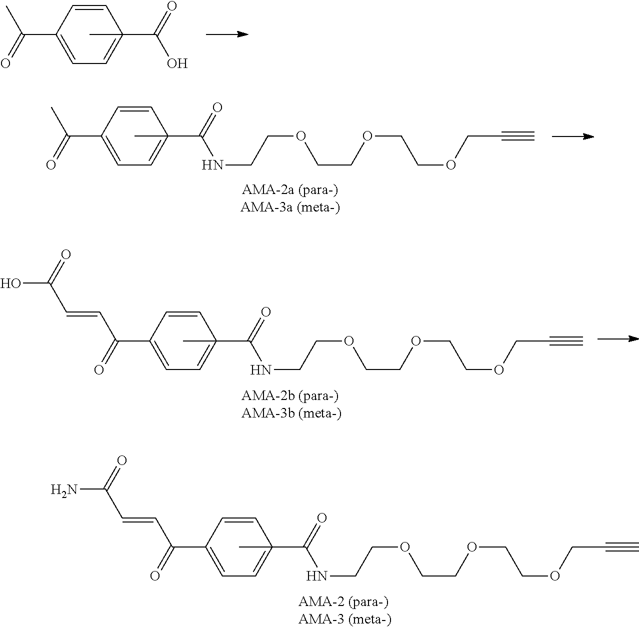

Preparation of Compound AMA-2a

A homogeneous solution of 4-acethylbenzoic acid (306 mg, 1.86 mmol) and L-2 (500 mg, 2.24 mmol) in anhydrous DMF at room temperature under N₂ atmosphere was treated with EDCI (428 mg, 2.24 mmol), DIPEA (0.81 mL, 4.66 mmol), HOBt (342 mg, 2.24 mmol) and stirred to room temperature overnight. The reaction was quenched with saturated citric acid (35 mL). Then the mixture was extracted with saturated NaHCO₃(35 mL), EA (40 mL), and brine (30 mL), and dried over anhydrous Na₂SO₄, filtered, and concentrated in vacuo. The residue was purified by flash chromatography (DCM: MeOH=15: 1) to give the title compound AMA-2a (105 mg, 17%) as a yellowish gum.

¹H NMR (400 MHz, CDCl₃) δ 8.01 (d, J=8.4 Hz, 2H), 7.90 (d, J=8.8 Hz, 2H), 6.90 (br s, 1H), 4.13 (m, 2H), 3.68 (m, 12H), 2.64 (s, 3H), 2.42 (m, 1H). EI-MS m/z: 334 (M⁺+1).

Preparation of Compound AMA-2b

A homogeneous solution of AMA-2a (105 mg, 0.31 mmol) and glyoxylic acid monohydrate (58 mg, 0.63 mmol) in AcOH at room temperature under N₂ atmosphere and heated to reflux for 6.5 hrs. Additional glyoxylic acid monohydrate (58 mg, 0.63 mmol) in AcOH solution was added and the resulting mixture was allowed to stand overnight. The third portion of glyoxylic acid monohydrate (58 mg, 0.63 mmol) in AcOH solution and the resulting mixture was added and stirred 6 hrs. The mixture was concentrated in vacuo. The residue was purified by flash chromatography (DCM : MeOH=15: 1 to 9: 1 to 7: 1) to give the title compound: AMA-2b (39 mg, 32% Yield, 35 mg SM recovered) as a yellowish gum.

EI-MS m/z: 390 (M⁺+1).

Preparation of Compound AMA-2

A homogeneous solution of AMA-2b (14 mg, 0.036 mmol) in dry THF at −15° C. under N₂ atmosphere was treated with NMM (5 μL, 0.043 mmol) and i-BuCO₂C₁ (7 μL, 0.054 mmol) was added dropwise and allowed to stand for 40 minutes. Ammonia 0.5M in THF (1 mL) was added and stirred for 40 minutes. The reaction was quenched with H₂O (10 mL) and extracted with EA (15 mL). The organic layer was washed with brine (10 mL), dried over anhydrous Na₂SO₄, filtered, and concentrated in vacuo. The residue was purified by flash chromatography (DCM: MeOH=9: 1) to give the title compound AMA-2 (11 mg, 79%) as a yellowish gum.

EI-MS m/z: 389 (M⁺+1).

Compound AMA-3 was synthesized via a similar synthetic method of preparing compound AMA-2.

Preparation of Compound AMA-3a

Yield 40%, yellowish gum.

¹H NMR (400 MHz, CDCl₃) δ 8.38 (s, 1H), 8.09 (d, J=7.6 Hz, 1H), 8.03 (d, J=8.4 Hz, 1H), 7.57-7.53 (m, 1H), 6.95 (br s, 1H), 4.12 (m, 2H), 3.70-3.68 (m, 12H), 2.66 (s, 3H), 2.40 (m, 1H). ELMS m/z: 334 (M⁺+1).

Preparation of Compound AMA-3b

Yield 65%, yellowish gum.

EI-MS m/z: 390 (M⁺+1).

Preparation of Compound AMA-3

Yield 36%, yellowish gum.

¹H NMR (400 MHz, CDCl₃) δ 8.47 (s, 1H), 8.14 (d, J=7.6 Hz, 2H), 8.02 (d, J=15.2 Hz, 1H), 7.62-7.58 (m, 1H), 7.40 (br s, 1H), 7.00 (d, J=14.8 Hz, 1H), 6.10 (br s, 1H), 5.91 (br s, 1H), 4.11 (m, 2H), 3.72-3.67 (m, 12H), 2.41 (m, 1H). EI-MS m/z: 389 (M⁺+1).

### Example 11. Preparation of Compound AMA-4

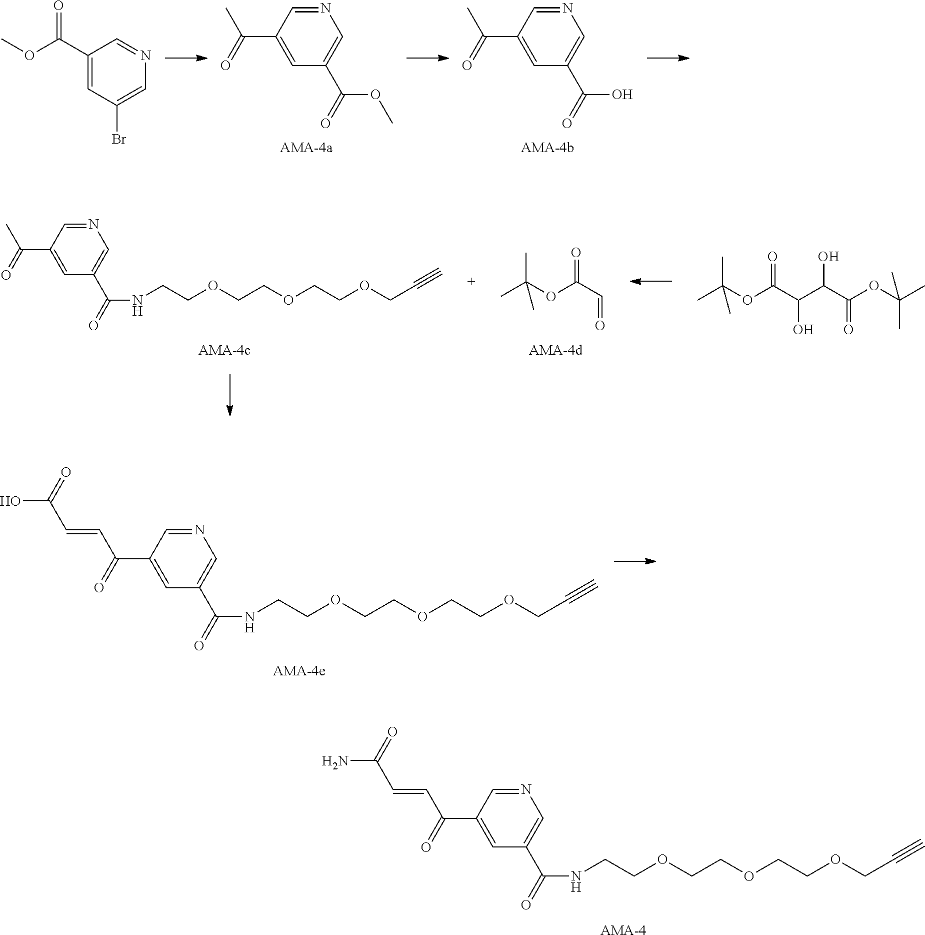

Preparation of Compound AMA-4a

A solution of methyl 5-bromonicotinate (3 g, 13.89 mmol), PdC12(PPh3)₂ (487 mg, 0.96 mmol) and tributyl(1-ethoxyvinyl)tin (5.86 mL, 17.36 mmol) in anhydrous toluene at room temperature under N₂ atmosphere was heated to reflux for 3 hrs. The mixture was filtered through Celite® and washed with MeOH (100 mL). The filtrate was concentrated under reduced pressure. The residue was dissolved in MeOH (30 mL) and 10M HCl (30 mL) at room temperature under N₂ atmosphere was added. The resulting mixture was allowed to stand for 2 hours. The reaction was quenched with saturated Na₂CO₃ (120 mL) and extracted with EA (150 mL×3). The organic layer was washed with brine (250 mL), dried over anhydrous Na₂SO₄, filtered, and concentrated under reduced pressure. The residue was purified by flash chromatography (EA: HEX=1: 2) to obtain compound AMA-4a (2.22 g, 89%) as a white solid.

¹H NMR (400 MHz, CDCl₃) δ 9.37 (s, 1H), 9.31 (s, 1H), 8.79 (s, 1H), 4.00 (s, 3H), 2.69 (s, 3H).

### Preparation of Compound AMA-4b

A homogeneous solution of AMA-4a (636 mg, 3.55 mmol) in MeOH at room temperature under N₂ atmosphere was treated with 1N NaOH (10.64 mL) and stirred for 1 hr. After the mixture was concentrated under reduced pressure, the reaction was quenched with 1N HCl (pH 2) and extracted with EA (80 mL×3). The organic layer was washed with brine (150 mL), dried over anhydrous Na₂SO₄, filtered, and concentrated in vacuo to give the title compound AMA-4b as a white solid. The compound AMA-4b was used directly in the next reaction without purification.

¹H NMR (400 MHz, DMSO-d6) δ 9.31 (s, 1H), 9.25 (s, 1H), 8.64 (s, 1H), 2.69 (s, 3H).

### Preparation of Compound AMA-4c

A homogeneous solution of AMA-4b (606 mg, 3.67 mmol) and L-2 (985 g, 4.40 mmol) in anhydrous DMF at room temperature under N₂ atmosphere was treated with EDCI (1.06 g, 5.50 mmol), DIPEA (1.92 mL, 11.01 mmol), HOBt (843 mg, 5.50 mmol) and stirred to room temperature overnight. The reaction was extracted and washed with EA (100 mL×2) and brine (80 mL), dried over anhydrous Na₂SO₄, filtered, and concentrated in vacuo. The residue was purified by flash chromatography (DCM: MeOH=15: 1) to give the title compound AMA-4c (880 mg, 74% over the 2 step yield) as a yellowish gum.

¹H NMR (400 MHz, CDCl₃) δ 9.22 (s, 1H), 9.19 (s, 1H), 8.61 (s, 1H), 4.08 (m, 2H), 3.67-3.60 (m, 12H), 2.66 (s, 3H), 2.38 (m, 1H). EI-MS m/z: 335 (M⁺+1).

Preparation of Compound AMA-4d

A solution of di-tert-butyl tartrate (500 mg, 1.91 mmol) in MeOH at 0° C. under N₂ atmosphere was treated with NaIO4 (489 mg, 2.29 mmol) in H₂O solution and allowed to stand for 1.5 hrs. The reaction was quenched with H₂O (40 mL) and extracted with diethyl ether (45 mL×3), dried over anhydrous Na₂SO₄, filtered, and concentrated in vacuo to give the title compound AMA-4d as a colorless oil. The compound AMA-4d was used directly in the next reaction without purification.

Preparation of Compound AMA-4e

A homogeneous solution of AMA-4c (48 mg, 0.14 mmol) and AMA-4d (56 mg, 0.43 mmol) in AcOH at room temperature under N₂ atmosphere was heated to reflux overnight. The mixture was concentrated in vacuo. The residue was purified by flash chromatography (DCM: MeOH=15: 1 to 9: 1 to 7: 1) to give the title compound AMA-4e (27 mg, 48%) as a yellowish gum.

EI-MS m/z: 391 (M⁺+1).

Preparation of Compound AMA-4

A homogeneous solution of AMA-4e (27 mg, 0.069 mmol) in dry THF at −15° C. under N₂ atmosphere was treated with NMM (9.1 uL, 0.083 mmol) and i-BuCO₂C₁ (13.5 μL, 0.104 mmol) was added dropwise and stirred for 40 minutes. 0.5M Ammonia in THF (2 mL) was added and the resulting mixture was stirred for 30 minutes. The reaction was quenched with H₂O (30 mL) and extracted with EA (35 mL). The organic layer was washed with brine (30 mL), dried over anhydrous

Na₂SO₄, filtered, and concentrated in vacuo. The residue was purified by flash chromatography (DCM: MeOH=15: 1) and prep-HPLC to give the title compound AMA-4 (1.2 mg, 4%) as a yellowish gum.

EI-MS m/z: 390 (M⁺+1).

### Example 12. Preparation of Compound pyrMPS-1

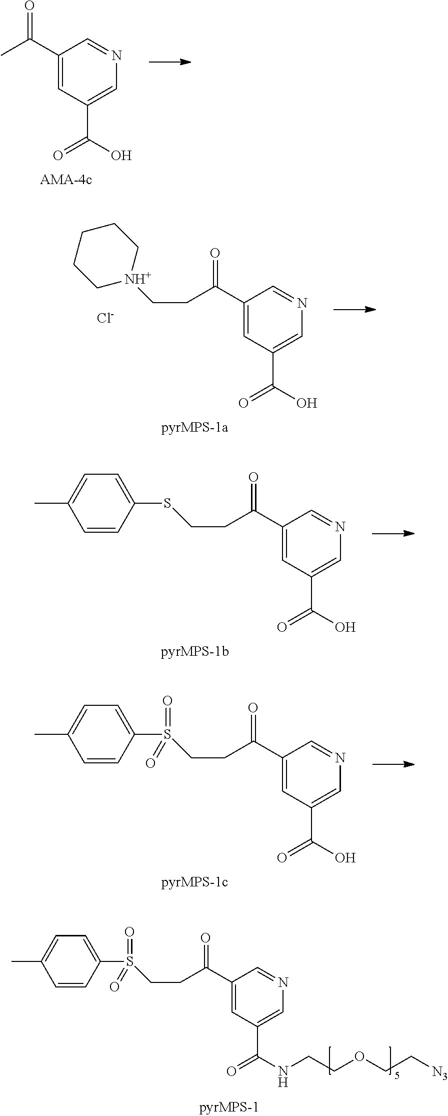

Preparation of Compound pyrMPS-1 a

A cloudy mixture of AMA-4c (2.67 g, 16.19 mmol), piperidine HCl (1.97 g, 16.19 mmol) and paraformaldehyde (1.46 g, 48.57 mmol) in EtOH (25 mL) at room temperature under N₂ atmosphere was treated with conc. HCl (178 μL) and heated to reflux overnight. After the reaction was allowed to cool to room temperature, the mixture was quenched with acetone (25 mL) and cooled to 0° C. and stirred for 1 hr. The solid were collected by filtration, washed with diethyl ether (50 mL×2) and dried under high vacuum to give the title compound pyrMPS-1a (1.45 g, 30%, mixture 3.21 g) as white solid.

EI-MS m/z: 264 (M⁺+1).

Preparation of Compound pyrMPS-1b

A cloudy mixture of pyrMPS-1 a (1.45 g, 4.85 mmol) and 4-methylbenzenethiol (603 mg, 4.85 mmol) in EtOH (15 mL) and MeOH (10 mL) at room temperature under N₂ atmosphere was treated with piperidine (72 0.73 mmol) and heated to reflux overnight. The reaction was allowed to cool to 0° C. for 1 hr. The solids were collected by filtration, washed with diethyl ether (50 mL×2) and dried under high vacuum to give the title compound Int-5-3 (154 mg, 11%, mixture 1.31 g) as white solid.

¹H NMR (400 Hz, DMSO-d6) δ 9.26 (d, J=2 Hz, 1H), 9.23 (d, J=2 Hz, 1H), 8.58 (m, 1H), 7.26 (d, J=8 Hz, 2H), 7.13 (d, J=8.4 Hz, 2H), 3.46 (t, J=7.2 Hz, 2H), 3.24 (d, J=7.2 Hz, 2H), 2.26 (s, 3H). EI-MS m/z: 302 (M⁺+1).

Preparation of Compound pyrMPS-1c

A cloudy mixture of pyrMPS-1b (154 mg, 0.51 mmol) in MeOH (8 mL) and H₂O (8 mL) at 0° C. under N₂ atmosphere was treated with oxone (691 mg, 1.12 mmol) and warmed to room temperature for 6.5 hrs. The reaction was quenched with H₂O (30 mL) and extracted with CHCl₃ (50 mL×4). The organic layer was washed with brine (150 mL), dried over anhydrous Na₂SO₄, filtered, and concentrated in vacuo to give the title compound pyrMPS-1c (74 g, 43%) as white solid.

EI-MS m/z: 334 (M⁺+1).

Preparation of Compound pyrMPS-1

A homogeneous solution of pyrMPS-1c (74 mg, 0.22 mmol) and L-4 (91 mg, 0.27 mmol) in anhydrous DMF (5 mL) at room temperature under N₂ atmosphere was treated with HBTU (106 mg, 0.27 mmol), DIPEA (77.4 μL, 0.44 mmol) and stirred at room temperature for 3 hrs. The reaction was quenched with H₂O (40 mL) and extracted with DCM (50 mL×4). The organic layer was washed with brine (150 mL), dried over anhydrous Na₂SO₄, filtered, and concentrated in vacuo. The residue was purified by prep-HPLC to give the title compound pyrMPS-1 (18 mg, 13%, mixture 43 mg) as yellowish gum.

EI-MS m/z: 622 (M⁺+1).

### Example 13. Preparation of Compound AMA-5

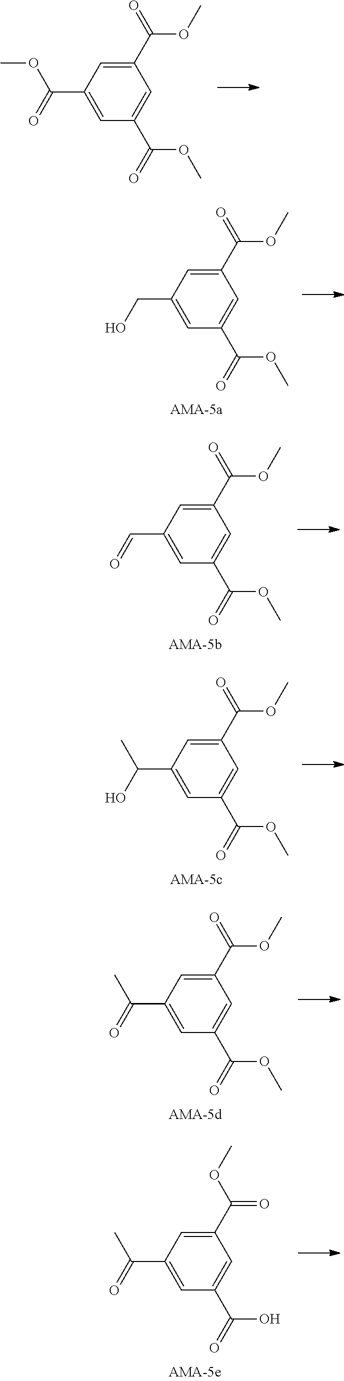

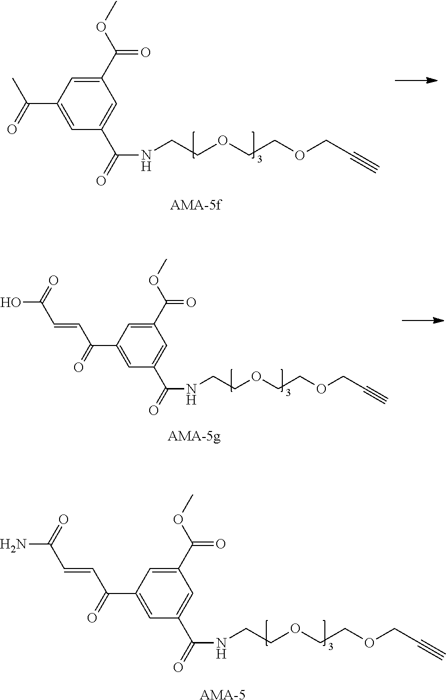

Preparation of Compound AMA-5a

A homogeneous solution of trimethyl-1,3,5-benzenetricarboxylate (34.28 g, 135.91 mmol) in dry THF at 0° C. under N₂ atmosphere was treated with 4M LiBH4 in THF (16.99 mL, 67.95 mmol) and heated to reflux overnight. After the reaction was cooled to room temperature, the mixture was acidified with 4N HCl (pH 2) and quenched with H₂O (800 mL). The mixture was extracted with EA (800 mL×2). The organic layer was dried over anhydrous Na₂SO₄, filtered, and concentrated in vacuo. The residue was purified by flash chromatography (EA: HEX=1: 5 to 1: 2) to give title compound AMA-5a (16.75 g, 55%, 6.07 g Sm recovered) as a white solid.

¹H NMR (400 MHz, CDCl₃) δ 8.59 (s, 1H), 8.23 (s, 2H), 4.81 (d, J=6 Hz, 2H), 3.95 (s, 6H), 1.97 (t, J=5.6 Hz, 1H).

Preparation of Compound AMA-5b

A homogeneous solution of AMA-5a (16.75 g, 74.72 mmol) in dry DCM at room temperature under N₂ atmosphere was treated with DCC (80.53 g, 373.58 mmol) and heated to reflux overnight. The reaction mixture was purified by flash chromatography (EA: HEX=1: 3) to give title compound AMA-5b (13.84 g, 83%) as a white solid.

¹H NMR (400 MHz, CDCl₃) δ 10.13 (s, 1H), 8.92 (s, 1H), 8.72 (s, 2H), 4.00 (s, 6H).

Preparation of Compound AMA-5c

A homogeneous solution of AMA-5b (13.84 g, 62.30 mmol) in dry THF at 0° C. under N₂ atmosphere was treated with 3M MeMgBr in diethyl ether (20.77 mL, 62.30 mmol) and stirred for 2.5 hrs. After the reaction was quenched with saturated NH₄C₁ (150 mL), the mixture was extracted with EA (200 mL). The organic layer was washed with brine (150 mL), dried over anhydrous Na₂SO₄, filtered, and concentrated in vacuo. The residue was purified by flash chromatography (EA: HEX=1: 3 to 1: 2) to give title compound AMA-5c (6.6 g, 44%) as a white solid.

¹H NMR (400 MHz, CDCl₃) δ 8.57 (s, 1H), 8.24 (s, 2H), 5.05-4.99 (m, 1H), 3.95 (s, 6H), 2.01 (d, J=3.6 Hz, 1H), 1.53 (t, J=6.8 Hz, 3H).

### Preparation of Compound AMA-5d

A homogeneous solution of AMA-5c (6.6 g, 27.70 mmol) in dry DCM at room temperature under N₂ atmosphere was treated with DCC (29.86 g, 138.52 mmol) and heated to reflux overnight. The reaction mixture was purified by flash chromatography (EA: HEX=1: 5) to give title compound AMA-5d (4.75 g, 73%, mixture 1.25 g) as a white solid.

¹H NMR (400 MHz, CDCl₃) δ 8.87 (m, 1H), 8.78 (m, 2H), 4.00 (s, 6H), 2.70 (s, 3H).

### Preparation of Compound AMA-5e

A homogeneous solution of AMA-5d (4.75 g, 20.11 mmol) in acetone at 0° C. under N₂ atmosphere was treated with NaOH in MeOH solution (845 mg, 21.11 mmol) and warmed to room temperature. After the reaction mixture was stirred overnight, the mixture was concentrated in vacuo and dried under high vacuum. The residue was dissolved in H₂O and acidified with 4N HCl (pH 1-2). The H₂O phase was extracted with EA (100 mL×2) and dried over anhydrous Na₂SO₄, filtered, and concentrated in vacuo. The residue was purified by flash chromatography (DCM: MeOH=20: 1) to give title compound AMA-5e (2.34 g, 52%, mixture 1.72 g) as a yellowish solid.

¹H NMR (400 MHz, CDCl₃) δ 8.94 (s, 1H), 8.85 (s, 1H), 8.83 (s, 1H), 4.00 (s, 3H), 2.72 (s, 3H).

### Preparation of Compound AMA-5f

A homogeneous solution of AMA-5e (200 mg, 0.9 mmol) and L-5 (337 mg, 1.26 mmol) in dry DCM at 0° C. under N₂ atmosphere was treated with EDCI (259 mg, 1.35 mmol), TEA (376 uL, 2.7 mmol), HOBt (207 mg, 1.35 mmol) and warmed to room temperature. After the reaction mixture was stirred overnight, the reaction was quenched with H₂O (30 mL) and extracted with DCMC (35 mL×6). The organic layer was washed with brine (150 mL), dried over anhydrous Na₂SO₄, filtered, and concentrated in vacuo. The residue was purified by flash chromatography (EA=100%) to give the title compound AMA-5f (248 mg, 63%) as a yellowish oil.

¹H NMR (400 MHz, CDCl₃) δ 8.71 (s, 1H), 8.66 (s, 1H), 8.61 (s, 1H), 7.17 (br s, 1H), 4.13 (d, J=2.4 Hz, 2H), 3.98 (s, 3H), 3.71-3.63 (m, 16H), 2.70 (s, 3H), 2.40 (m, 1H).

Preparation of Compound AMA-5g

A homogeneous solution of AMA-5f (248 mg, 0.57 mmol) and glyoxylic acid monohydrate (410 mg, 4.56 mmol) in AcOH at room temperature under N₂ atmosphere was heated to reflux for 8 hrs. Five more portions of glyoxylic acid monohydrate (210 mg, 2.28 mmol) in AcOH were added to the reaction mixture at intervals of 4 hours. After the mixture was concentrated in vacuo, the residue was purified by flash chromatography (EA=100% to DCM: MeOH=9: 1) to give the title compound AMA-5g (124 mg, 44%) as a yellowish oil.

EI-MS m/z: 492 (M⁺+1).

Preparation of Compound AMA-5

A homogeneous solution of AMA-5g (124 mg, 0.25 mmol) in dry THF at −15° C. under N₂ atmosphere was treated with NMM (33 μLL, 0.30 mmol) and i-BuCO₂C₁ (49 μL, 0.38 mmol) was added dropwise and stirred for 40 minutes. After 0.5M Ammonia in THF (1 mL) was added thereto, the mixture was stirred for 30 minutes. The reaction mixture was concentrated in vacuo. The residue was dissolved in DMSO and acidified with acetic acid. The mixture was purified by prep-HPLC to give the title compound AMA-6 (28 mg, 23%) as a yellowish gum.

¹H NMR (400 MHz, CDCl₃) δ 8.77 (s, 1H), 8.75 (s, 1H), 8.70 (s, 1H), 8.03 (d, J=15.6 Hz, 1H), 7.79 (br s, 1H), 7.03 (d, J=15.6 Hz, 1H), 6.40 (br s, 1H), 6.13 (br s, 1H), 4.12-4.09 (m, 2H), 3.96 (s, 3H), 3.75-3.61 (m, 16H), 2.39 (m, 1H). EI-MS m/z: 491 (M⁺+1).

### Example 14. Preparation of Compound AMA-6

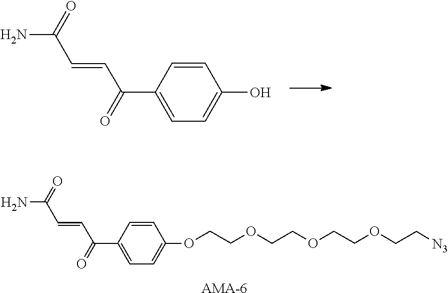

A homogeneous solution of phenol (24.9 mg, 0.13 mmol) and L-7 (43.4 mg, 0.15 mmol) in anhydrous DMF at 0° C. under N₂ atmosphere was treated with K₂CO₃ (27 mg, 0.20 mmol) and warmed to room temperature. After the reaction mixture was stirred for 2.5 hrs, a second portion of K₂CO₃ (9 mg, 0.07 mmol) was added to the reaction mixtur, and the mixture was stirred at 40° C. overnight. The reaction was quenched with H₂O (15 mL) and extracted with DCM (20 mL). The organic layer was dried over anhydrous Na₂SO₄, filtered, and concentrated in vacuo. The residue was purified by flash chromatography (DCM: MeOH=15: 1) and prep-HPLC to give the title compound AMA-6 (5.9 mg, 12%) as a yellowish oil.

EI-MS m/z: 393 (M⁺+1).

### Example 15. Preparation of Compound AMA-7

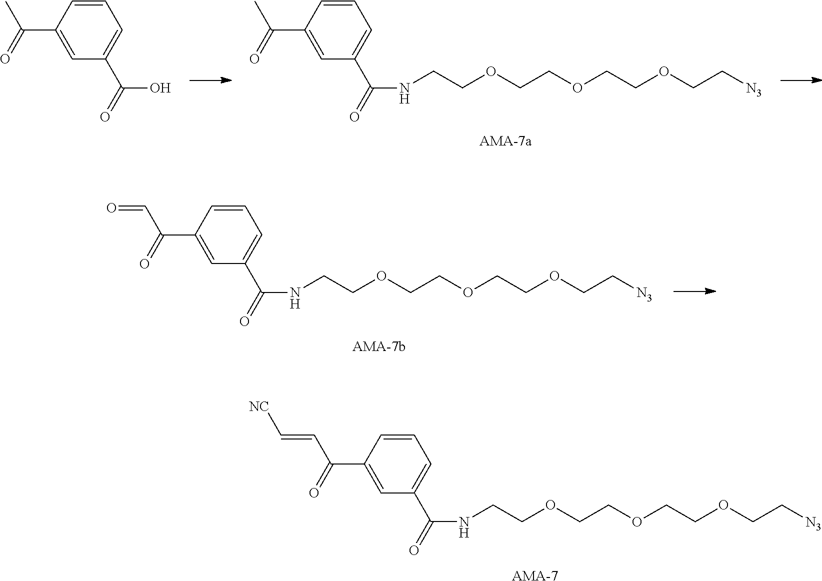

Preparation of Compound AMA-7a

A homogeneous solution of 3-acetyl benzoic acid (5 g, 30.46 mmol) and L-3 (7.76 g, 30.46 mmol) in anhydrous DMF at 0° C. under N₂ atmosphere was treated with TBTU (19.56 g, 60.92 mmol), TEA (21.2 mL, 152.3 mmol). The reaction was warmed to room temperature and stirred overnight. The mixture was extracted with

EA (500 mL), 1N HCl (350 mL), and saturated NaHCO₃(350 mL). The organic layer was washed with brine (350 mL), dried over anhydrous Na₂SO₄, filtered, and concentrated in vacuo. The residue was dried under high vacuum to give the title compound AMA-7a (7.77 g, 70%) as a dark brown oil.

¹H NMR (400 MHz, CDCl₃) δ 8.38-8.37 (m, 1H), 8.10-8.07 (m, 1H), 8.05-8.02 (m, 1H), 7.57-7.53 (m, 1H), 6.94 (br s, 1H), 3.71-3.61 (m, 14H), 3.34 (d, J=5.2 Hz, 2H), 2.66 (s, 3H). EI-MS m/z: 365 (M⁺+1).

Preparation of Compound AMA-7b

A solution of SeO₂ (365.4 mg, 3.29 mmol) in 1,4-dioxane and H₂O at room temperature under N₂ atmosphere was heated to 50° C. for 30 minutes. AMA-7a (300 mg, 0.82 mmol) in 1,4-dioxane and H₂O solution was slowly added thereto. The reaction mixture was refluxed for 4 hrs. Additional SeO₂ (182.7 mg, 1.65 mmol) in 1,4-dioxane and H₂O solution was added, and the resulting mixture was and the resulting mixture was stirred overnight. After cooling to room temperature, the mixture was filtered through Celite® and concentrated in vacuo. The residue was extracted with DCM (50 mL×2) and washed with H₂O (35 mL). The organic layer was dried over anhydrous Na₂SO₄, filtered, and concentrated in vacuo. The residue was dried under high vacuum to give the title compound AMA-7b (crude) as a yellowish oil.

EI-MS m/z: 397 (M^{t}+H₂O).

Preparation of Compound AMA-7

A homogeneous solution of AMA-7b (97.6 mg, 0.26 mmol) in dry DCM at room temperature under N₂ atmosphere was treated with (triphenylphosphoranylidene)acetonitrile (77.7 mg, 0.26 mmol) and stirred overnight. The reaction was quenched with H₂O (20 mL) and extracted with DCM (30 mL). The organic layer was dried over anhydrous Na₂SO₄, filtered, and concentrated in vacuo. The residue was purified by flash chromatography (EA: HEX=2: 1 to 3: 1 to 5: 1 to DCM: MeOH=20: 1) and prep-HPLC to give the title compound AMA-7 (9.5 mg, 9%) as a white gum.

EI-MS m/z: 402 (M^{t}+1).

### Example 16. Preparation of Compound AMA-8

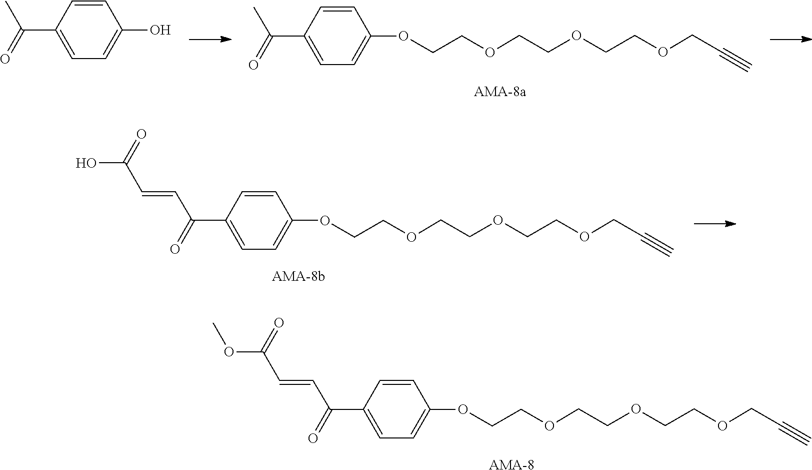

Preparation of Compound AMA-8a

A homogeneous solution of 4-hydroxyacetophenone (87 mg, 0.64 mmol) and L-1 (242 mg, 0.96 mmol) in anhydrous DMF (3 mL) at room temperature under N₂ atmosphere was treated with K₂CO₃ (177 mg, 1.28 mmol) and stirred at room temperature overnight. The reaction was quenched with water (10 mL) and extracted with EA (10 mL×2). The organic layer was washed with brine (15 mL), dried over anhydrous Na₂SO₄, filtered, and concentrated in vacuo. The residue was purified by flash chromatography (EA: Hex=1: 2) to give the title compound AMA-8a (120 mg, 62%).

EI-MS m/z: 307 (M^{t}).

Preparation of Compound AMA-8b

A homogeneous solution of AMA-8a (120 mg, 0.39 mmol) in AcOH (4 mL) under N₂ atmosphere was treated with glyoxylic acid monohydrate (47 mg, 0.51 mmol). The mixture was refluxed overnight. Five more portions of glyoxylic acid monohydrate (210 mg, 2.28 mmol) in AcOH were added to the reaction mixture at intervals of 4 hours. After the mixture was concentrated in vacuo, the residue was purified by flash chromatography (DCM: MeOH=12: 1) to give the title compound AMA-8b (28 mg, 20%) as a yellowish gum.

EI-MS m/z: 363 (M^{t}).

Preparation of Compound AMA-8

A homogeneous solution of AMA-8b (24 mg, 0.08 mmol) and iodomethane (14 μL, 0.23 mmol) in anhydrous DMF (2 mL) at room temperature under N₂ atmosphere was treated with K₂CO₃ (21 mg, 0.15 mmol). The reaction mixture was stirred to room temperature for 3 hours. The reaction was quenched by addition of water (5 mL) and extracted with EA (5 mL×2). The organic layer was washed with brine (8 mL), dried over anhydrous Na₂SO₄, filtered, and concentrated in vacuo. The residue was purified by flash chromatography (EA: Hex=1: 1) to give the title compound AMA-8 (16 mg, 65%).

¹H-NMR (400 MHz, CDCl₃) δ 8.00 (d, J=8.8 Hz, 2H), 7.94 (d, J=15.6 Hz, 1H), 7.01 (d, J=8.8 Hz, 2H), 6.88 (d, J=15.6 Hz, 1H), 4.25-4.22 (m, 4H), 3.92-3.89 (m, 2H), 3.86 (s, 3H), 3.76-3.75 (m, 2H), 3.71 (s, 6H), 2.44 (s, 1H); EI-MS m/z: 377 (M^{t}).

### Example 17. Preparation of Compound AMA-9

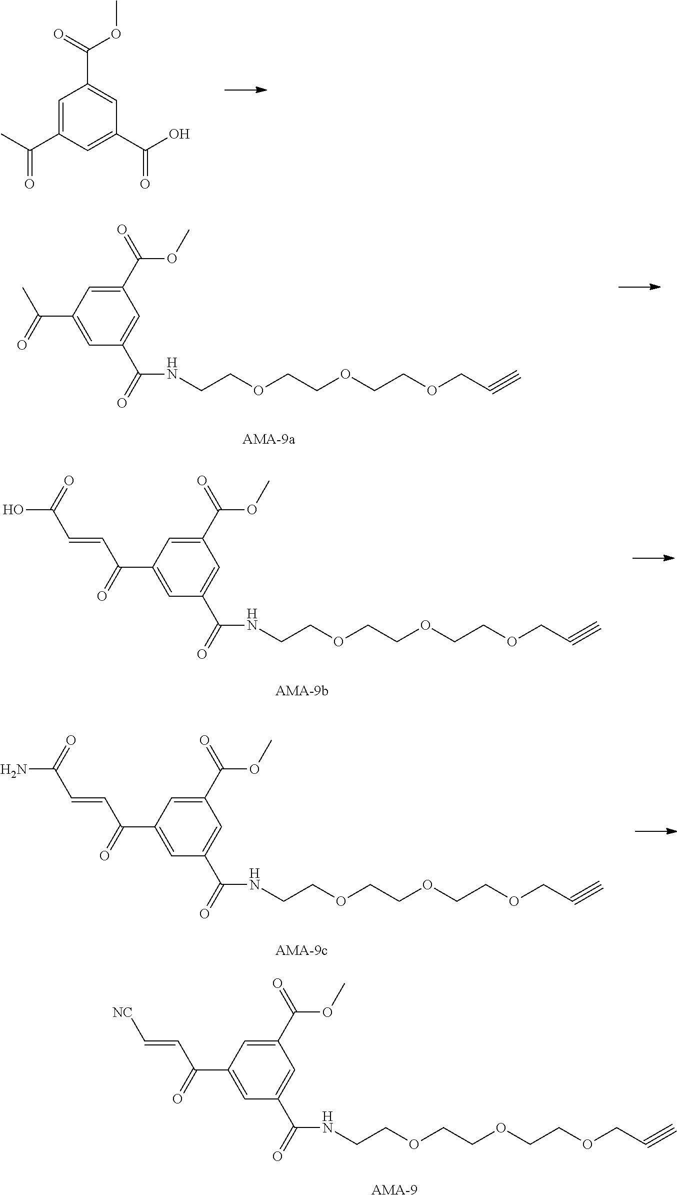

Compound AMA-9a, AMA-9b, and AMA-9c were synthesized via a similar manner to the preparation method of the compound AMA-5 of Example 13.

Preparation of Compound AMA-9a

Yield 77%. ¹H NMR (600 MHz, CDCl₃) δ 8.71 (s, 1H), 8.65 (s, 1H), 8.60 (s, 1H), 7.05 (s, 1H), 4.12 (s, 2H), 3.99 (s, 3H), 3.72-3.69 (m, 12H), 2.70 (s, 3H), 2.38 (s, 1H); EI-MS m/z: 392 (M^{t}).

Preparation of Compound AMA-9b

Yield 27%. EI-MS m/z: 448 (M^{t}).

Preparation of Compound AMA-9c

Yield 27%. EI-MS m/z: 447 (M^{t}).

Compound AMA-9 was synthesized via a similar manner to the preparation method of the compound AMA-7 of Example 15.

Yield 8%. EI-MS m/z: 429 (M^{t}).

### Example 18. Preparation of Compound AMA-10

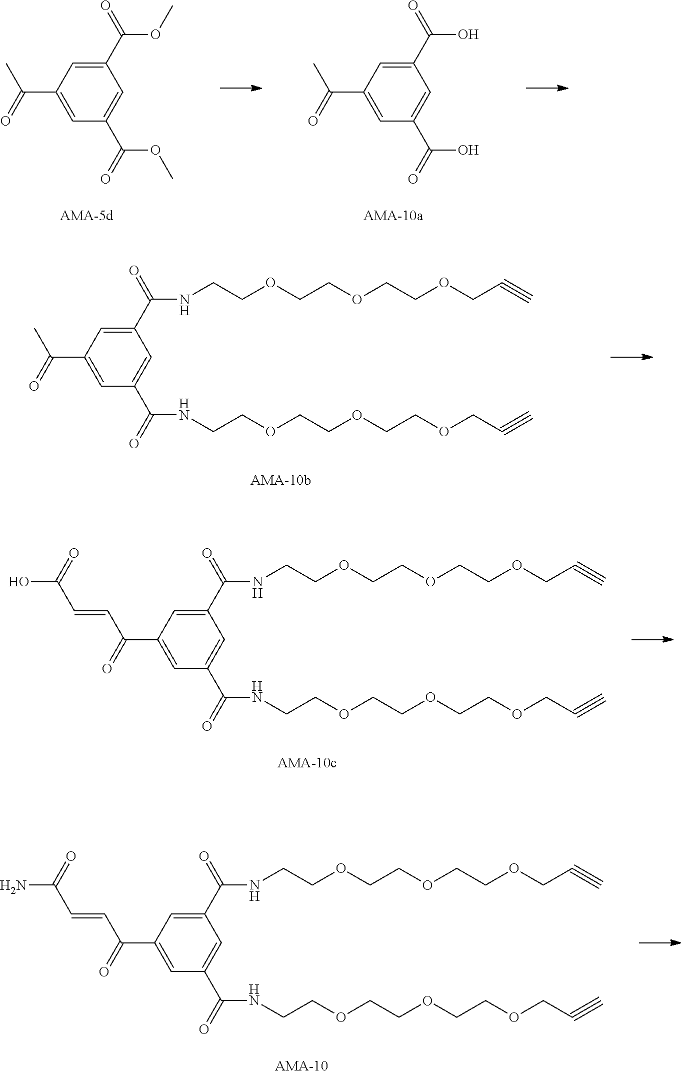

Preparation of Compound AMA-10a

A homogeneous solution of AMA-5d (1.05 g, 4.44 mmol) in anhydrous MeOH (50 mL) at 0° C. under N₂ atmosphere was treated with NaOH (356 mg, 8.88 mmol). The mixture was warmed up to room temperature and refluxed overnight. The mixture was quenched by cautious addition of diluted HCl (10 mL) and concentrated under reduced pressure. The aqueous layer was subjected to extraction with EA (20 mL), and the obtained organic layer was dried over anhydrous Na₂SO₄, filtered, and concentrated in vacuo. After concentration, compound AMA-10a (984 mg, 100%) was used directly in the next reaction without purification.

EI-MS m/z: 209 (M^{t}).

Compound AMA-10b, AMA-10c, and AMA-10 were synthesized via a similar manner to the preparation method of the compound AMA-5 of Example 13.

Preparation of Compound AMA-10b

Yield 84%.

EI-MS m/z: 547 (M^{t}).

Preparation of Compound AMA-10c

Yield 22%.

EI-MS m/z: 603 (M^{t}).

Preparation of Compound AMA-10c

Yield 32%.

EI-MS m/z: 602 (M^{t}).

### Example 19. Preparation of Compound pyrMPS-2 and PyrMPS-3

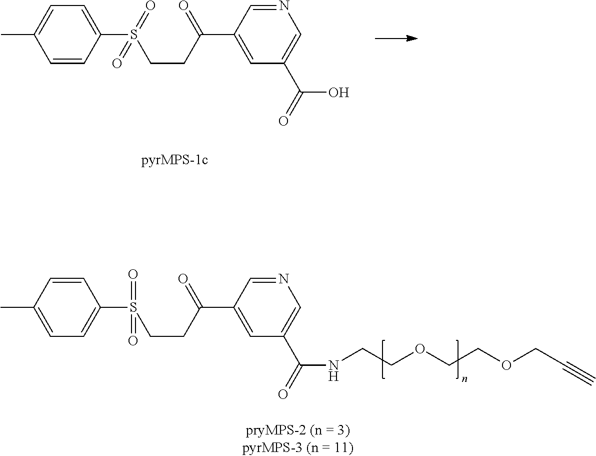

Compound pyrMPS-2 and pyrMPS-3 were synthesized via a similar manner to the preparation method of the compound pyrMPS-1 of Example 12.

Preparation of Compound pyrMPS-2

Yield 22%.

EI-MS m/z: 547 (M^{t}).

Preparation of Compound pyrMPS-3

Yield 26%.

EI-MS m/z: 900 (M^{t}).

### Example 20. Preparation of Compound mMPS-1 and mMPS-2

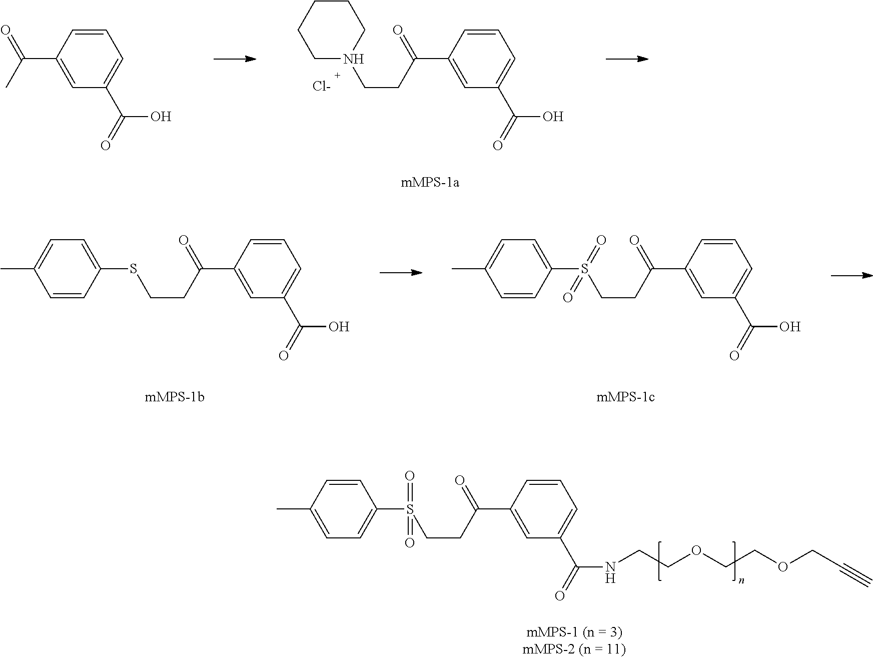

Compound mMPS-1 and mMPS-2 were synthesized via a similar manner to the preparation method of the compound pyrMPS-1 of Example 12.

Preparation of Compound mMPS-1a

Yield 51%, white solid.

EI-MS m/z: 262 (M^{t}).

Preparation of Compound mMPS-1b

Yield 72%, white solid

¹H NMR (600 Hz, DMSO-d6) δ 8.40 (s, 1H), 8.18-8.15 (m, 2H), 7.66-7.63 (m, 1H), 7.26 (d, J=7.8 Hz, 2H), 7.14 (d, J=7.8 Hz, 2H), 3.40-3.37 (m, 2H), 3.26-3.23 (m, 2H), 2.27 (s, 3H); EI-MS m/z: 301 (M⁺+1).

Preparation of Compound mMPS-1c

Yield 47%, yellowish solid.

¹H NMR (600 Hz, DMSO-d6) δ 8.37-8.36 (m, 1H), 8.20-8.16 (m, 2H), 7.81 (d, J=7.8 Hz, 2H), 7.68-7.65 (m, 1H), 7.46 (d, J=8.4 Hz, 2H), 3.66-3.63 (m, 2H), 3.44-3.41 (m, 2H), 2.41 (s, 3H); EI-MS m/z: 333 (M^{t}+1).

Preparation of Compound mMPS-1

Yield 60%, white solid.

EI-MS m/z: 546 (M^{t}+1).

Preparation of Compound mMPS-2

Yield 326%, white solid.

EI-MS m/z: 898 (M^{t}).

### Example 21. Preparation of Compound mMPS-3

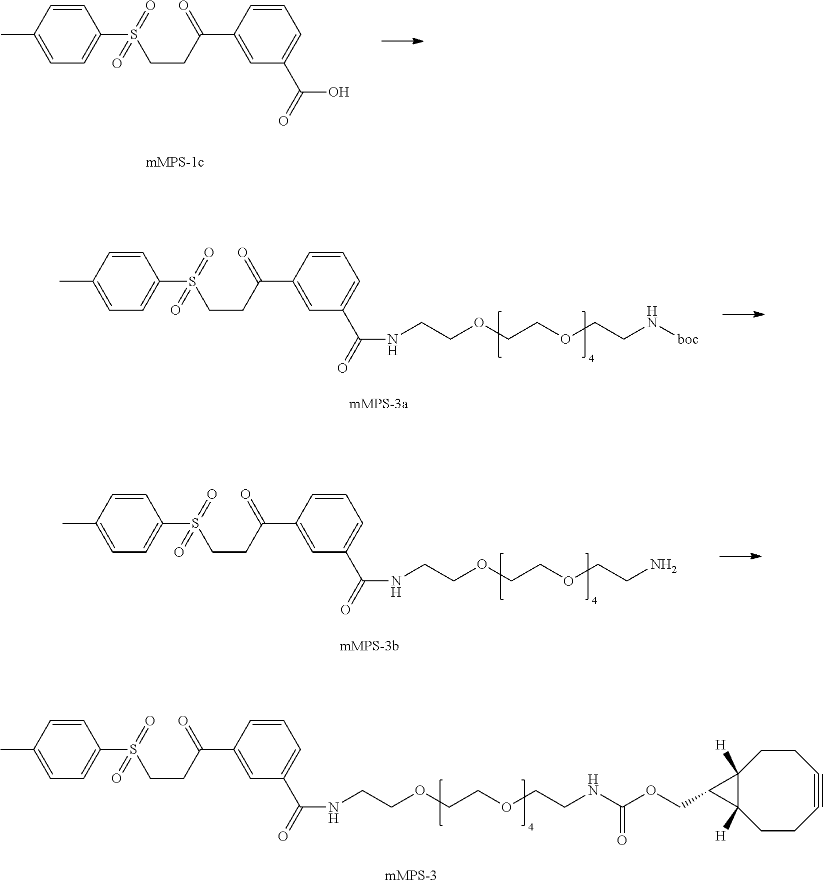

Preparation of Compound mMPS-3a

A homogeneous solution of mMPS-1c (158 mg, 0.48 mmol) and L-8 (297 mg, 0.57 mmol) in anhydrous DMF (3 mL) at room temperature under N₂ atmosphere was treated with DIPEA (0.25 mL, 1.43 mmol), HBTU (270 mg, 0.71 mmol) and stirred for 3 hrs. The reaction was quenched with water (10 mL) and extracted with EA (15 mL×2). The organic layer was washed brine (10 mL), dried over anhydrous Na₂SO₄, filtered, and concentrated in vacuo. The residue was purified by flash chromatography (DCM: MeOH=15: 1) to give the title compound mMPS-3a (377 mg, quant).

EI-MS m/z: 695 (M^{t}).

Preparation of Compound mMPS-3b

A homogeneous solution of mMPS-3a (100 mg, 0.14 mmol) in dry DCM (3 mL) at 0° C. under N₂ atmosphere was treated with 4N HCl in dioxane (360 μL, 1.44 mmol) and stirred for 2 hours. After the reaction mixture was concentrated in vacuo, mMPS-3b (91 mg, quant) was used directly in the next reaction without purification.

EI-MS m/z: 595 (M^{t}).

Preparation of Compound mMPS-3

A homogeneous solution of mMPS-3b (91 mg, 0.144 mmol) and BCN-PNP (55 mg, 0.173 mmol) in anhydrous DMF (2 mL) at room temperature under N₂ atmosphere was treated with DIPEA (75 μL, 0.433 mmol) and stirred at room temperature for 3 hours. The reaction was quenched with water (5 mL) and extracted with EA (8 mL×2). The organic layer was washed with brine (8 mL), dried over anhydrous Na₂SO₄, filtered, and concentrated in vacuo. The residue was purified by flash chromatography (DCM: MeOH=15: 1) to give the title compound mMPS-3 (44 mg, 38%).

EI-MS m/z: 771 (M^{t}).

### Example 22. Preparation of Compound mMPS-4

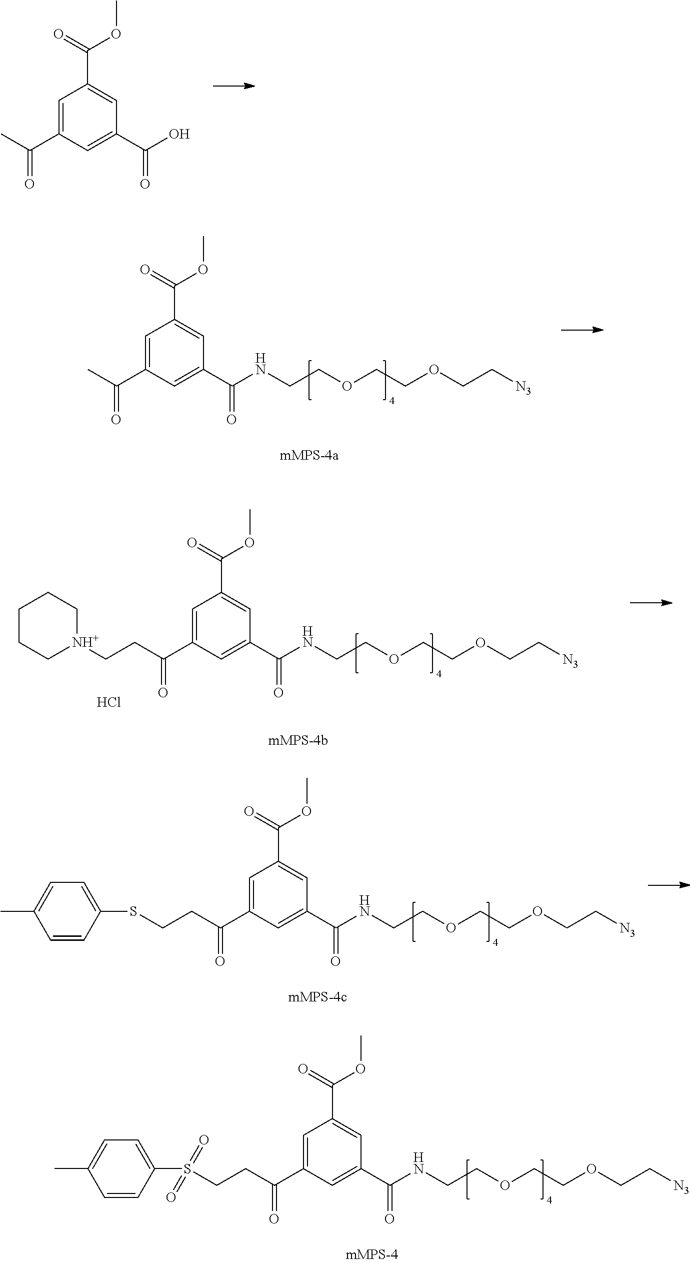

Compound mMPS-4 was synthesized via a similar manner to the preparation method of the compound mMPS-1 of Example 20.

Preparation of Compound mMPS-4a

Yield 99%.

EI-MS m/z: 511 (M^{t}).

Preparation of Compound mMPS-4b

Yield 99%.

EI-MS m/z: 608 (M^{t}).

Preparation of Compound mMPS-4c

Yield 50%.

EI-MS m/z: 647 (M^{t}).

Preparation of Compound mMPS-4

Yield 8%.

EI-MS m/z: 679 (M^{t}).

### Example 23. Preparation of Compound Mal-1 and Mal-2

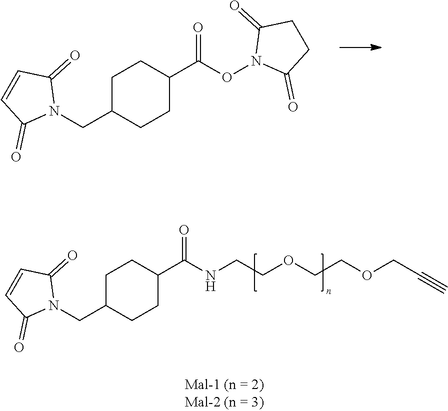

A homogeneous solution of N-succinimidyl 4-(N-maleimidomethyl)cyclohexanecarboxylate (30 mg, 0.09 mmol) and L-2 (18 mg, 0.096 mmol) in dry DCM at room temperature under N₂ atmosphere was treated with DIPEA (16 μL, 0.09 mmol) and stirred to room temperature for 45 minutes. The reaction was diluted with DCM (15 mL) and washed with 1N HCl (10 mL), brine (10 mL), dried over anhydrous Na₂SO₄, filtered, and concentrated in vacuo. The residue was purified by flash chromatography (DCM: MeOH=15: 1) and prep-HPLC to give the title compound Mal-1 (14.3 mg, 39) as a white gum.

EI-MS m/z: 407 (M⁺+1).

Compound Mal-2 was synthesized via a similar synthetic route as described above for the compound Mal-1.

Yield 61%, white gum.

EI-MS m/z: 4519 (M⁺+1).

### Example 24. Preparation of Int-TG

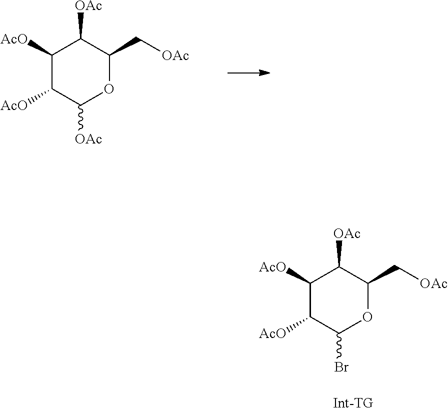 [SMILES: CC(=O)OC[C@H]1OC(OC(C)=O)[C@H](OC(C)=O)[C@@H](OC(C)=O)[C@H]1OC(C)=O ; CC(=O)OC[C@H]1OC(Br)[C@H](OC(C)=O)[C@@H](OC(C)=O)[C@H]1OC(C)=O]

β-D-galactose pentaacetate (5.0 g, 12.81 mmol) was dissolved in 33% HBr in AcOH (20 mL) at 0° C. under N₂ atmosphere. The mixture was warmed to room temperature. After stirring at room temperature for 4 hours, the mixture was concentrated under reduced pressure, and then EA (1000 mL) and saturated sodium bicarbonate (1000 mL) were added. The organic layer was dried over anhydrous Na₂SO₄, filtered, and concentrated under reduced pressure. The residue was purified by column chromatography to obtain compound Int-TG (5.2 g, 99%).

¹H NMR (400 Hz, CDCl3) δ6.70 (d, J=4.0 Hz, 1H), 5.52 (d, J=2.4 Hz, 1H), 5.41 (dd, J=7.6, 2.8 Hz, 1H), 5.05 (dd, J=6.4, 4.0 Hz, 1H), 4.49 (t, J=6.4 Hz, 1H), 4.22-4.09 (m, 2H), 2.16-2.01 (m, 12H).

### Example 25. Preparation of Compounds L-9 and L-10

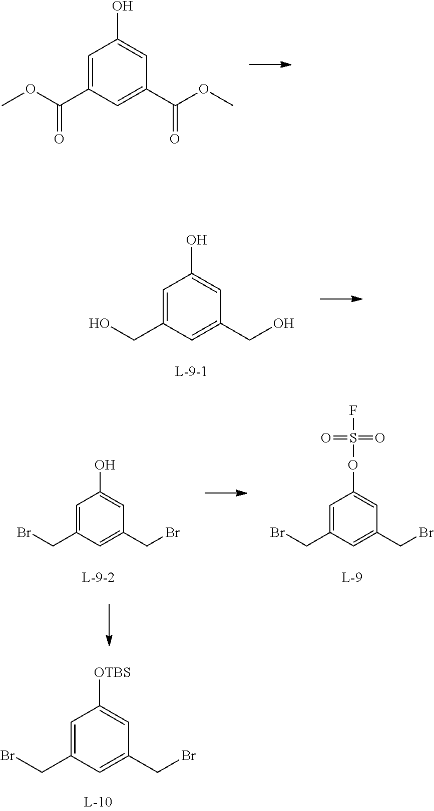

Preparation of Compound L-9-1

To a solution of dimethyl 5-hydroxyisophthalate (5 g, 23.79 mmol) in dry THF (300 mL) was slowly added LAH (3.6 g, 95.15 mmol) at −78° C. under N₂ atmosphere. The reaction mixture was stirred at room temperature for 17 hours. After the reaction was completed, 15% NaOH solution (4 mL), H₂O (8 mL) and EA (100 mL) were added and then the reaction mixture was stirred for 1 hour. The mixture was filtered, and concentrated under reduced pressure. The residue was purified by column chromatography to obtain compound L-9-1 (3.02 g, 82%).

¹H NMR (400 MHz, DMSO-d6) δ 9.21 (s, 1H), 6.66 (s, 1H), 6.58 (s, 2H), 5.07 (t, J=6.0 Hz, 2H), 4.38 (d, J=4.6 Hz, 4H).

Preparation of Compound L-9-2

Compound L-9-1 (2 g, 12.97 mmol) was dissolved in HBr (5.0 mL, 33% in AcOH) under N₂ atmosphere. After stirring at 60° C. for 18 hours, the reaction was quenched by addition of NaHCO₃ solution (pH-8). Distilled water (50 mL) and EA (100 mL×2) were added in reaction mixture. The organic layer was dried over anhydrous Na₂SO₄, filtered, and concentrated under reduced pressure. The residue was purified by column chromatography to obtain compound L-9-2 (2.9 g, 80%).

¹H NMR (400 MHz, CDCl₃) δ 6.99 (s, 1H), 6.81 (s, 2H), 4.85 (s, 1H), 4.41 (s, 2H).

### Preparation of Compound L-9

To a solution of compound L-1-2 (1.0 g, 3.57 mmol) in DCM (35 mL) was added TEA (0.45 mL, 3.21 mmol) at room temperature under N₂ atmosphere. SO₂F2 gas was introduced via balloon, and the mixture was stirred at room temperature for 1 hour. The mixture was washed with DCM (50 mL) and water (30 mL). The organic layer was washed with NaHCO₃ aqueous solution, dried over anhydrous Na₂SO₄, filtered, and concentrated under reduced pressure. The residue was purified by column chromatography to obtain compound L-2 (941.7 mg, 73%).

¹H NMR (400 Hz, CDCl3) δ7.47 (s, 1H), 7.32 (s, 2H), 4.46 (s, 4H).

Preparation of Compound L-10

To solution of compound L-9-2 (100 mg, 0.36 mmol) in dry DCM (3 mL) was added imidazole (27 mg, 0.39 mmol) and TBDMS-Cl (59 mg, 0.39 mmol) at room temperature under N₂ atmosphere. After stirring for 16 hours, distilled water (50 mL) and EA (100 mL) were added to the reaction mixture. The organic layer was dried over anhydrous Na₂SO₄, filtered, and concentrated under reduced pressure. The residue was purified by column chromatography to obtain compound L-10 (110 mg, 79%).

¹H NMR (400 MHz, CDCl₃) δ 7.00 (s, 1H), 6.80 (s, 2H), 4.41 (s, 4H), 0.99 (s, 9H), 0.21 (s, 6H).

### Example 26. Preparation of Compound L-11

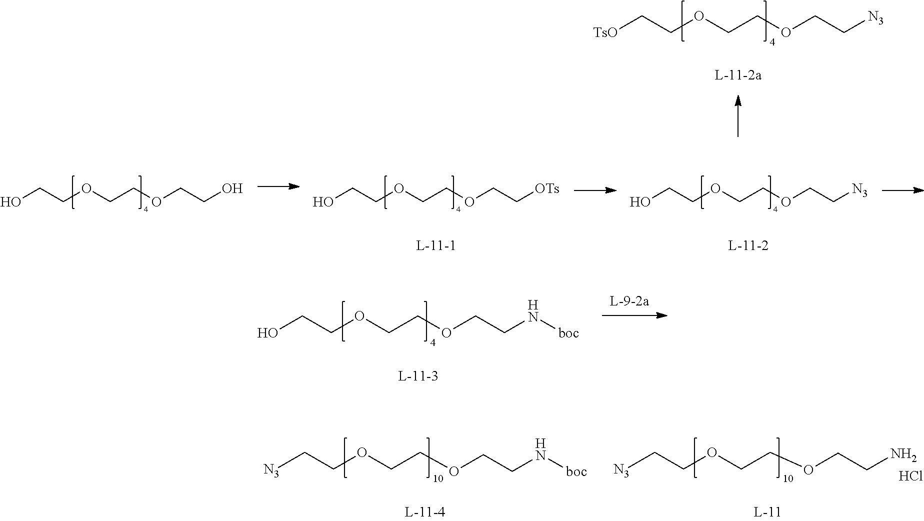

Preparation of Compound L-11-1

To a solution of hexaethylene glycol (5.0 g, 17.71 mmol) in anhydrous DCM (178 mL) was added KI (294 mg, 1.77 mmol), Ag₂₀ (4.92 g, 19.48 mmol), and p-TsC1 (3.7 g, 19.48 mmol) under N₂ atmosphere. The mixture was stirred overnight at room temperature. After the reaction was completed, the mixture was filtered through Celite®, and the Celite® plug was washed with DCM (100 mL). The filtrate was concentrated under reduced pressure. The residue was purified by column chromatography to obtain compound L-11-1 (5.98 g, 73%).

¹H NMR (400 Hz, CDCl3) δ7.80 (d, J=8.4 Hz, 2H), 7.35 (d, J=8.4 Hz, 2H), 4.16 (t, J=4.8 Hz, 2H), 3.71-3.58 (m, 22H), 2.88 (br, 1H), 2.45 (s, 3H).

### Preparation of Compound L-11-2

To a solution of compound L-11-1 (5.98 g, 13.7 mmol) in DMF (30 mL) was added NaN₃ (1.34 g, 20.55 mmol) under N₂ atmosphere. The mixture was stirred at 110° C. for 1 hour and concentrated under reduced pressure. The residue was purified by column chromatography to obtain compound L-11-2 (4.1 g, 97%).

¹H NMR (400 Hz, CDCl3) δ3.72-3.60 (m, 22H), 3.39 (t, J=4.8 Hz, 2H), 2.78 (br, 1H).

Preparation of Compound L-11-2a

Compound L-11-2 (1.9 g, 6.18 mmol) was dissolved in DCM (20 mL) under N₂ atmosphere, and triethyamine (2.0 mL, 14.22 mmol) and p-TsC1 (2.4 g, 12.36 mmol) were added thereto. The mixture was stirred overnight at room temperature. After the reaction was completed, the mixture was concentrated under reduced pressure. The residue was purified by column chromatography to obtain a compound L-11-2a (2.58 g, 91%).

¹H NMR (400 Hz, CDCl3) δ7.80 (d, J=8.4 Hz, 2H), 7.35 (d, J=8.4 Hz, 2H), 4.16 (t, J=4.8 Hz, 2H), 3.70-3.61 (m, 16H), 3.56 (s, 1H), 3.39 (t, J=4.8 Hz, 2H), 2.45 (s, 3H).

EI-MS m/z: 462 (M⁺+1).

Preparation of Compound L-11-3

To a solution of compound L-11-2 (1.0 g, 3.25 mmol) in EtOH (5 mL) was added 5% Pd/C (1.04 g, 0.49 mmol) under H₂ atmosphere. The mixture was stirred at room temperature for 4 hours. The mixture was filtered through Celite® to remove Pd/C, and concentrated under reduced pressure. The residue was dissolved in DCM (25 mL). Boc₂O (852.1 mg, 3.9 mmol) was added, and the resultant mixture was stirred at room temperature for 3 hours. The mixture was concentrated under reduced pressure. The residue was purified by column chromatography to produce compound L-11-3 (330 mg, 28%).

¹H NMR (400 Hz, CDCl3) δ5.19 (br s, 1H), 3.73 (t, J=4.8 Hz, 2H), 3.67 (s, 12H), 3.63-3.60 (m, 6H), 3.54 (t, J=5.2 Hz, 2H), 3.34-3.27 (m, 1H), 1.44 (s, 9H).

EI-MS m/z: 382 (M⁺+1).

Preparation of Compound L-11-4

A homogeneous solution of compound L-11-3 (450 mg, 1.18 mmol) in anhydrous THF (10 mL) under N₂ atmosphere at 0° C. was treated with NaH (60% dispersion in mineral oil, 47.2 mg, 1.18 mmol) After the mixture was stirred at 0° C. for 20 minutes, L-11-2a (544.5 mg, 1.18 mmol) was added thereto. The reaction was allowed to warm up to room temperature and stirred overnight. The reaction was allowed to cool, quenched with MeOH (5 mL), and concentrated under reduced pressure. The residue was purified by column chromatography to obtain compound L-11-4 (582.9 mg, 74%).

Preparation of Compound L-11

To a solution of compound L-11-4 (582.9 mg, 0.87 mmol) in DCM (3 mL) was added 4M HCl (in 1,4-dioxane, 1 mL) at 0° C. under N₂ atmosphere. The mixture was stirred at room temperature for 2 hours. The mixture was concentrated to obtain compound L-11 (527.6 mg, quant).

EI-MS m/z: 571 (M⁺+1).

### Example 27. Preparation of Compound Int-TG1 and Int-TG2

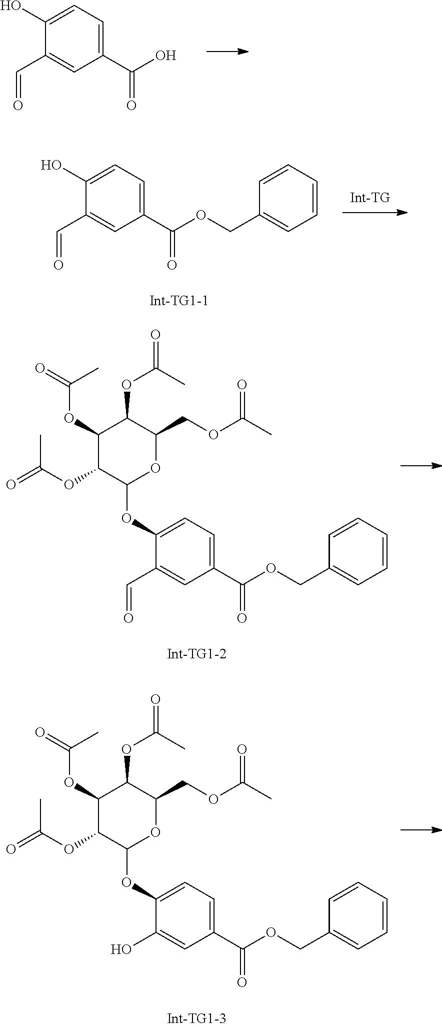

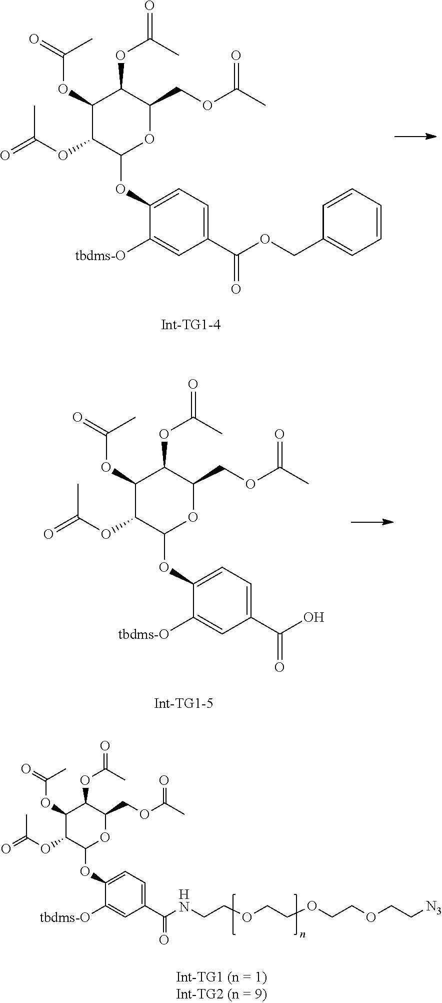

Preparation of Compound Int-TG1-1

To a solution of the 3-formyl-4-hydroxybenzoic acid (5 g, 43.06 mmol) in DMF (100 mL) was added benzyl bromide (5.1 mL, 43.06 mmol) and NaHCO₃(2.53 g, 43.06 mmol) at room temperature under N₂ atmosphere. The mixture was stirred overnight at room temperature under N₂ atmosphere. The reaction was extracted with EA (200 mL×2) and distilled water (100 mL). The obtained organic layer was dried over anhydrous Na₂SO₄, filtered, and concentrated under reduced pressure. The residue was purified by column chromatography to obtain compound Int-TG1-1 (2.56 g, 39%).

¹H NMR (400 Hz, CDCl3) δ11.41 (s, 1H), 9.95 (s, 1H), 8.34 (d, J=2.0 Hz, 1H), 8.23 (dd, J=6.4 Hz, 2.4 Hz, 1H), 7.46-7.35 (m, 5H), 7.04 (d, J=9.2 Hz, 1H), 5.37 (s, 2H).

### Preparation of Compound Int-TG1-2

To a solution of compound Int-TG1-1 (1.0 g, 3.90 mmol) and compound Int-TG (1.6 g, 3.90 mmol) in anhydrous MeCN (30 mL) was added molecular sieves (8 g) and Ag₂O (3.62 g, 15.61 mmol) at room temperature under N₂ atmosphere. The mixture was stirred at room temperature for 1 hour, then filtered through Celite®. The organic layer was dried over Na₂SO₄, filtered, and concentrated under reduced pressure. The residue was purified by column chromatography to obtain compound Int-TG1-2 (2.1 g, 92%).

¹H NMR (400 Hz, CDCl3) δ10.34 (s, 1H), 8.55 (d, J=2.0 Hz, 1H), 8.26 (dd, J=6.8, 2.0 Hz, 1H), 7.45-7.35 (m, 5H), 7.17 (d, J=8.8 Hz, 1H), 5.63-5.60 (m, 1H), 5.50 (d, J=3.6 Hz, 1H), 5.37 (s, 2H), 5.23 (d, J=8.0 Hz, 1H), 5.16 (dd, J=7.2, 3.6 Hz, 1H) 4.24-4.10 (m, 4H), 2.20 (s, 3H), 2.10-2.03 (m, 9H).

Preparation of Compound Int-TG1-3

To a solution of compound Int-TG1-2 (2.1 g, 3.58 mmol) in DCM (30 mL) was added m-CPBA (2.65 g, 10.74 mmol) at 0° C. under N₂ atmosphere. After stirring for 7 hours at 0° C., the mixture was quenched by addition of saturated sodium bicarbonate (40 mL×2). The mixture was separated and the organic layers were washed with brine, dried over Na₂SO₄, filtered, and concentrated under reduced pressure. The residue was dissolved in DCM (5 mL), and hydrazine-hydrate (261 μL, 5.37 mmol) was added to the mixture at 0° C. under N₂ atmosphere. After stirring at 0° C. for 1 hour, EA (30 mL×2) and 1M HCl aqueous solution (10 mL) were added. The obtained organic layer was dried over anhydrous Na₂SO₄, filtered, and concentrated under reduced pressure to obtain compound Int-TG1-3 (1.1 g, 55%).

EI-MS m/z: 574 (M⁺+Na)

Preparation of Compound Int-TG1-4

To a solution of compound Int-TG1-3 (280 mg, 0.49 mmol) in DCM (5 mL) was added TBDMS-OTf (224 μL, 0.97 mmol) and Et3N (207 μL, 1.46 mmol) at 0° C. under N₂ atmosphere. The mixture was stirred for 1.5 hours at room temperature, and then quenched by addition of citric acid (20 ml). The organic layer was washed with brine (20 mL), dried over Na₂SO₄, filtered, and concentrated under reduced pressure. The residue was purified by column chromatography to obtain compound Int-TG1-4 (246.3 mg, 68%).

¹H NMR (400 Hz, CDCl3) δ7.67 (d, J=8.4 Hz, 1H), 7.57 (s, 1H), 7.44-7.34 (m, 5H), 7.02 (d, J=8.4 Hz, 1H), 5.49-5.44 (m, 2H), 5.30 (s, 2H), 5.19 (d, J=7.6 Hz, 1H), 5.10 (dd, J=6.8, 3.2 Hz, 1H) 4.20-4.11 (m, 2H), 4.05 (t, J=6.8 Hz, 2H), 2.19 (s, 3H), 2.04 (s, 3H), 2.01 (d, J=6.0 Hz, 6H), 1.02 (s, 9H), 0.20 (d, J=15.6 Hz, 6H).

Preparation of Compound Int-TG1-5

To a solution of compound Int-TG1-4 (283.2 mg, 0.41 mmol) in EA (5 mL) was added Pd/C (5%, 87.5 mg, 0.04 mmol) at room temperature under Hz. The mixture was stirred for 1 hour and filtered through Celite®, and then concentrated under reduced pressure. The compound Int-TG1-5 was used directly in the next step without further purification (246 mg, quant).

¹H NMR (400 Hz, CDCl3) δ7.67 (d, J=8.8 Hz, 1H), 7.57 (s, 1H), 7.05 (d, J=8.4 Hz, 1H), 5.49-5.45 (m, 2H), 5.22 (d, J=7.6 Hz, 1H), 5.12 (dd, J=7.2, 3.6 Hz, 1H) 4.20-4.06 (m, 4H), 2.19 (s, 3H), 2.05 (s, 3H), 2.02 (d, J=7.6 Hz, 6H), 1.01 (s, 9H), 0.21 (d, J=15.2 Hz, 6H).

Preparation of Compound Int-TG1

To a solution of compound Int-TG1-5 (243.2 mg, 0.41 mmol) and 11-azido-3,6,9-trioxaundecan-1-amine (Aldrich, CAS 134179-38-7, 89.5 mg, 0.41 mmol) in DMF (5 mL) were added PyBOP (275 mg, 0.53 mmol) and DIPEA (176 μL, 1.02 mmol) at room temperature under N₂ atmosphere. The mixture was stirred for 2 hours at room temperature under N₂ atmosphere. The reaction was extracted with EA (30 mL×2) and distilled water (10 mL). The obtained organic layer was dried over anhydrous Na₂SO₄, filtered, and concentrated under reduced pressure. The residue was purified by column chromatography to obtain compound Int-TG1 (272.8 mg, 84%).

¹H NMR (400 Hz, CDCl3) δ7.34 (s, 1H), 7.31 (d, J=9.2 Hz, 1H), 7.02 (d, J=8.0 Hz, 1H), 6.73 (s, 1H), 5.48-5.44 (m, 2H), 5.19 (d, J=7.6 Hz, 1H), 5.10 (dd, J=6.4, 3.6 Hz, 1H), 4.20-4.10 (m, 2H), 4.06 (t, J=6.4 Hz, 2H), 3.66 (s, 14H), 3.38 (t, J=4.4 Hz, 2H), 2.19 (s, 3H), 2.02 (t, J=8.4 Hz, 9H), 1.00 (s, 9H), 0.20 (d, J=14.4 Hz, 6H).

EI-MS m/z: 799 (M⁺+1).

Preparation of Compound Int-TG2

To a solution of compound Int-TG1-5 (246 mg, 0.41 mmol) and L-9 (249.5 mg, 0.41 mmol) in DMF (3 mL) were added PyBOP (278 mg, 0.53 mmol) and DIPEA (179 μL, 1.02 mmol) at room temperature under N₂ atmosphere. After the mixture was stirred for 2 hours, the reaction mixture was subjected to Prep-HPLC to obtain a compound Int-TG2 (384.6 mg, 81%). EI-MS m/z: 1152 (M⁺+1).

### Example 28. Preparation of Compound Int-TG3

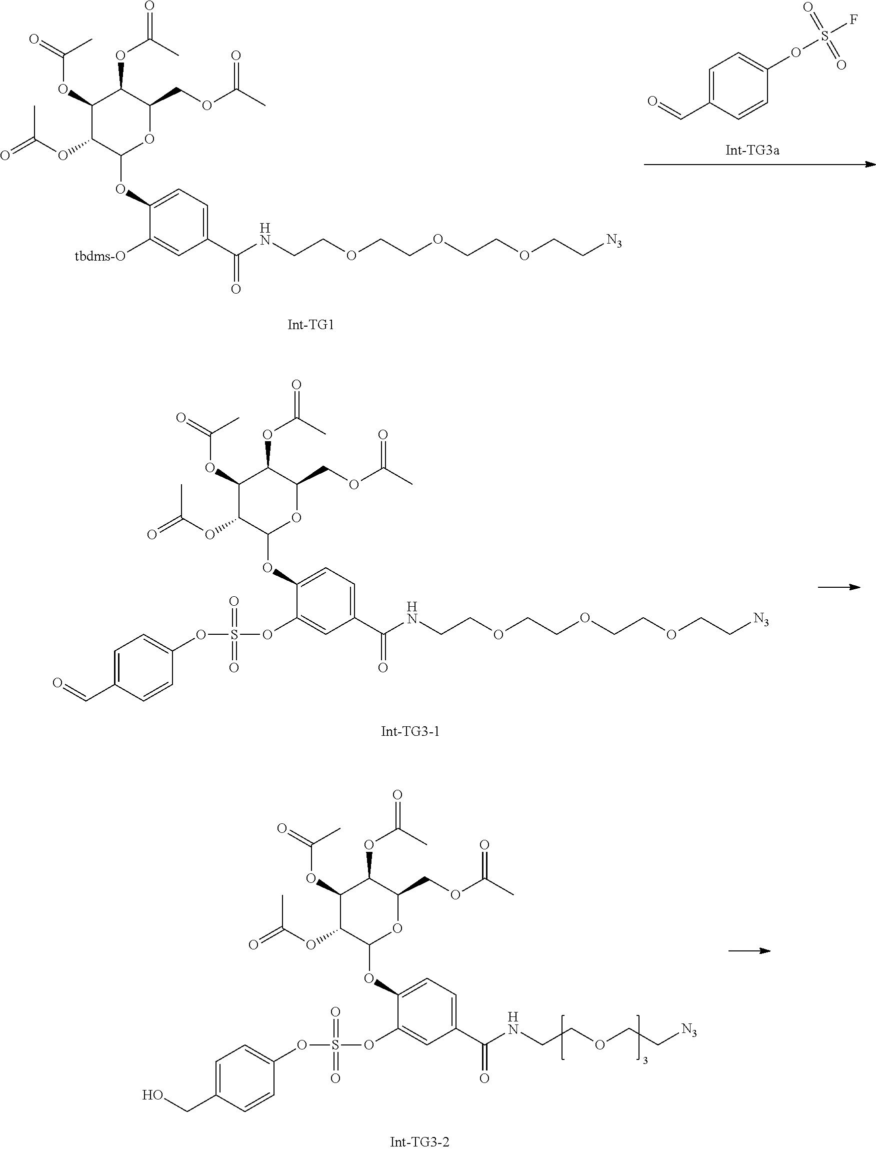

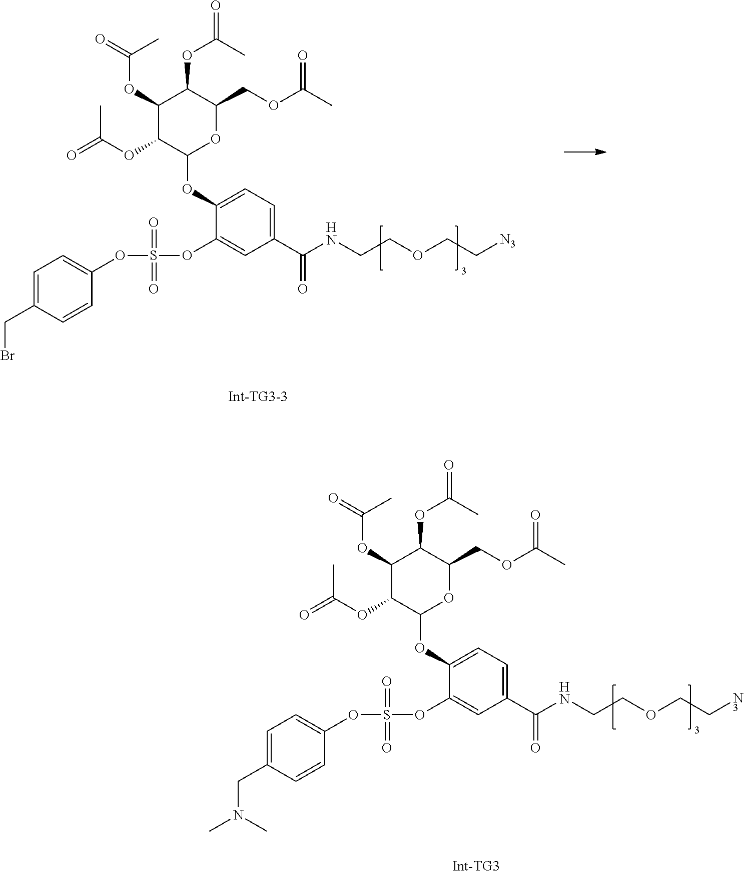

Preparation of Compound Int-TG3a

To a solution of 4-hydroxybenzaldehyde (1 g, 8.19 mmol) in DCM (3 mL) was added Et₃N (2.28 mL, 16.38 mmol) at room temperature under N₂ atmosphere. SO₂F2 gas was introduced via balloon, and the mixture was stirred at room temperature for 2 hours. The mixture was washed with DCM (30 mL×3) and brine (30 mL), the organic layer was dried over anhydrous Na₂SO₄, filtered, and concentrated under reduced pressure. The residue was purified by column chromatography to obtain compound Int-TG3a (790 mg, 63%).

¹H NMR (400 Hz, CDCl3) δ10.06 (s, 1H), 8.05 (d, J=8.0 Hz, 2H), 7.55 (d, J=8.8 Hz, 2H).

### Preparation of Compound Int-TG3-1

To a solution of compound Int-TG1 (100 mg, 0.13 mmol) and compound Int-TG3a (26 mg, 0.13 mmol) in anhydrous MeCN (3 mL) was added DBU (4 μL, 25 μmol). The mixture was stirred at room temperature for 1 hour and was washed with distilled water (10 mL) and EA (15 mL×2). The organic layer was dried over anhydrous Na₂SO₄, filtered, and concentrated under reduced pressure. The residue was purified by column chromatography to obtain compound Int-TG3-1 (103 mg, 94%).

EI-MS m/z: 869 (M⁺+1).

Preparation of Compound Int-TG3-2

To a solution of compound Int-TG3-1 (103 mg, 0.12 mmol) in THF (8 mL) was added NaBH₄ (9 mg, 0.24 mmol) at 0° C. under N₂ atmosphere. After stirring at room temperature for 2 hours, distilled water (10 mL) and EA (10 mL×2) were added. The organic layer was dried over anhydrous Na₂SO₄, filtered, and concentrated under reduced pressure to obtain compound Int-TG3-2 (101 mg, 98%).

EI-MS m/z: 871 (M⁺+1).

Preparation of Compound Int-TG3-3

To a solution of compound Int-TG3-2 (320.5 mg, 0.0.37 mmol) in DCM (3 ml) was added 1M PBr₃ in DCM (165 μL, 0.19 mmol) at 0° C. under N₂ atmosphere. After stirring at 0° C. for 2 hours. The mixture was quenched by addition of saturated sodium bicarbonate (8 mL×2). The mixture was separated and the organic layers were washed with brine, dried over Na₂SO₄, filtered, and concentrated under reduced pressure. The residue was purified by column chromatography to produce compound Int-TG3-3 (202.6 mg, 59%)

EI-MS m/z: 934 (M⁺+1).

Preparation of Compound Int-TG3

To a solution of compound Int-TG3-3 (10 mg, 0.01 mmol) in DMF (1 mL) was added dimethylamine (0.1 mL) at room temperature under N₂ atmosphere. After stirring for 10 minutes at room temperature, the reaction mixture was purified by prep-HPLC to obtain compound Int-TG3 (6 mg, 63%). EI-MS m/z: 898(M⁺+1).

### Example 29. Preparation of Compound L-12

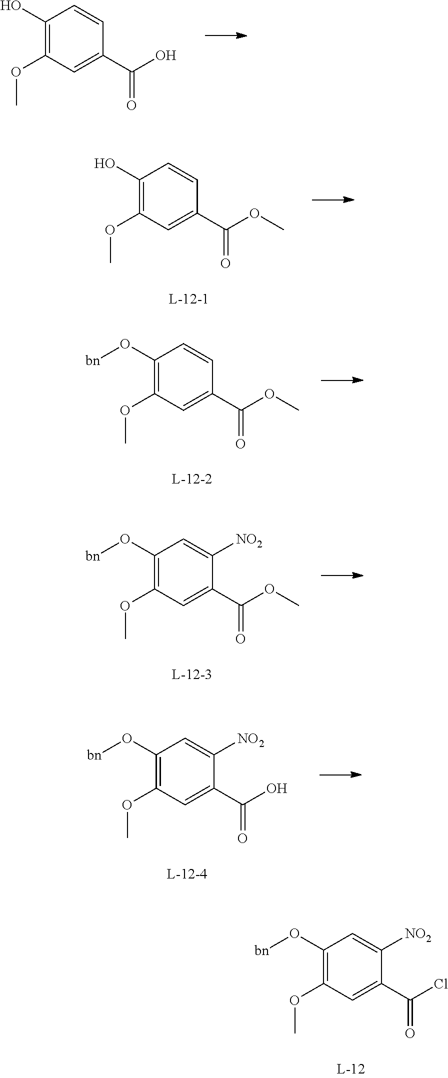

Preparation of Compound L-12-1

To a solution of vanillic acid (50.0 g, 0.30 mol) in MeOH (700 mL) was added dropwise SOCl₂ (207 mL, 2.85 mol) and the resulting mixture was stirred at 0° C. under N₂ atmosphere, and then stirred overnight at room temperature. After the reaction was completed, the mixture was concentrated under reduced pressure. The reaction was adjusted to pH 7 to 8 with saturated aqueous NaHCO₃ solution and then diluted with distilled water (100 mL) and EA (200 mL×2). The organic layer was dried over anhydrous Na₂SO₄, filtered, and concentrated under reduced pressure. The residue was purified by column chromatography to obtain compound L-12-1 (54.2 g, quant)

¹H NMR (400 MHz, CDCl₃) δ 7.64 (dd, J=6.4, 1.6 Hz, 1H), 7.55 (s, 1H), 6.94 (d, J=8.4 Hz, 1H), 6.05 (s, 1H), 3.95 (s, 3H), 3.89 (s, 3H).

### Preparation of Compound L-12-2

To a solution of compound L-12-1 (54.2 g, 0.30 mol) in DMF (200 mL) was added K₂CO₃ (61.6 g, 0.45 mol) and benzyl bromide (39.0 mL, 0.33 mol) under N₂ atmosphere. After stirring for 6 hours at 100° C., the mixture was cooled to room temperature and diluted with distilled water (100 mL) and EA (200 mL×2). The organic layer was dried over anhydrous Na₂SO₄, filtered, and concentrated under reduced pressure. The residue was purified by column chromatography to obtain compound L-12-2 (79.8 g, 98%).

¹H NMR (400 MHz, CDCl₃) δ 7.60 (dd, J=6.4, 2.0 Hz, 1H), 7.56 (d, J=2.0 Hz, 1H), 7.44-7.31 (m, 5H), 6.89 (d, J=8.4 Hz, 1H), 5.22 (s, 2H), 3.94 (s, 3H), 3.88 (s, 3H).

### Preparation of Compound L-12-3

Compound L-12-2 (79.8 g, 0.29 mol) was dissolved in acetic anhydride (550 mL) under N₂ atmosphere and then cooled to 0° C. Copper (II) nitrate hemi-(pentahydrate) (75.0 g, 0.32 mol) was added portion-wise. After stirring at 0° C. for 6 hours, the reaction was quenched with ice water (800 mL). The solid was filtered and washed with distilled water (100 mL) and hexane (200 mL×2) to obtain compound L-12-3 (85.5 g, 92%).

¹H NMR (400 MHz, CDCl₃) δ 7.52 (s, 1H), 7.45-7.35 (m, 5H), 7.08 (s, 1H), 5.22 (s, 2H), 3.98 (s, 3H), 3.91 (s, 3H).

Preparation of Compound L-12-4

To a solution of compound L-12-3 (85.5 g, 0.27 mol) in THF (800 mL) and MeOH (300 mL) was added 2N NaOH (404 mL, 0.81 mol). After stirring for 5 hours at 65° C., the reaction was cooled to room temperature and adjusted to pH 2 by addition of 2N HCl solution, and then extracted with distilled water (100 mL) and EA (300 mL×2). The organic layer was dried over anhydrous Na₂SO₄, filtered, and concentrated under reduced pressure. The residue solid was collected and washed with hexane to obtain compound L-12-4 (79.2 g, 97%).

¹H NMR (400 MHz, DMSO-d6) δ 7.69 (s, 1H), 7.47-7.35 (m, 5H), 7.03 (s, 1H), 5.24 (s, 2H), 3.91 (s, 3H).

### Preparation of Compound L-12

To a solution of compound L-12-4 (100 mg, 0.33 mmol) in anhydrous THF (500 μL) and anhydrous DCM (1.5 mL) were slowly added dropwise oxalyl chloride (42.4 μL) and 1 drop of DMF at 0° C. under N₂ atmosphere. After stirring for 30 min, the reaction mixture was concentrated under reduced pressure. Compound L-12 was used directly in the next step without further purification.

### Example 30. Preparation of Compound Mono-1

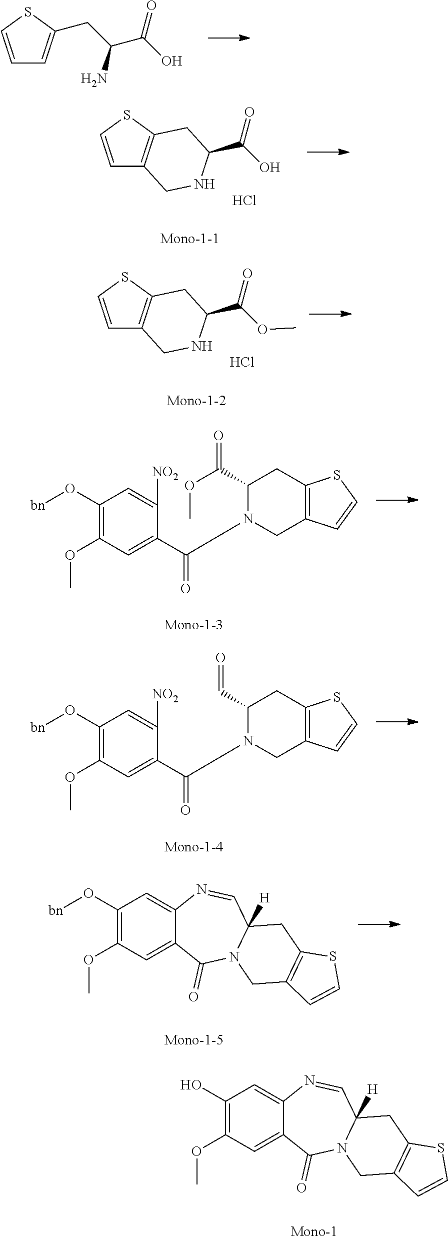

Preparation of Compound Mono-1-1

To a solution of L-2-thienylalanine (500 mg, 2.92 mmol) in distilled water (5.0 mL) was added dropwise conc. HCl (206 μL) and stirred at 0° C. under N₂ atmosphere, and then formaldehyde (37%, 261 μL, 3.5 mmol) was added thereto. The mixture was refluxed overnight. After the reaction was completed, the mixture was concentrated under reduced pressure. The residue was suspended in IPA (3.0 mL) and 4M HCl (in 1,4-dixoane, 1.0 mL) was added thereto. After stirring for 2 hours, the solid was filtered and washed with IPA (5 mL), ether (20 mL) to obtain compound Mono-1-1 (495.7 mg, 77%)

¹H NMR (400 MHz, DMSO-d6) δ 9.95 (br s, 1H), 7.48 (d, J=5.2 Hz, 1H), 6.94 (d, J=5.2 Hz, 1H), 4.48-4.44 (m, 1H), 4.28 (d, J=15.6 Hz, 1H), 4.18 (d, J=16.0 Hz, 1H), 3.39 (dd, J=11.6, 5.2 Hz, 1H), 3.17-3.10 (m, 1H). EI-MS m/z: 184 (M⁺+1).

Preparation of Compound Mono-1-2

Compound Mono-1-1 (495.7 mg, 2.25 mmol) was dissolved in MeOH (10.0 mL) under N₂ atmosphere and then cooled to 0° C. SOCl₂ (491.3 μL, 6.76 mmol) was dropwise at 0° C. And then the reaction mixture was refluxed for 3 hours. After the reaction was completed, the mixture was concentrated under reduced pressure. The residue was wash with diethyl ether (5 mL×2) to obtain compound Mono-1-2 (521.5 mg, 99%)

¹H NMR (400 MHz, DMSO-d6) δ 10.22 (br s, 2H), 7.49 (d, J=5.2 Hz, 1H), 6.94 (d, J=5.2 Hz, 1H), 4.65-4.61 (m, 1H), 4.30 (d, J=15.6 Hz, 1H), 4.19 (d, J=15.6 Hz, 1H), 3.80 (s, 3H), 3.60 (dd, J=11.6, 5.2 Hz, 1H), 3.21-3.14, (m, 1H). ELMS m/z: 198 (M⁺+1).

Preparation of Compound Mono-1-3

To a solution of compound L-12 (856.5 mg, 2.66 mmol) in anhydrous THF (3.0 ml) was added compound Mono-1-2 (518.5 mg, 2.22 mmol) dissolved in DMF (3.0 mL) and DIPEA (772.8 μL, 4.44 mmol) at 0° C., and the resulting reaction mixture was stirred at room temperature overnight. After the reaction was completed distilled water (20 mL) and EA (50 mL×2) were added to the reaction mixture. The organic layer was dried over anhydrous Na₂SO₄, filtered, and concentrated under reduced pressure. The residue was purified by column chromatography to obtain compound Mono-1-3 (888.5 mg, 89%)

ELMS m/z: 483 (M⁺+1).

Preparation of Compound Mono-1-4

To a solution of Mono-1-3 (880 mg, 1.82 mmol) in anhydrous DCM (5.0 mL) and toluene (15.0 mL) was added DIBAL (3.6 mL, 3.6 mmol, 1.0M in toluene) dropwise at −78° C. under N₂ atmosphere. The reaction mixture was stirred at −78° C. for 3 hours. The reaction was quenched with MeOH (5 mL) and 2N HCl (20.0 mL) at −78° C. Distilled water (20 mL) and EA (50 mL×2) were added to the reaction mixture. The organic layer was dried over anhydrous Na₂SO₄, filtered, and concentrated under reduced pressure. The residue was purified by column chromatography to obtain compound Mono-1-4 (701.9 mg, 85%).

EI-MS m/z: 453(M⁺+1).

Preparation of Compound Mono-1-5

To a solution of Mono-1-4 (700 mg, 1.55 mmol) in THF (15.0 mL) and distilled water (3.0 mL) was added Na₂S₂O₄ (2.2 g, 12.4 mmol) at room temperature for 4 hours. After the reaction was completed, the reaction was quenched with MeOH (5 mL). The reaction mixture was concentrated under reduced pressure. The residue was suspended in toluene (20 mL) and evaporated to help remove any remaining water. The obtained white solid was completely dried by leaving on a high vacuum overnight. The residue was suspended in anhydrous MeOH (10 mL) followed by addition of acetyl chloride (1.1 mL, 15.5 mmol). After 15 minutes the cloudy solution was filtered and the solid wash with anhydrous MeOH (5 mL×2). The filtrate was stirred for 2 hours. The reaction mixture was quenched with NaHCO₃ solution (pH-7), then distilled water (20 mL) and EA (50 mL×2) were added to the reaction mixture. The organic layer was dried over anhydrous Na₂SO₄, filtered, and concentrated under reduced pressure. The residue was purified by column chromatography to obtain compound Mono-1-5 (701.9 mg, 85%)

¹H NMR (400 MHz, CDCl₃) δ 7.55 (d, J=5.6 Hz, 1H), 7.47 (m, 5H), 7.22 (d, J=5.2 Hz, 1H), 6.95 (d, J=5.2 Hz, 1H), 6.85 (s, 1H), 5.26-5.14 (m, 2H), 4.98 (d, J=16.4 Hz, 1H), 4.44 (d, J=16.8 Hz, 1H), 4.08-4.02 (m, 1H), 3.98 (s, 3H), 3.32-3.26 (m, 1H).

EI-MS m/z: 453 (M⁺+1).

Preparation of Compound Mono-1

To a solution of Mono-1-5 (60 mg, 0.15 mmol) in anhydrous DCM (3 mL) at 0° C. was added methanesulfonic acid (700 μL) in DCM (2.0 mL), and the resulting mixture was stirred for 2 hours at ° C. The reaction was quenched with NaHCO₃ solution (pH —7), then distilled water (5 mL) and EA (20 mL×2) were added to the reaction mixture. The organic layer was dried over anhydrous Na₂SO₄, filtered, and concentrated under reduced pressure. The residue was purified by column chromatography to obtain compound Mono-1 (38.3 mg, 82%).

¹H NMR (400 MHz, CDCl₃) δ 7.58 (d, J=5.6 Hz, 1H), 7.54 (s, 1H), 7.23 (d, J=5.2 Hz, 1H), 6.95 (d, J=5.2 Hz, 1H), 6.89 (s, 1H), 6.06 (s, 1H), 5.30 (s, 1H), 4.99 (d, J=16.4 Hz, 1H), 4.44 (d, J=16.4 Hz, 1H), 4.10-4.04 (m, 1H), 3.99 (s, 3H), 3.32-3.26 (m, 1H).

EI-MS m/z: 315(M⁺+1).

### Example 31. Preparation of Compound Mono-2

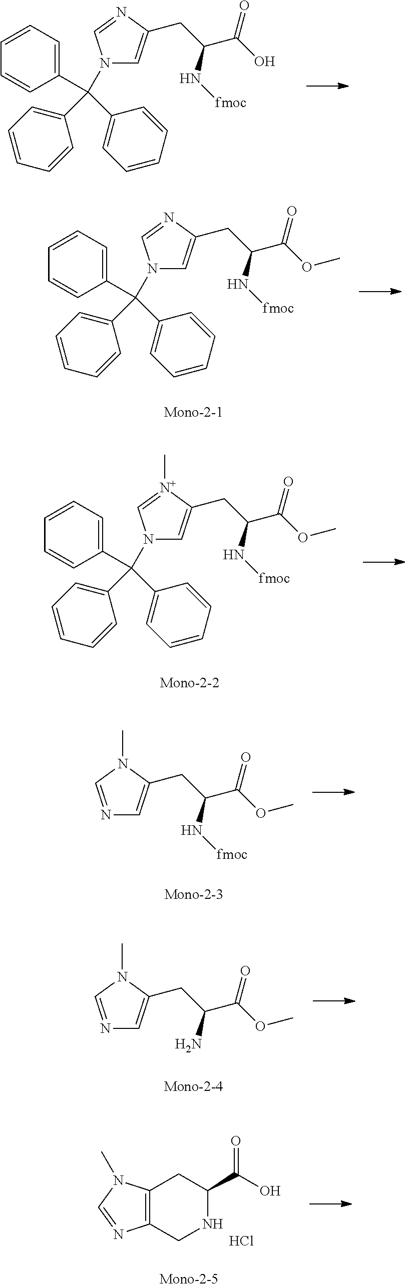

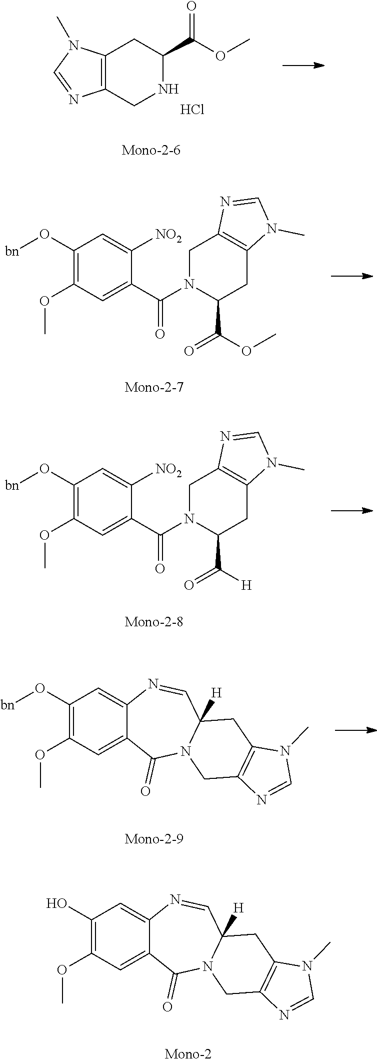

Preparation of Compound Mono-2-1

To a solution of Fmoc- His(Trt)—OH (15.0 g, 24.2 mmol), and HOBT (5.0 g, 24.2 mmol) in anhydrous THF (200 mL) was added DCC (1.15 g, 8 mmol) in THF (40 mL) and MeOH (20 mL) dropwise over 30 minutes at −13° C. The reaction mixture is allowed to warm slowly to room temperature while stirring for 5 hours. After the reaction was completed, distilled water (50 mL) and DCM (200 mL×2) were added to the reaction mixture. The organic layer was dried over anhydrous Na₂SO₄, filtered, and concentrated under reduced pressure. The residue was purified by column chromatography to obtain compound Mono-2-1 (13.0 g, 84%)

¹H NMR (400 MHz, CDCl₃) δ 7.75 (d, J=7.6 Hz, 2H), 7.62 (t, J=7.6 Hz, 2H), 7.41-7.28 (m, 14H), 7.15-7.06 (m, 7H), 6.54 (s, 1H), 6.52 (d, J=7.6 Hz, 1H), 4.66-4.59 (m, 1H), 4.38-4.22 (m, 2H), 3.63 (s, 3H), 3.07 (t, J=6.4 Hz, 1H). EI-MS m/z: 634 (M⁺+1).

Preparation of Compound Mono-2-2

To a solution of compound Mono-2-1 (13 g, 20.51 mmol) in DMF (50 mL) was added methyl iodide (3.8 mL, 61.54 mmol) at 0° C. The reaction mixture was allowed to warm to room temperature and stirred for 5 hours. After the reaction was completed, the mixture was concentrated under reduced pressure. The residue was purified by column chromatography to obtain compound Mono-2-2 (11 g, 83%)

¹H NMR (400 MHz, CDCl₃) δ 8.42 (s, 1H), 7.76 (d, J=7.6 Hz, 2H), 7.70-7.60 (m, 2H), 7.46-7.20 (m, 19h), 6.89 (s, 1H), 6.60 (d, J=7.2 Hz, 1H), 4.70-4.62 (m, 1H), 4.30-4.12 (m, 3H), 4.01 (s, 3H), 3.67 (s, 3H), 3.50-3.28 (m, 2H). ELMS m/z: 648 (M⁺+1).

Preparation of Compound Mono-2-3

To a solution of compound Mono-2-2 (11 g, 16.95 mmol) in DCM (150 mL) was added TFA (40 mL) and triethylsilane (8.12 mL, 50.86 mmol) under N₂ atmosphere at 0° C. The reaction was allowed to warm to room temperature and stirred for 6 hours. After the reaction was completed, the mixture was concentrated under reduced pressure. The residue was purified by column chromatography to obtain compound Mono-2-3 (6.25 g, 91%).

¹H NMR (400 MHz, CDCl₃) δ 8.87 (s, 1H), 7.78 (d, J=7.2 Hz, 2H), 7.59 (d, J=7.6 Hz, 2H), 7.45-7.30 (m, 4H), 7.09 (s, 1H), 5.69 (d, J=6.0 Hz, 1H), 4.64-4.50 (m, 2H), 4.48-4.38 (m, 1H), 3.79 (s, 6H), 3.51-3.44 (m, 1H), 3.29-3.10 (m, 2H). EI-MS m/z: 407 (M⁺+1).

Preparation of Compound Mono-2-4

To a solution of compound Mono-2-3 (6.25 g, 15.37 mmol) in DCM (150 mL) was added piperidine (3.0 mL, 30.74 mmol) under N₂ atmosphere at 0° C. The reaction mixture was allowed to warm to room temperature and stirred for 7 hours. After the reaction was completed, the mixture was concentrated under reduced pressure to obtain compound Mono-2-4 (2.65 g, 95%).

¹H NMR (400 MHz, CDCl₃) δ 7.51 (s, 1H), 6.87 (s, 1H), 3.78-3.69 (m, 4H), 3.63 (s, 3H), 3.09-2.84 (m, 2H). EI-MS m/z: 184 (M⁺+1).

Preparation of Compound Mono-2-5

Compound Mono-2-4 (2.65 g, 14.46 mmol) was dissolved in distilled water (100 mL) under N₂ atmosphere and then the reaction mixture was cooled to 0° C. Conc-HCl (2.5 mL) was added dropwise at 0° C., followed by formaldehyde (37%, 2.2 mL, 28.93 mmol). The reaction mixture was refluxed overnight. After the reaction was completed, the mixture was concentrated under reduced pressure. The residue was suspended in IPA (20 mL) and 4M HCl (in 1,4-dioxane, 4.0 mL) was added. The reaction mixture was stirred for 2 hours. The solid was filtered and washed with IPA (5 mL) and ether (10 mL×2) to obtain compound Mono-2-5 (3.14 g, 99%)

EI-MS m/z: 182 (M⁺+1).

Preparation of Compound Mono-2-6

To a solution of Mono-2-5 (3.14 g, 14.43 mmol) in MeOH (100 mL) was added dropwise SOCl₂ (2.5 mL, 35.15 mmol) at 0° C. After the reaction mixture was refluxed for 5 hours, the mixture was concentrated under reduced pressure. The residue was washed with ether (25 mL×2) to obtain compound Mono-2-6 (2.18g, 65%).

EI-MS m/z: 196 (M⁺+1).

Preparation of Compound Mono-2-7

To a solution of compound L-12 (3.93 g, 12.23 mmol) and compound Mono-2-6 (2.18 g, 9.41 mmol) in anhydrous THF (30 ml) and DMF (30 mL) was added DIPEA (4.9 mL, 28.22 mmol) at 0° C. After stirring for 2 hours at room temperature, the mixture was quenched with distilled water (200 mL) and EA (1000 mL). The organic layer was dried over anhydrous Na₂SO₄, filtered, and concentrated under reduced pressure. The residue was purified by column chromatography to obtain compound Mono-2-7 (2.81 g, 62%).

EI-MS m/z: 481 (M⁺+1).

Preparation of Compound Mono-2-8

To a solution of compound Mono-2-7 (2.5 g, 5.20 mmol) in anhydrous DCM (12.5 mL) and toluene (37.5 mL) was added DIBAL (10.4 mL, 10.41 mmol, 1.0M in toluene) dropwise at −78° C. under N₂ atmosphere. After stirring for 5 hours at −78° C., the mixture was quenched with MeOH (1.0 mL) and 2N HCl (100 mL) at the same temperature. The mixture was diluted in succession with water (100 mL) and DCM (200 mL), and then the organic layer was dried over anhydrous Na₂SO₄, filtered, and concentrated under reduced pressure. The residue was purified by column chromatography to obtain compound Mono-2-8 (1.21 g, 52%).

EI-MS m/z: 451 (M⁺+1).

Preparation of Compound Mono-2-9

To a solution of compound Mono-2-8 (1.2 g, 2.66 mmol) in THF (100 mL) and distilled water (70 mL) was added Na₂S₂O₄ (3.7 g, 21.31 mmol) at room temperature. After stirring for 6 hours, the reaction was quenched with MeOH (20 mL). The mixture was concentrated under reduced pressure three times by using toluene as a co-solvent, thereby removing water. The obtained yellow solid was suspended in anhydrous MeOH (200 mL), and acetyl chloride (1.9 mL, 26.64 mmol) was added thereto. After stirring for 15 minutes, the reaction mixture was adjusted to have pH 8 by addition of saturated NaHCO₃ solution and diluted with distilled water (250 mL), MeOH (250 mL) and DCM (200 mL). The organic layer was dried over anhydrous Na₂SO₄, filtered, and concentrated under reduced pressure. The residue was purified by column chromatography to obtain compound Mono-2-9 (918 mg, 78%).

¹H NMR (400 MHz, CDCl₃) δ 7.58 (d, J=5.6 Hz, 1H), 7.57 (s, 1H), 7.52 (s, 1H), 7.49-7.27 (m, 5H), 6.84 (s, 1H), 5.26-5.15 (m, 2H), 4.66 (s, 2H), 4.16 (t, J=6.0 Hz, 1H), 3.97 (s, 3H), 3.67 (s, 3H), 3.22-2.94 (m, 2H). EI-MS m/z: 403 (M⁺+1).

Preparation of Compound Mono-2

To a solution of compound Mono-2-9 (50 mg, 0.12 mmol) in anhydrous DCM (2 mL) was added methanesulfonic acid (0.1 mL) in DCM (0.2 mL) at 0° C. After stirring for 1 hour at room temperature, the mixture was adjusted to have pH 8 by addition of saturated NaHCO₃ solution. The residue was purified by Prep-HPLC to obtain compound Mono-2 (27 mg, 71%)

¹H NMR (400 MHz,CD₃OD) δ8.34 (br s, 1H), 7.68 (s, 1H), 7.28 (s, 1H), 6.42 (s, 1H), 4.77 (d, J=16.0 Hz, 1H), 4.56 (d, J=16.0 Hz, 1H), 4.33 (d, J=7.6 Hz, 1H), 4.10-4.02 (m, 1H), 3.84 (s, 3H), 3.66 (s, 3H), 3.02-2.82 (m, 2H). ELMS m/z: 313 (M⁺+1).

### Example 32. Preparation of Compound Mono-3

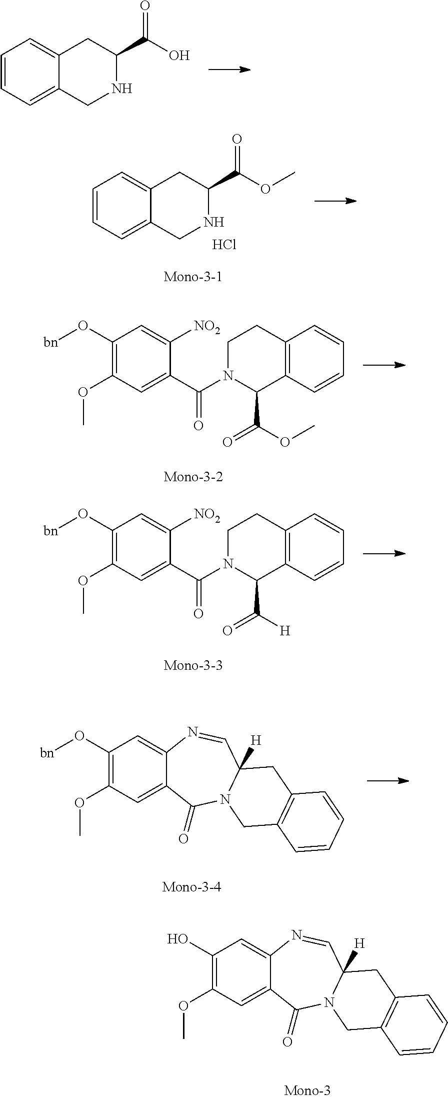

Preparation of Compound M-3-1

To a solution of (s)-(−)-1, 2, 3, 4-tetrahydroisoquinoline-3-carboxylic acid (5.0g, 28.22 mmol) in MeOH (140 mL) was added dropwise SOCl₂ (2.30 mL, 31.04 mmol) at 0° C. under N₂ atmosphere. After stirring for 21 hours at 40° C., the mixture was concentrated under reduced pressure. The residue was washed with diethyl ether (25 mL×2) to obtain compound M-3-1 (6.42 g, yield 99%).

¹H NMR (400 MHz,DMSO-d6) δ 10.02 (s, 2H), 7.27 (s, 4H), 4.60-4.56 (m, 1H), 4.39-4.29 (m, 2H), 3.82 (s, 3H), 3.19-3.12 (m, 2H); EI-MS m/z: 192 (M⁺+1).

Preparation of Compound M-3-2

To a solution of compound L-12 (9.07 g, 28.22 mmol) in anhydrous THF (50 ml) was added compound M-3-1 (6.42 g, 28.22 mmol) in THF (100 mL) and TEA (7.9 mL, 56.43 mmol) at 0° C. After stirring for 2 hours at room temperature the reaction was diluted with distilled water (500 mL) and EA (800 mL). The organic layer was dried over anhydrous Na₂SO₄, filtered, and concentrated under reduced pressure. The residue was purified by column chromatography to obtain compound M-3-2 (12.01 g, 90%). EI-MS m/z: 477 (M⁺+1).

Preparation of Compound M-3-3

To a solution of compound M-3-2 (4 g, 8.39 mmol) in anhydrous DCM (18 mL) and toluene (52 mL) was added dropwise DIBAL (16.8 mL, 16.79 mmol, 1.0M in toluene) at −78° C. under N₂ atmosphere. After stirring for 4 hours at −78° C., the reaction was quenched with MeOH (0.4 mL), 2N HCl (25 mL) at −78° C. Distilled water (100 mL) and EA (500 mL) were added thereto. The organic layer was dried over anhydrous Na₂SO₄, filtered, and concentrated under reduced pressure. The residue was purified by column chromatography to obtain compound M-3-3 (3.07 g, 82%).

EI-MS m/z: 447(M⁺+1).

Preparation of Compound M-3-4

To a solution of compound M-3-3 (3 g, 6.72 mmol) in THF (130 mL) and distilled water (86 mL) was added Na₂S₂O_{4.2}H₂O (11.3 g, 53.76 mmol) at room temperature. After stirring for 5 hours the reaction was concentrated under reduced pressure four times by using toluene as a co-solvent, thereby removing water. The obtained yellow solid was dissolved in anhydrous MeOH (220 mL), and acetyl chloride (4.8 mL, 67.19 mmol) was added thereto. After stirring for 15 minutes, the reaction mixture was adjusted to pH 7 by addition of saturated NaHCO₃ solution and diluted with distilled water (100 mL) and EA (250 mL×2). The organic layer was dried over anhydrous Na₂SO₄, filtered, and concentrated under reduced pressure. The residue was purified by column chromatography to obtain compound M-3-4 (2.48 g, 93%).

¹H NMR (400 MHz, CDCl₃) δ 7.55 (s, 1H), 7.45-7.27 (m, 10H), 6.84 (s, 1H), 5.24-5.15 (m, 2H), 5.00 (d, J=15.2, 1H), 4.56 (d, J=15.6, 1H), 3.97 (s, 3H), 3.93-3.92 (m, 1H), 3.31-3.12 (m, 2H).; EI-MS m/z: 399(M⁺+1).

Preparation of Compound M-3

To a solution of compound M-3-4 (1 g, 2.51 mmol) in anhydrous DCM (10 mL) was added methanesulfonic acid (5 mL) in DCM (10 mL) at 0° C. After stirring for 3 hours at 0° C., the mixture was quenched with NaHCO₃ solution and then diluted with distilled water (100 mL) and EA (400 mL). The organic layer was dried over anhydrous Na₂SO₄, filtered, and concentrated under reduced pressure. The residue was purified by column chromatography to obtain compound M-3 (703 mg, 91%).

¹H NMR (400 MHz, CDCl₃) δ 7.54 (s, 1H), 7.48 (d, J=4.8 Hz, 1H), 7.37-7.26 (m, 4H), 6.88 (s, 1H), 6.03 (s, 1H), 5.00 (d, J=15.6 Hz, 1H), 4.56 (d, J=15.6 Hz, 1H), 3.98 (s, 3H), 3.95-3.90 (m, 1H), 3.30-3.13 (m, 2H).

EI-MS m/z: 309(M⁺+1).

### Example 33. Preparation of Compound D-101

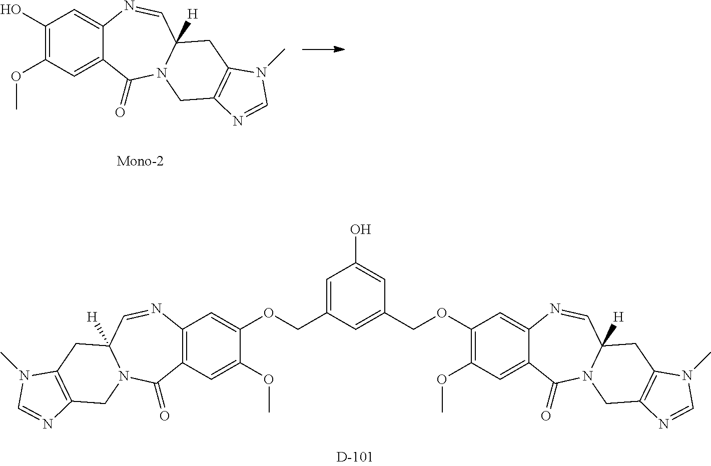

To a solution of compound Mono-2 (2.0 mg, 0.005 mmol) and compound L-10 (3.3 mg, 0.010 mmol) in DMF (1.0 mL) was added K₂CO₃ (2.0 mg, 0.012 mmol) at room temperature under N₂ atmosphere. After stirring for 3 hours, the reaction mixture was purified by prep-HPLC to obtain compound D-101 (1.2 mg, 34%).

EI-MS m/z: 743 (M⁺+1).

### Example 34. Preparation of Compound D-102

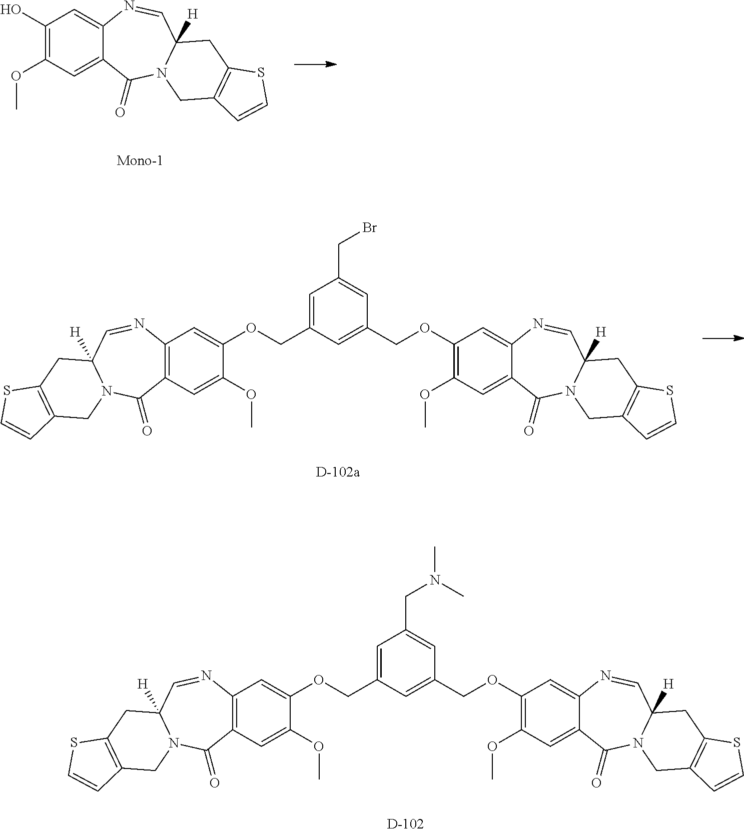

Preparation of Compound D-102a

To a solution of compound Mono-1 (100 mg, 0.318 mmol) and 1,3,5-trisbromomethyl benzene (57 mg, 0.159 mmol) in DMF (3 mL) was added K₂CO₃ (66 mg, 0.477 mmol) at room temperature under N₂ atmosphere. After stirring for 3 hours, the reaction mixture was purified by prep-HPLC to obtain compound D-102a (62 mg, 48%).

EI-MS m/z: 824 (M⁺+1).

Preparation of Compound D-102

To a solution of compound D-102a (62 mg, 0.075 mmol) in DMF (1 mL) was added 1M dimethylamine in THF (0.5 mL) at room temperature under N₂ atmosphere. After stirring for 1 hour, the reaction mixture was purified by prep-HPLC to obtain compound D-102 (39 mg, 60%).

EI-MS m/z: 788 (M⁺+1).

### Example 35. Preparation of Compound D-103

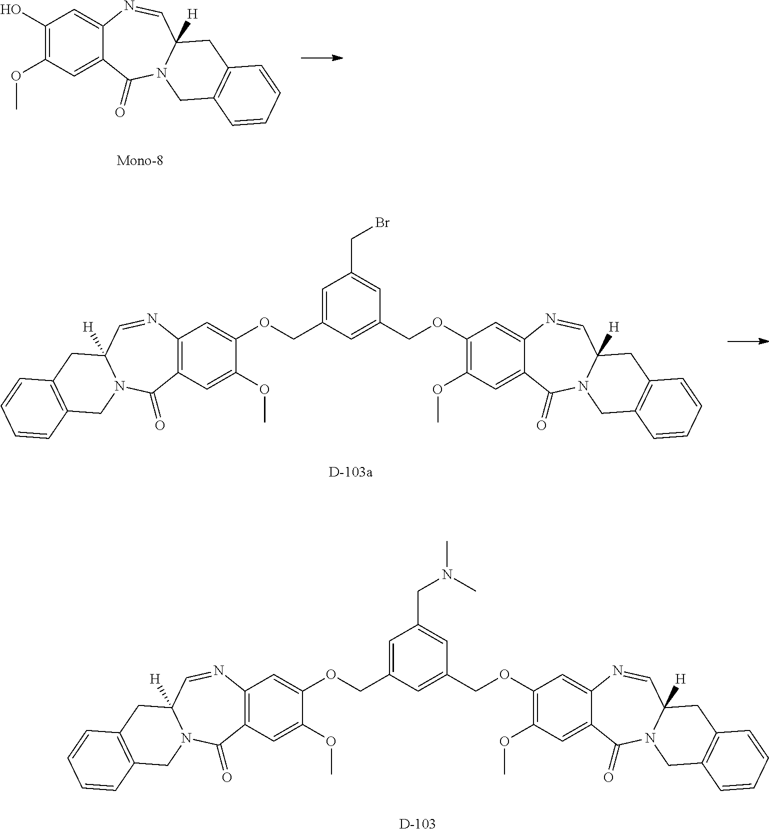

Compound D-103 was synthesized via a similar synthetic route as described in Example 34.

Preparation of Compound D-103a

Yield 42%.

EI-MS m/z: 812 (M⁺+1).

Preparation of Compound D-103

Yield 17%.

EI-MS m/z: 776(M⁺).

### Example 36. Preparation of Compound MMAF-OMe

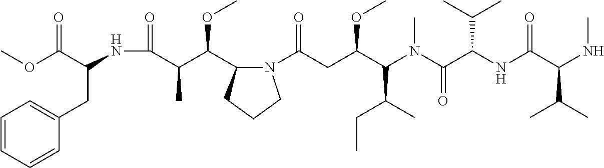

MMAF-OMe was synthesized by a similar synthetic method as described in the U.S. Pat. Nos. 7,423,116 and 7,498,298, and International Patent Application Publication WO2002/088172, each of which is incorporated herein by reference in their entirety.

### Example 37. Preparation of Compound Int-TG4

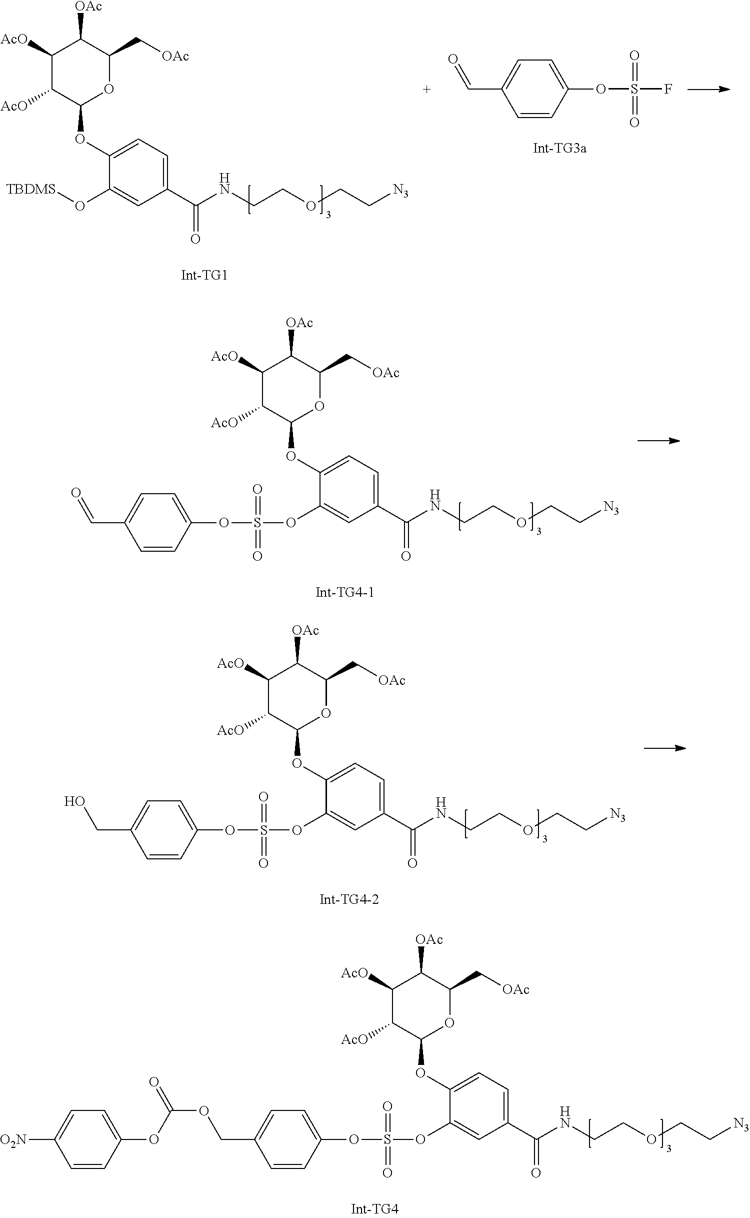

Preparation of Compound Int-TG4-1

To a solution of compound Int-TG1 (100 mg, 0.13 mmol) and compound Int-TG3a (26 mg, 0.13 mmol) in anhydrous MeCN (3 mL) were added DBU (4 μL, 25 μmol). The mixture was stirred at room temperature for 1 hour and was washed with distilled water (10 mL) and EA (10 mL×2). The organic layer was dried over anhydrous Na₂SO₄, filtered, and concentrated under reduced pressure. The residue was purified by column chromatography to obtain compound Int-TG4-1 (103 mg, 94%).

EI-MS m/z: 869(M⁺).

Preparation of Compound Int-TG4-2

To a solution of compound Int-TG4-1 (103 mg, 0.12 mmol) in THF (8 mL) was added NaBH₄ (9 mg, 0.24 mmol) at 0° C. under N₂ atmosphere. After stirring at room temperature for 2 hours, distilled water (10 mL) and EA (10 mL×2) were added. The organic layer was dried over anhydrous Na₂SO₄, filtered, and concentrated under reduced pressure to obtain compound Int-TG4-2 (101 mg, 98%).

EI-MS m/z: 871(M⁺).

Preparation of Compound Int-TG4

To a solution of compound Int-TG4-2 (47 mg, 54 μmol) in DMF (2 mL) was added bis(4-nitrophenyl) carbonate (25 mg, 81 μmol) and DIPEA (14 μL, 81 μmol) at room temperature under a nitrogen atmosphere. The mixture was stirred overnight at room temperature. Distilled water (10 mL) and EA (10 mL×2) were added, the organic layer was dried over anhydrous Na₂SO₄, filtered, and concentrated under reduced pressure. The residue was purified by column chromatography to obtain compound Int-TG4 (53 mg, 94%).

EI-MS m/z: 1036(M⁺).

### Example 38. Preparation of Compound T-Int-1 (B-3)

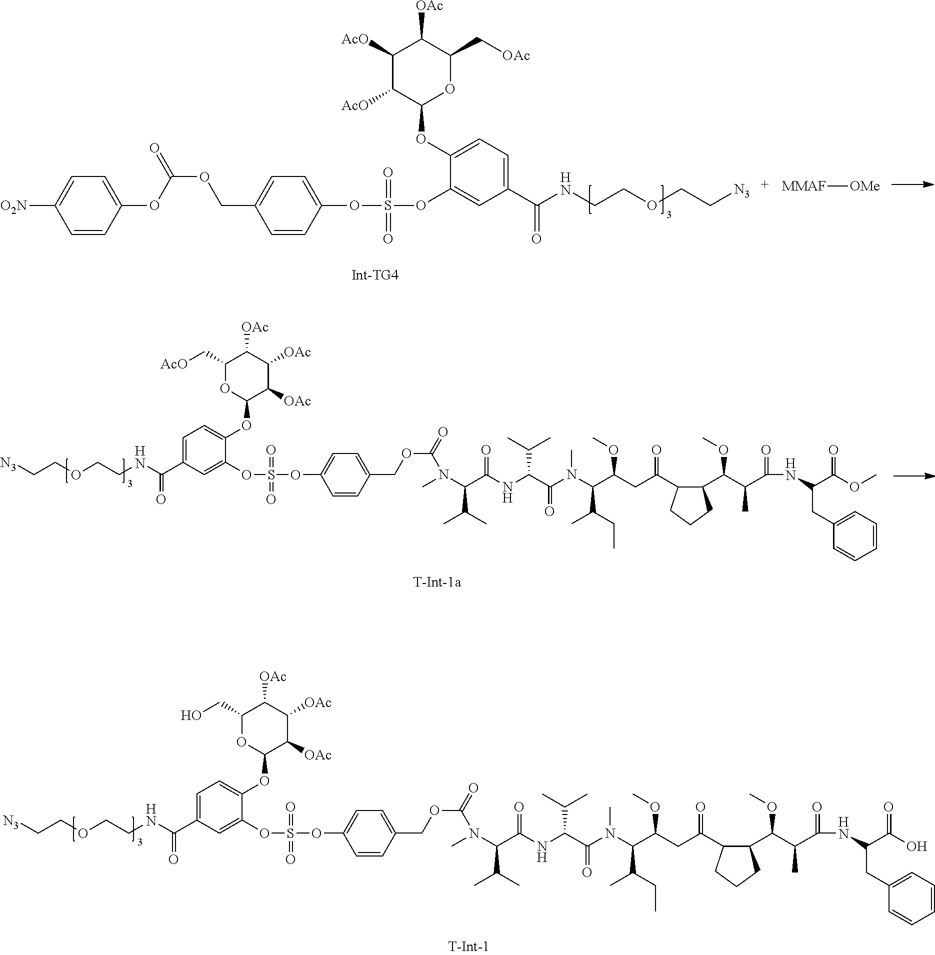

Preparation of Compound T-Int-1a

To a solution of compound Int-TG4 (65 mg, 0.063 mmol) and MMAF-OMe (52 mg, 0.069 mmol) in DMF (1 mL) was added HOBt (2 mg, 0.013 mmol), DIPEA (12 μL, 0.069 mmol), and pyridine (330 μL) at room temperature under N₂ atmosphere. After stirring overnight, the mixture was adjusted to pH 2 to 3 with 1N HCl and extracted with EA (8 mL×2). The organic layer was washed distilled water (8 mL) and brine (12 mL, dried over anhydrous Na₂SO₄, filtered, and concentrated under reduced pressure. The residue was subjected to column chromatography to obtain compound T-Int-1a (73 mg, 71%).

EI-MS m/z: 1644(M⁺¹).

Preparation of Compound T-Int-1

A homogeneous solution of T-Int-1a (73 mg, 0.044 mmol) in anhydrous MeOH (1.5 mL) at 0° C. under N₂ atmosphere was treated with LiOH (14 mg, 0.333 mmol) and distilled water (1.5 mL) and stirred to room temperature for 2 hours. The reaction was quenched with 1N HCl (2 mL) and reaction mixture was purified by Prep HPLC chromatography to give the title compound T-Int-1 (45 mg, 69%).

EI-MS m/z: 1462(M⁺).

### Example 39. Preparation of Compound T-Int-2

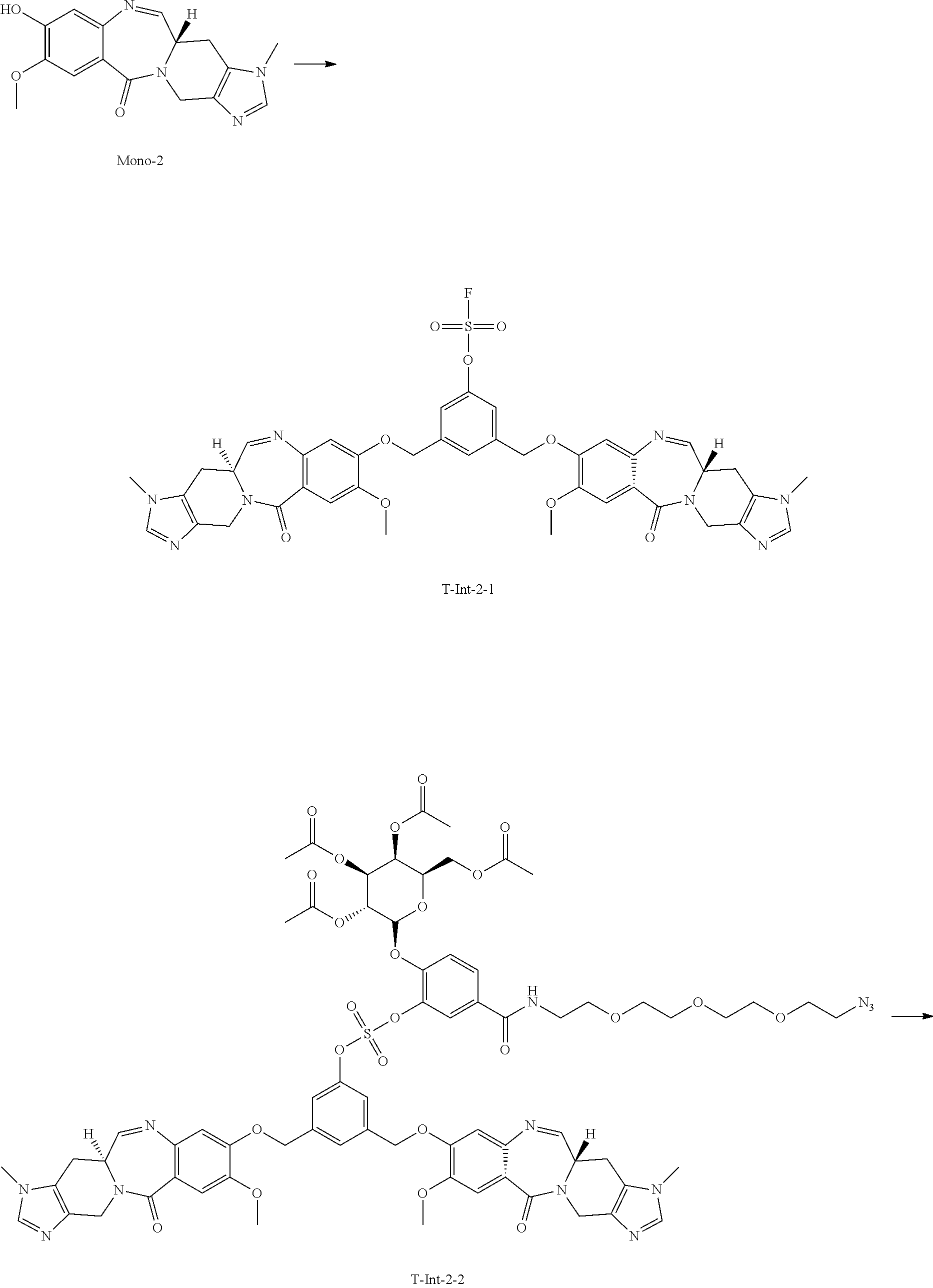

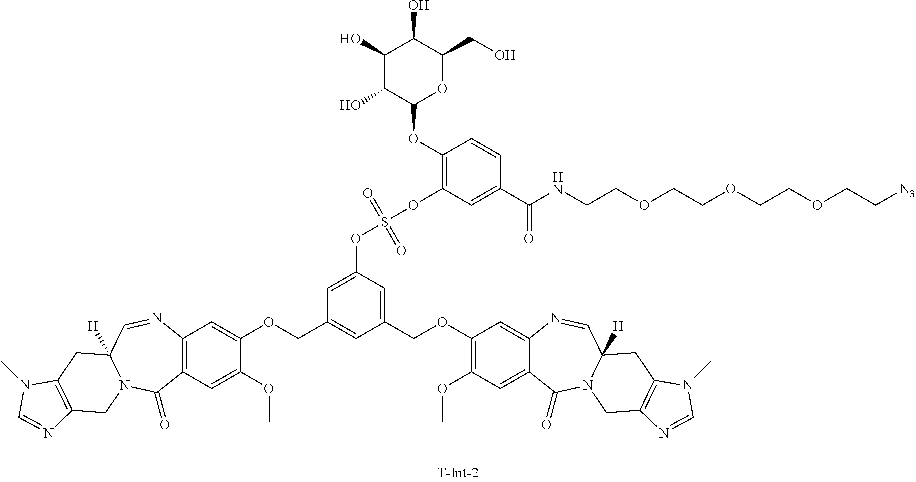

Preparation of Compound T-Int-2-1

To a solution of compound Mono-2 (14 mg, 0.04 mmol) and compound L-9 (8.0 mg, 0.02 mmol) in DMF (0.6 mL) was added K₂CO₃ (9.3 mg, 0.07 mmol) at 30° C. under N₂ atmosphere. After stirring for 3 hours, the reaction mixture was purified by prep-PLC to obtain compound T-Int-2-1 (5.4 mg, 29%). ELMS m/z: 825 (M⁺+1).

Preparation of Compound T-Int-2-2

To a solution of compound T-Int-2-1 (7.5 mg, 0.01 mmol) and compound Int-TG1 (14 mg, 0.02 mmol) in MeCN (0.5 mL) and DMF (0.5 mL) was added BEMP (1 μL, 0.004 mmol) at room temperature under N₂ atmosphere. After stirring for 5 hours at room temperature, the reaction mixture was purified by HPLC to obtain compound T-Int-2-2 (67 mg, 83%). EI-MS m/z: 1490 (M⁺+1).

Preparation of Compound T-Int-2

To a solution of compound T-Int-1-2 (8.1 mg, 0.01 mmol) in MeOH (1 mL) and DCM (0.1 mL) was added K₂CO₃ (5.6 mg, 0.04 mmol) under N₂ atmosphere. After stirring for 1 hour at 0° C., the reaction mixture was purified by HPLC to obtain compound T-Int-2 (5.5 mg, 76%). ELMS m/z: 1322 (M⁺+1).

### Example 40. Preparation of Compound T-Int-3

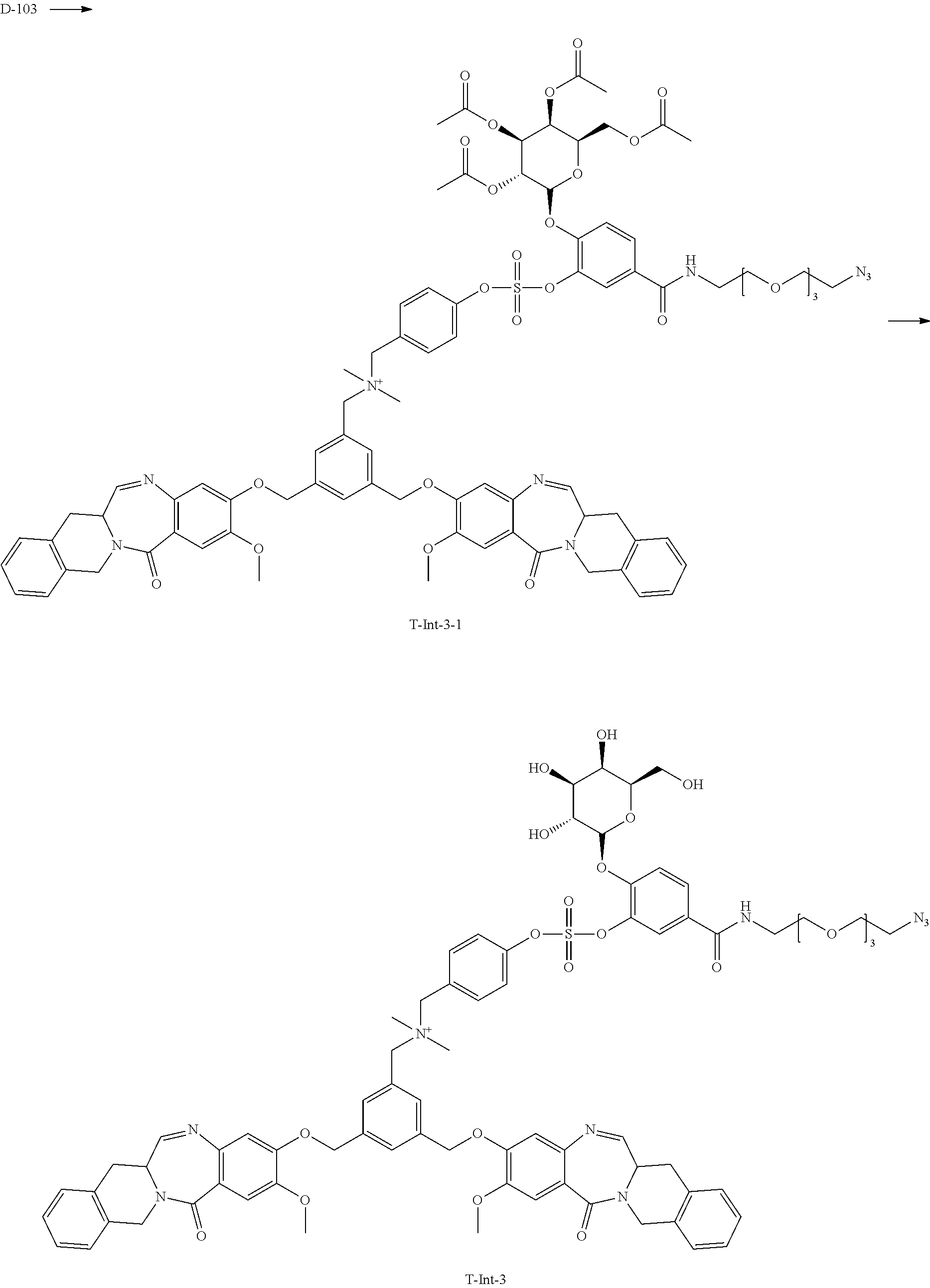

Preparation of Compound T-Int-3-1

To a solution of compound D-103 (22.8 mg, 0.03 mmol) and compound Int-TG3-3 (27.4 mg, 0.03 mmol) in DMF (2 mL) was added DIPEA (12 uL, 0.07 mmol) at 40° C. under N₂ atmosphere. After stirring for 5 hours at room temperature, the reaction mixture was purified by prep-HPLC to obtain compound T-Int-3-1 (28.9 mg, 71%).

EI-MS m/z: 1630 (M⁺+1).

Preparation of Compound T-Int-3

To a solution of Compound T-Int-3-1 (28.9 mg, 0.02 mmol) in MeOH (2 mL) was added K₂CO₃ (12.2 mg, 0.09 mmol) under N₂ atmosphere. After stirring for 1 hour at 0° C. under N₂ atmosphere, the reaction mixture was purified by prep-HPLC to obtain compound T-Int-3 (18.4 mg, 71%).

EI-MS m/z: 1462 (M⁺+1).

### Example 41. Preparation of Compound T-1 and T-2

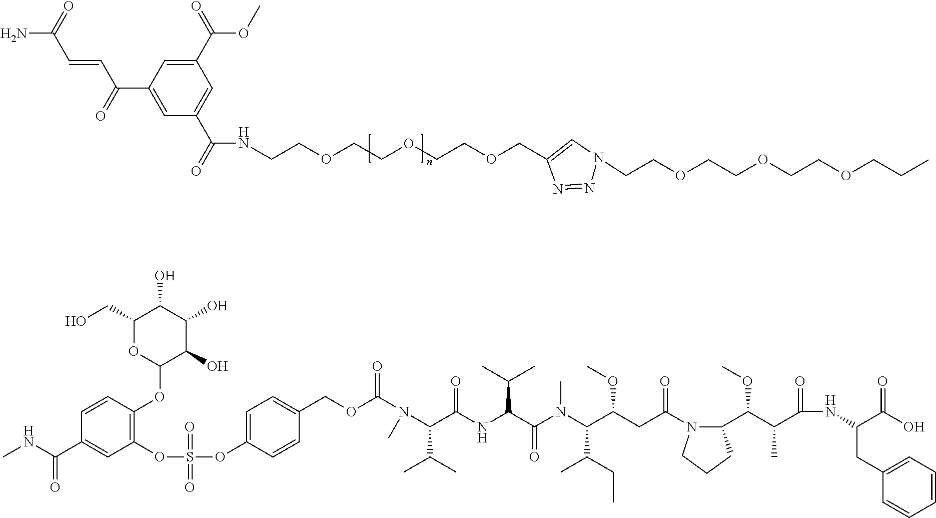

To a homogeneous solution of T-Int-1 (2 mg, 1.37 μmol) and AMA-5 (2 mg, 4.11 μmol) in DMSO (4.5 mL) was added (BimC₄A)₃ (4.5 mg, 5.48 μmol), CuBr (6.42 mg, 44.8 μmol) at room temperature under a nitrogen atmosphere, and the resulting mixture was stirred for 10 min. The reaction mixture was purified by Prep HPLC chromatography to give the title compound T-1 (0.4 mg, 15%). EI-MS m/z: 954 (M⁺⁺1).

Compound T-2 was synthesized via a similar manner to the preparation method of the compound T-1.

Yield 33%, white solid. ELMS m/z: 976 (M/2⁺+1).

### Example 42. Preparation of Compound T-3

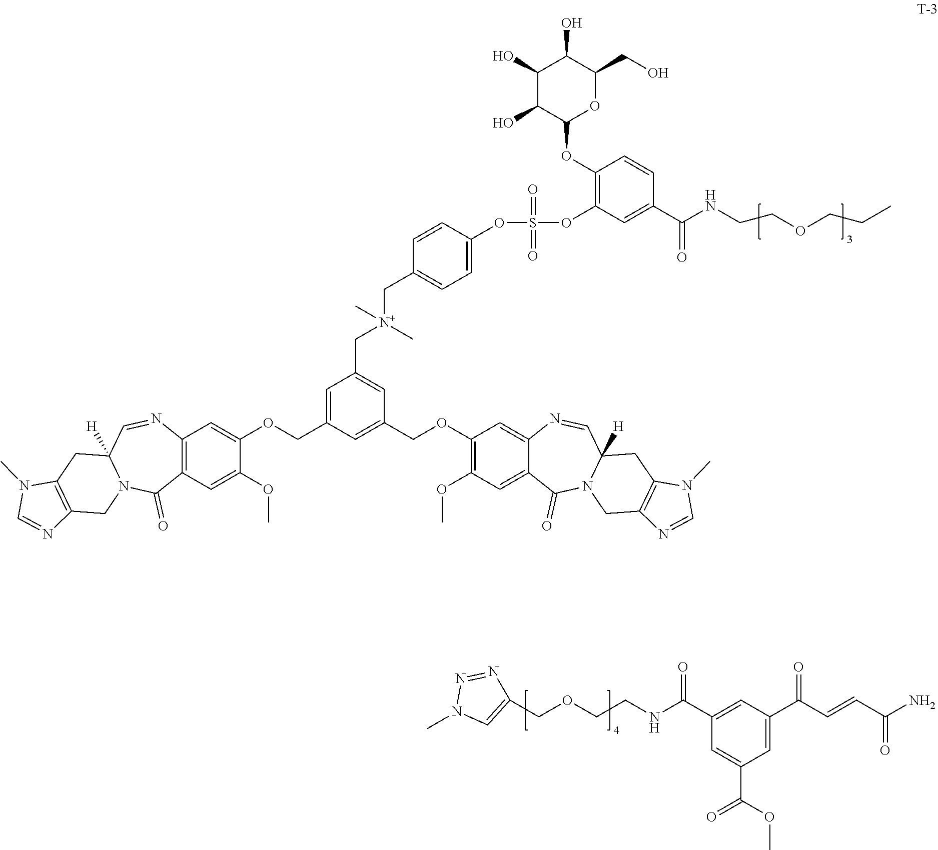

Compound T-3 was synthesized via a similar manner to the preparation method of the compound T-2 of Example 41.

Yield 40%, white solid. EI-MS m/z: 906 (M⁺+1).

### Example 43. Preparation of Compound T-4

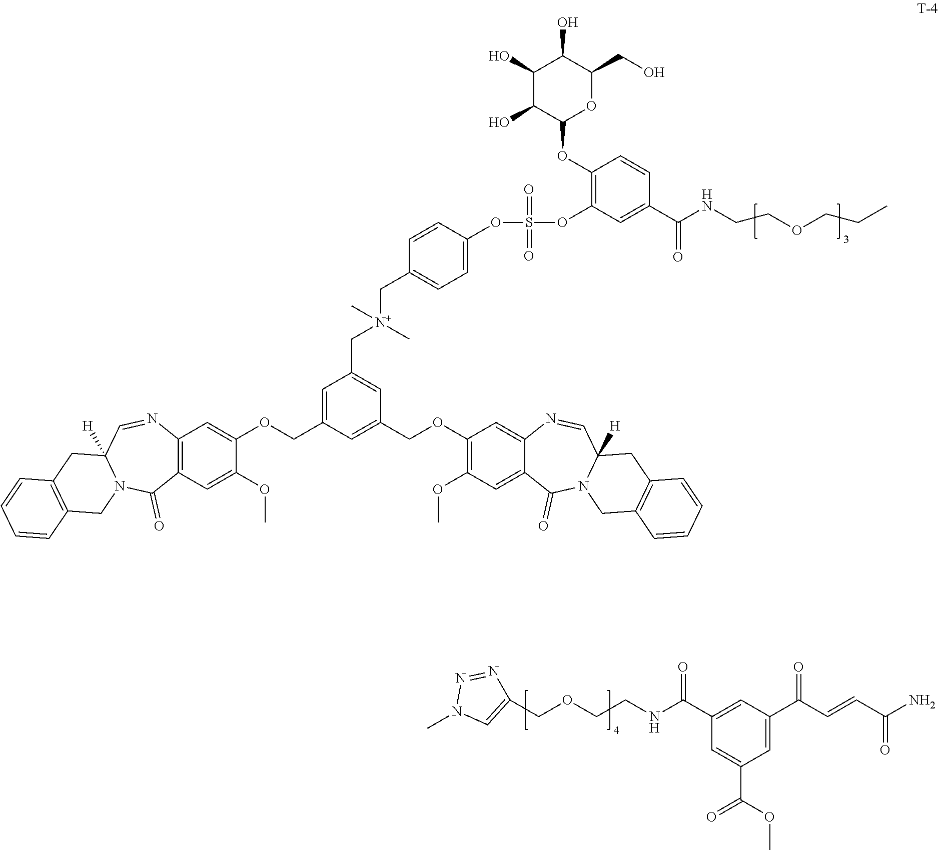

Compound T-4 was synthesized via a similar manner to the preparation method of the compound T-2 of Example 41.

Yield 77%, white solid. EI-MS m/z: 977 (M⁺+1).

### Example 44. Preparation of Compound T-5

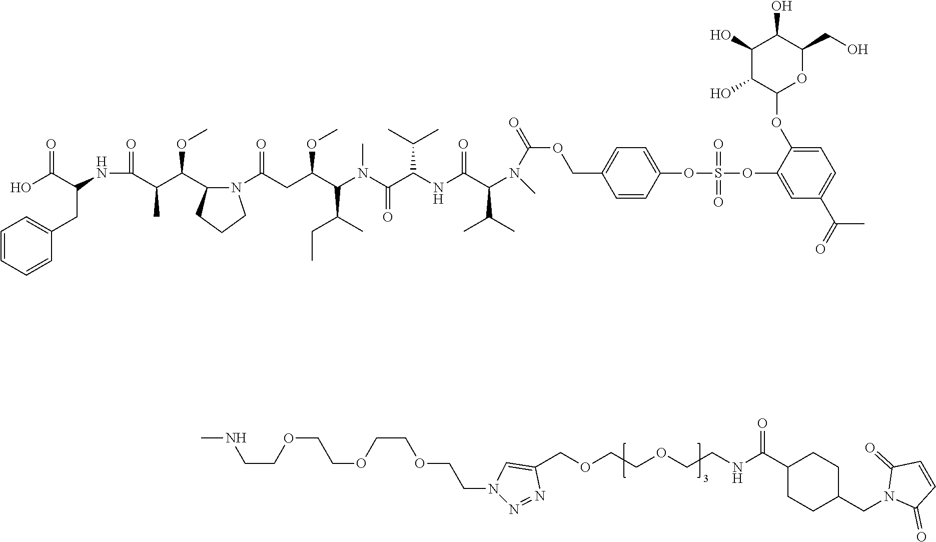

A homogeneous solution of T-Int-1 (2.5 mg, 0.0017 mmol) and Mal-1 (2.3 mg, 0.0051 mmol) in DMSO (3461 μL) at room temperature under N₂ atmosphere was treated (BimC₄A)₃ in DMSO (1368 μL, 0.0068 mmol) and stirred for 10 minutes. CuBr (171 μL, 0.017 mmol) in DMSO was added to the reaction mixture and the resulting mixture was stirred for 5 minutes. The reaction mixture was purified by preparative HPLC (Column: Innoval ODS-2 10 um, 100 Å, 21.2×250 mm; flow rate: 15 mL/min, A buffer 0.1% Formic acid in water/B buffer 0.1% Formic acid in ACN, method gradient, solvent A: solvent B 95: 5 to 5: 95, 1 hour, wavelength 214 nm) to obtain compound 11 (2.1 mg, 64%) as white solid.

EI-MS m/z: 957 (M/2⁺+1).

### Example 45. Preparation of Compound T-6

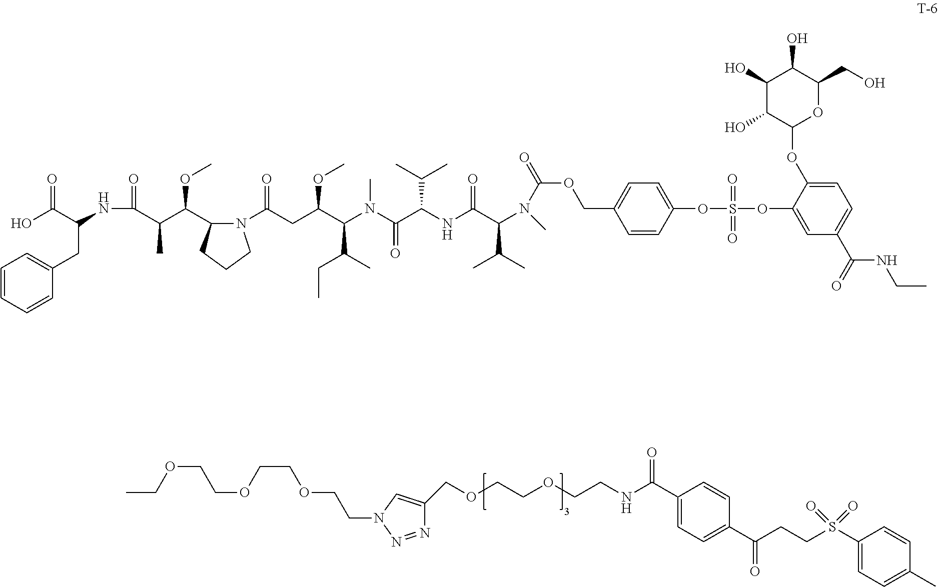

Compound T-6 was synthesized via a similar manner to the preparation method of the compound T-5 of Example 44.

Yield 70%, white solid. EI-MS m/z: 1007 (M/2⁺¹).

### Example 46. Preparation of Compound T-7

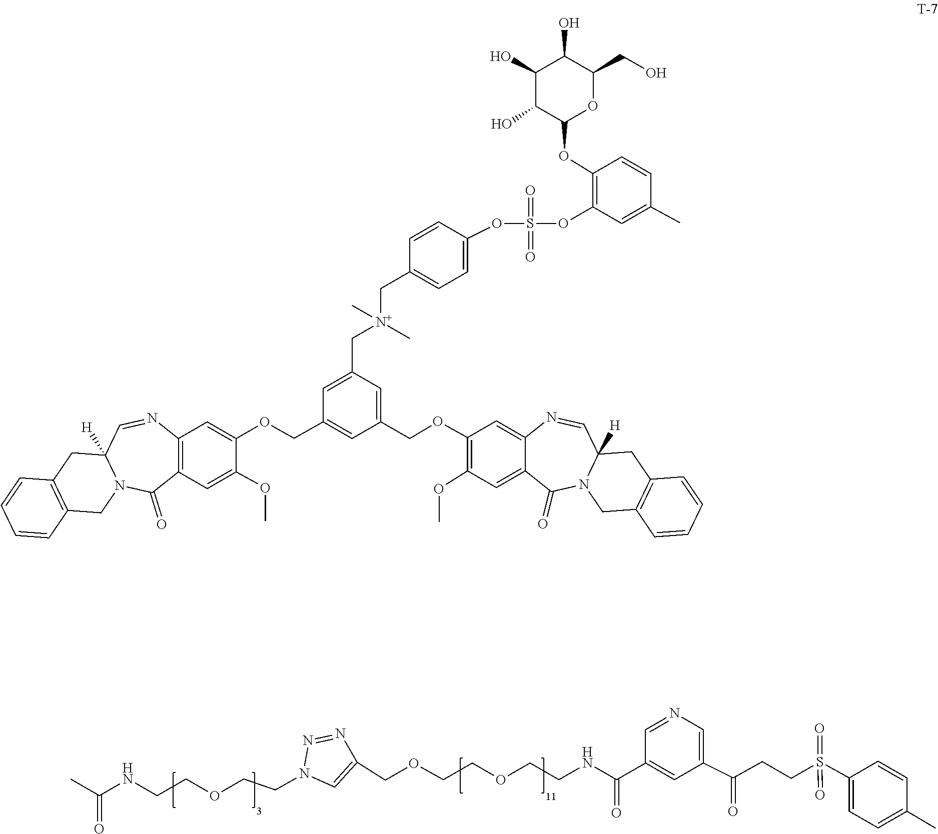

Compound T-7 was synthesized via a similar manner to the preparation method of the compound T-5 of Example 44.

Yield 53%, white solid. EI-MS m/z: 1181 (M/2⁺¹).

### Example 47. Preparation of Compound T-8

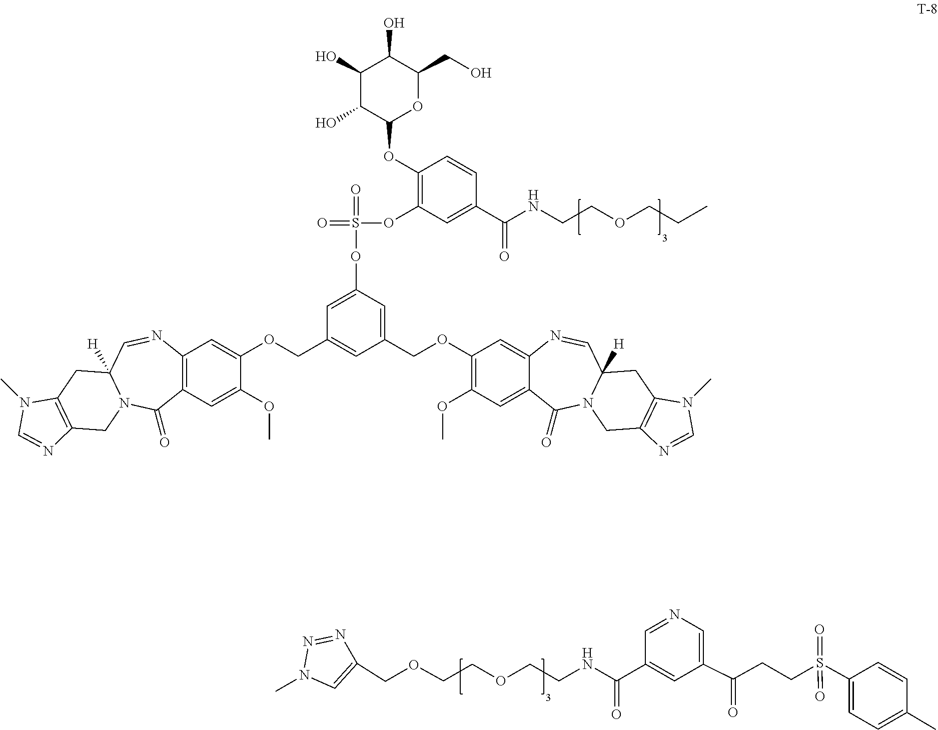

Compound T-8 was synthesized via a similar manner to the preparation method of the compound T-5 of Example 44.

Yield 15%, white solid. ELMS m/z: 935 (M/2⁺¹).

### Example 48. Preparation of Compound T-9

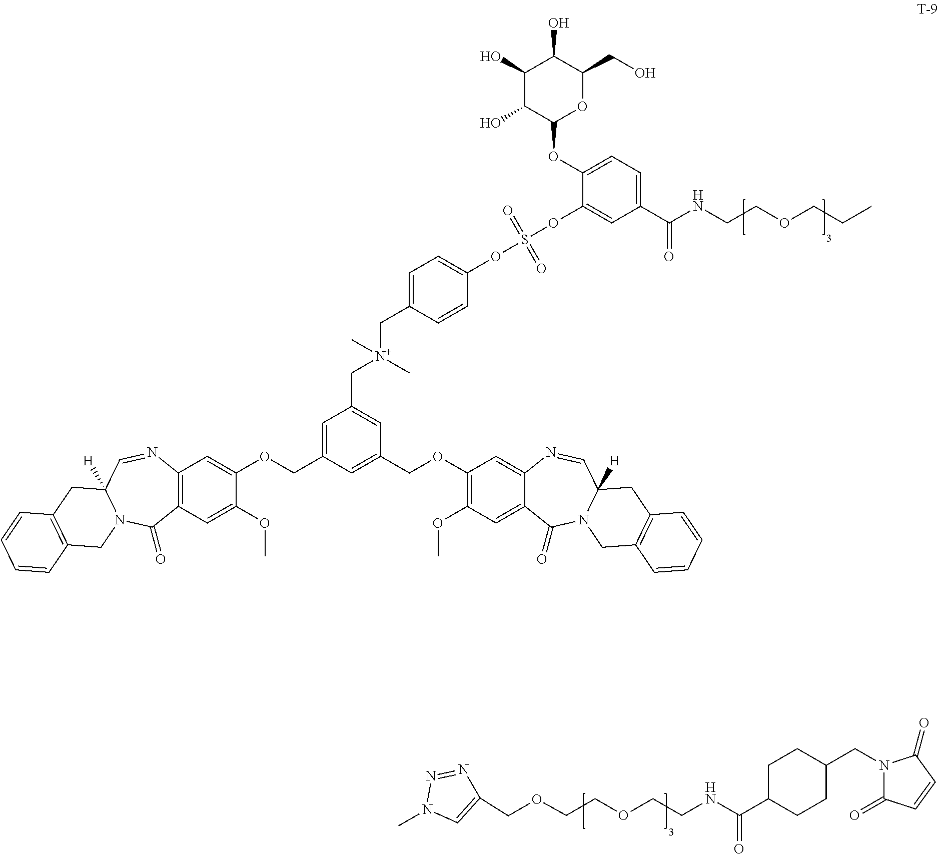

Compound T-9 was synthesized via a similar manner to the preparation method of the compound T-5 of Example 44.

Yield 61%, white solid. ELMS m/z: 957 (M/2⁺¹ ).

### Example 49. Preparation of Compound T-10

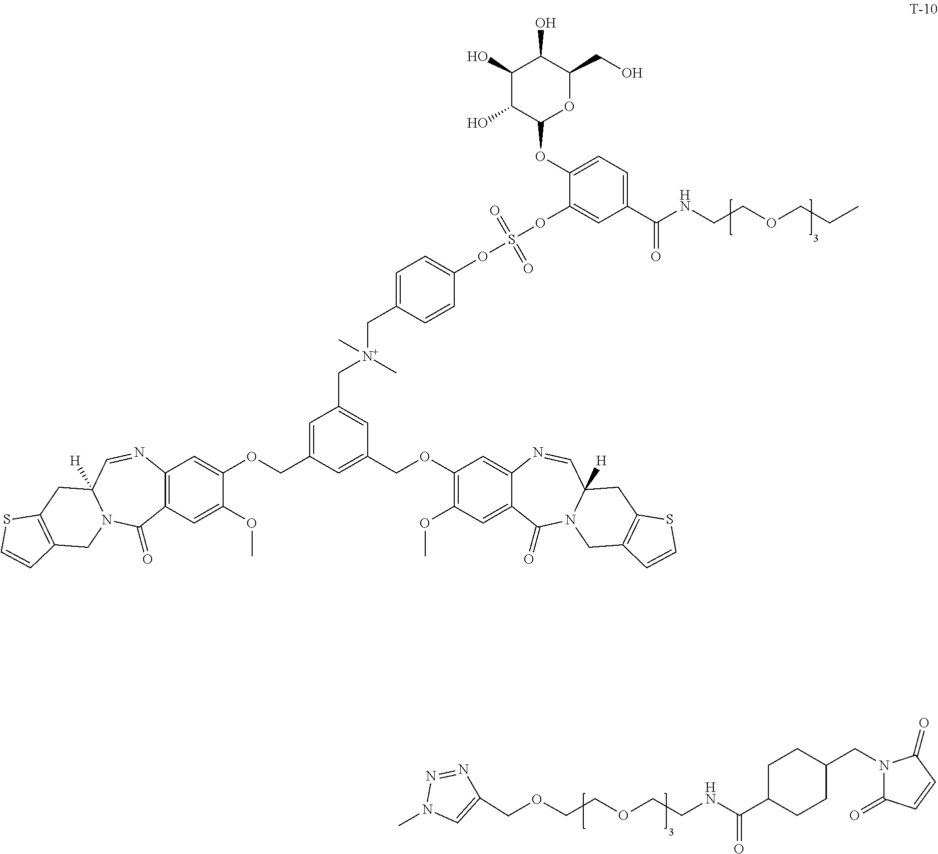

Compound T-10 was synthesized via a similar manner to the preparation method of the compound T-5 of Example 44.

Yield 65%, white solid. EI-MS m/z: 963(M/2⁺¹).

### Example 50. Preparation of Compound “BG-SIG”

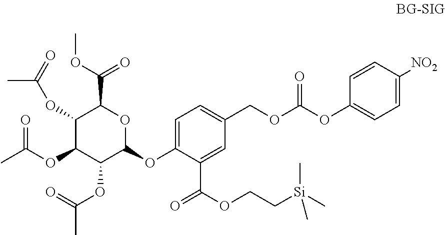

BG-SIG was synthesized by a similar route as described in U.S. Pat. No. 10,383,949, which is incorporated herein by reference in its entirety.

### Example 51. Preparation of Compound T-Int-4

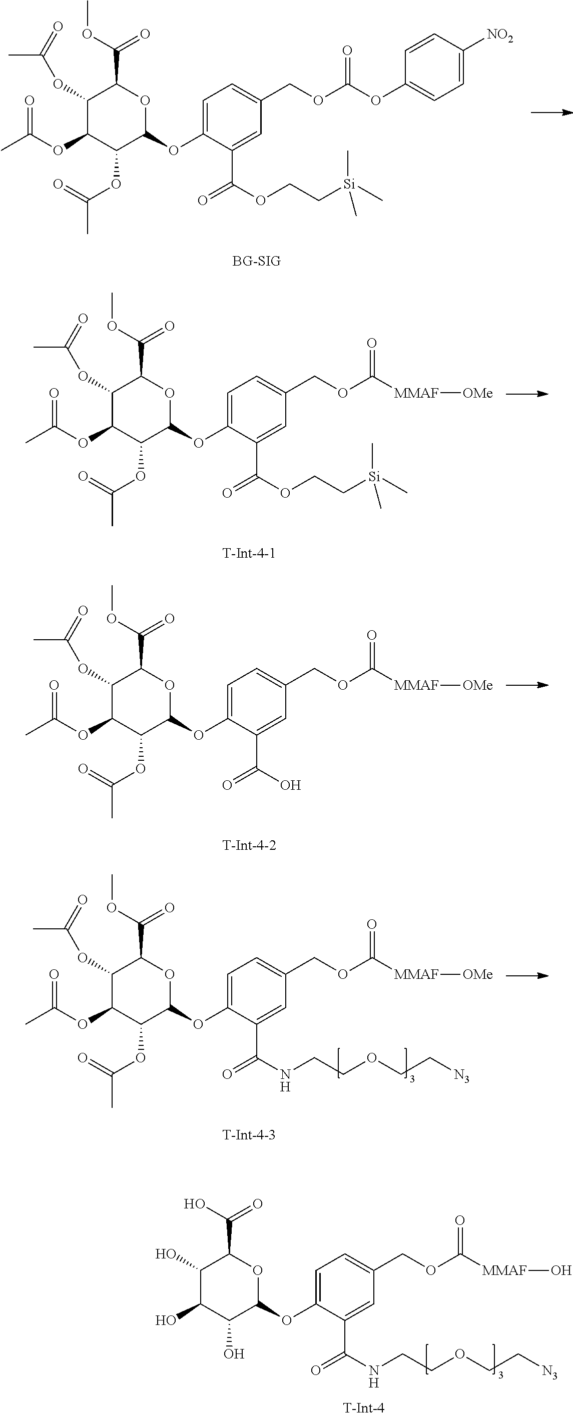

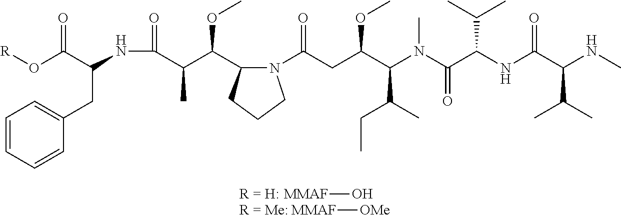

T-Int-4-1, T-Int-4-2, and T-Int-4-3 were synthesized by a similar route as described in in U.S. Pat. No. 10,383,949, which is incorporated herein by reference in its entirety.

Preparation of Compound T-Int-4-1

Yield 82%; EI-MS m/z: 1357 (M⁺¹).

Preparation of Compound T-Int-4-2

Yield 76%; EI-MS m/z: 1257 (M⁺¹).

Preparation of Compound T-Int-4-3

Yield 75%; EI-MS m/z: 1457 (M⁺¹).

Preparation of Compound T-Int-4

Compound T-Int-4 was synthesized via a similar manner to the preparation method of the compound T-Int-1 of Example 38.

Yield 88X %; EI-MS m/z: 1303 (M⁺¹).

### Example 52. Preparation of Compound T-11

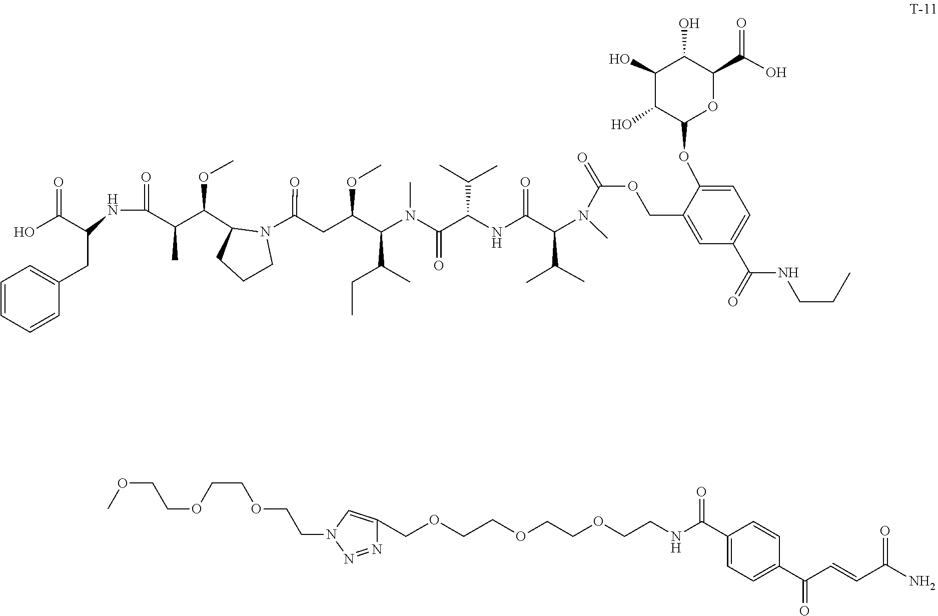

Compound T-11 was synthesized via a similar manner to the preparation method of the compound T-5 of Example 44.

Yield 80%, white solid. EI-MS m/z: 846(M/2″).

### Example 53. Preparation of Compound T-12

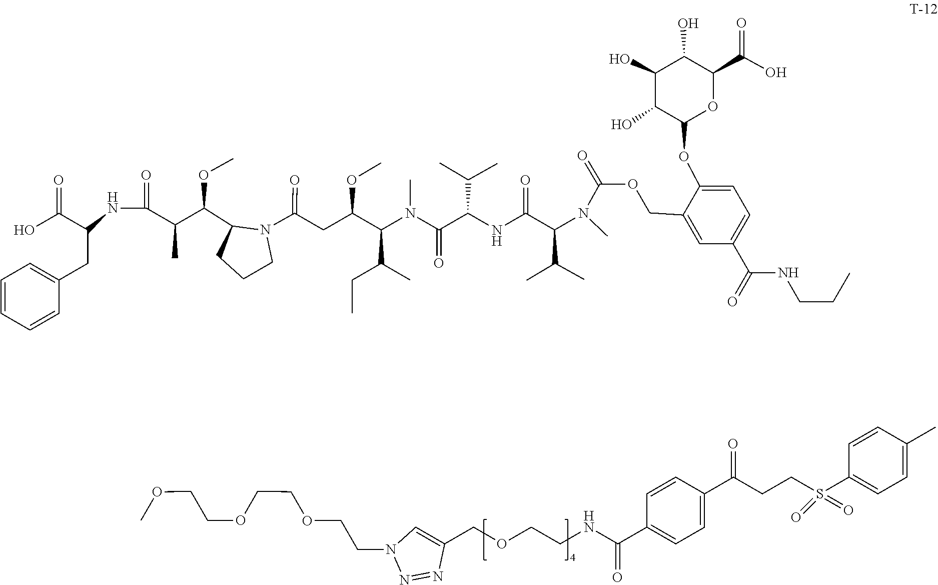

Compound T-12 was synthesized via a similar manner to the preparation method of the compound T-5 of Example 44.

Yield 71%, white solid. EI-MS m/z: 925(M/2⁺¹).

### Example 54. Preparation of Compound T-13

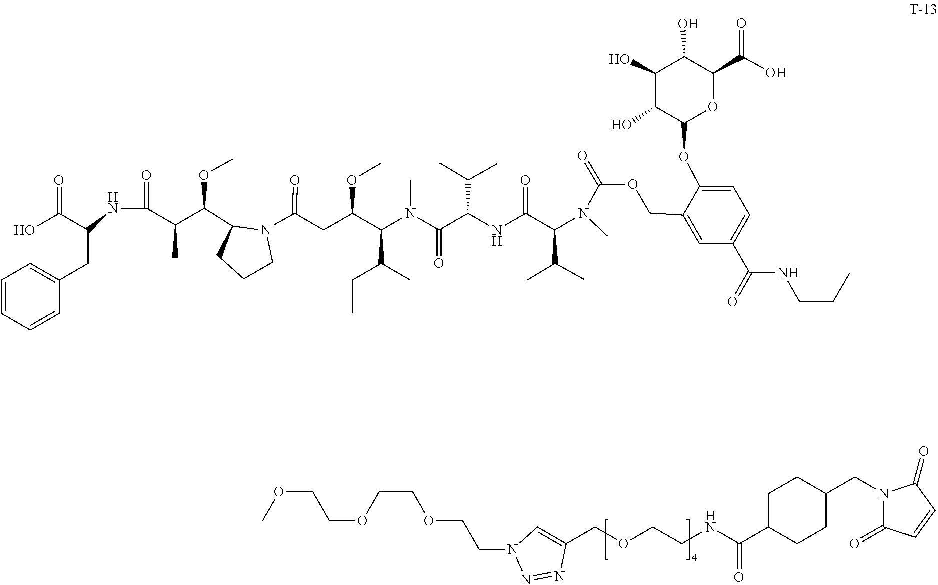

Compound T-13 was synthesized via a similar manner to the preparation method of the compound T-5 of Example 44.

Yield 56%, white solid. EI-MS m/z: 877(M/2⁺¹).

### Example 55. Preparation of Compound T-14

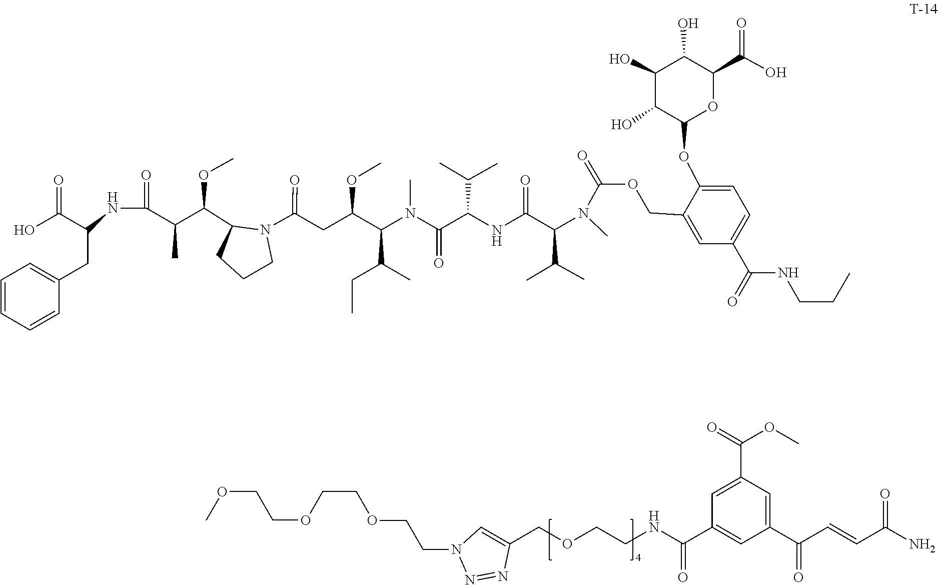

Compound T-14 was synthesized via a similar manner to the preparation method of the compound T-5 of Example 44.

Yield 70%, white solid. EI-MS m/z: 897(M/2⁺¹).

### Example 56. Preparation of Compound “MPS-1”

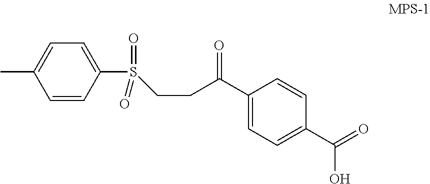 [SMILES: Cc1ccc(S(=O)(=O)CCC(=O)c2ccc(C(=O)O)cc2)cc1]

Compound MPS-1 was synthesized in a way similar to that described in Example 20.

¹H NMR (400 Hz, CDCl3) δ8.04-7.99 (m, 4H), 7.81 (d, J=8.4 Hz, 2H), 7.46 (d, J=8.4 Hz, 2H), 3.63 (t, J=7.2 Hz, 2H), 3.41 (t, J=7.2 Hz, 2H), 2.44 (s, 3H). ELMS m/z: 333 (M⁺+1).

### Example 57. Preparation of Compound “Reference A”

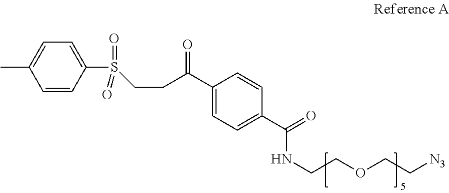

Reference A was synthesized via a similar synthetic route as described in Example 20.

EI-MS m/z: 621 (M⁺+1).

### Example 58. Preparation of Compound “N-Ac-Cys-AMA-9c”

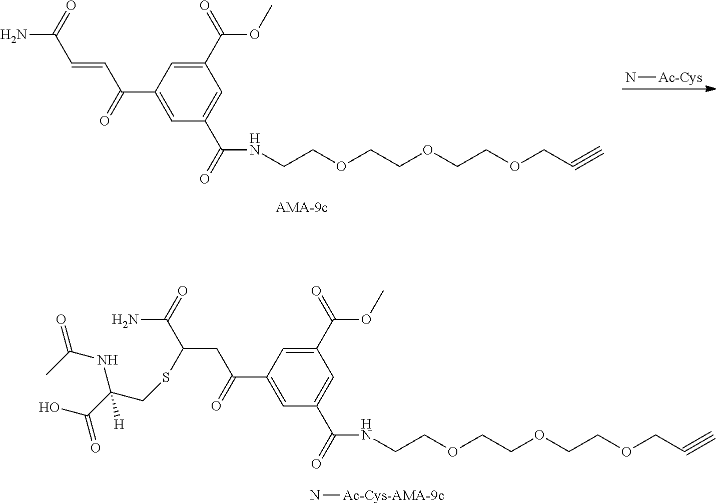

A homogeneous solution of AMA-9c (11 mg, 0.025 mmol) in PBS buffer pH7.4 (2 mL) and DMSO (0.2 mL) at room temperature under N₂ atmosphere was treated with N-Ac-cysteine (4.2 mg, 0.026 mmol) and stirred for 1.5 hrs. The reaction mixture was used in-situ for next step after check LC-Mass.

EI-MS m/z: 610 (M⁺).

### Example 59. Preparation of Compound “N-Ac-Cys-AMA-10”

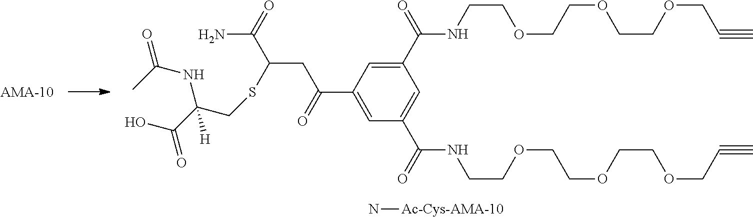

Compound N-Ac-Cys-AMA-10 was synthesized via a similar manner to the preparation method of the compound N-Ac-Cys-AMA-9c of Example 59.

Biological Testing

### Example 60. Preparation of Conjugates

Reduction/Oxidation of Antibodies for Conjugation: Cysteine engineered monoclonal antibodies were reduced with about a 20-50 fold excess of TCEP (tris(2-carboxyethyl)phosphine hydrochloride or DTT (dithiothreitol) in 4 mM Tris pH 7.3 with 1 mM EDTA for 1 hours at 37° C. The reduced thiomab was diluted and loaded onto a PD-10 column in PBS. The column was eluted with 10 mM PBS pH 7.3. The eluted reduced thiomab was re-established by air oxidation. The thiol/Ab value was checked by determining the reduced antibody concentration form the absorbance at 280 nm of the solution and the thiol concentration by reaction with DTNB (Aldrich, CAS No D8130) and determination of the absorbance at 412 nm.

Conjugation Method 1:

The compound T-1 obtained in Example 41 (3.80 μL, 3.0 mmol, as linker-toxin intermediate) in DMSO was treated with the reduced, reoxidized antibody (45 μL, 0.053 mmol) and agitated gently for 3 hours at room temperature. Sodium borohydride (3.80 μL, 300 mmol) was added to a solution of the reaction mixture and incubated at 37° C. for 1 hour to block a reversible deconjugation reaction. The conjugation mixture was loaded and eluted through PD-10 column to remove excess drug-linker intermediate and other impurities.

Conjugation Method 2:

After the reduction and reoxidation reaction, the antibody was dissolved in PBS. A solution of compound T-3, and T-4 obtained in Example 42, and 43 (8.86 μL, 3.0 mmol, as linker-toxin intermediate) in DMSO was treated with the reduced, reoxidized antibody (70 μL, 0.053 mmol) and agitated gently for 3 hours at room temperature. Hydroxylamine (8.86 μL, 1,500 mmol) was added to a solution of the reaction mixture and incubated at 37° C. for 8 hours to block a reversible deconjugation reaction. The conjugation mixture was loaded and eluted through PD-10 column to remove excess drug-linker intermediate and other impurities.

Compounds T-1, T-3, and T-4 obtained in Examples 41, 42, and 43 were used to perform conjugation reaction to a thiol group of engineered cysteine of trastuzumab (anti-HER2), thereby preparing T-1-AB, T-3-AB, and T-4-AB as thiomab drug conjugates (TDC), respectively, with reference to methods presented in document. [see Nature Biotechnology, 2008, 26, 925-932, Bioconjugate Chem., 2013, 24, 1256-1263, Bioconjugate Chem., 2016, 27, 1324-1331, Bioconjugate Chem. 2014, 25, 460-469]. DAR (drug to antibody ratio) of conjugated antibody was analyzed by HIC and results of the analysis were shown in Table 1.

Conjugation Method 3: Maleimide Conjugation Protocol.

After the reduction and reoxidation reaction, the resulting antibody was treated with 3.5 equiv of the compound T-10 as 3 mM stock solution in DMSO to achieve 7.5% (v/v) total organic. The reaction was allowed to stand for 1 hours at 40° C. then excess drug-linker intermediate and other impurities were removed by using PD-10 column. Final samples were concentrated to −5 mg/ml protein.

TABLE 1
Antibody-Drug Conjugates (ADCs)

| ADCs | DAR | Conjugation Method | Linker-Toxin, Example |
|---|---|---|---|
| T-1-AB | 1.76 | 1 | T-1, Example 41 |
| T-3-AB | 0.5 | 2 | T-3, Example 42 |
| T-4-AB | 0.42 | 2 | T-4, Example 43 |

### Example 61. Cell Cytotoxicity of Antibody-Drug Conjugate

NCI-N87 cancer cells were seeded in 96-well plates at a density of 5,000 cells per well in 100 μL of medium, and cultured for 24 hours. T-DM1 was treated by serial dilutions of 1:5 from 50 nM to 0.000128 nM. After 72 hours of incubation, 0.2 mL of 3-(4,5-dimethylthiazol-2-yl)-2,5-diphenyltetrazolium bromide (MTT) dye, dissolved in PBS buffer solution (5 mg/mL), was added to each well of the plates. The formazans formed by reduction of the MTT dye by mitochondrial oxidoreductases in the living cells were dissolved in DMSO, and measured using the absorbance at 550 nm.

### Example 62. Studies of Chemoselectivity of the Reactions of AMA-9c with N-Ac-Cys, N-Ac-Lys, and N-Ac-Tyr

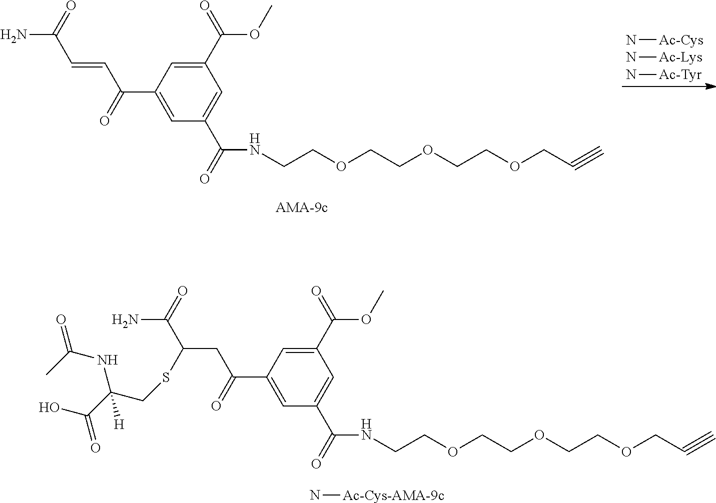

To a homogeneous solution of AMA-9c (10 μL, stock solution 10 mmol) in PBS buffer (870 μL) and DMSO (90 uL) were added N-acetyl-L-lysine (10 μL, stock solution 10 mmol), N-acetyl-L-tyrosine (10 stock solution 10 mmol), and N-acetyl-L-cysteine (10 stock solution 10 mmol) and stirred for 1 hour. The chemoselectivity of the reaction of AMA-9c was checked using the LC-MS. Reaction of AMA-9c in an equimolar mixture of N-acetyl-L-lysine, N-acetyl-L-tyrosine, and N-acetyl-L-cysteine shows complete chemoselectivity for the thiol group of cysteine.

### Example 63. Chemical Stability (Hydration Stability) Studies of AMA-9c

This study was conducted to check the stability of AMA-9c. The compound AMA-9c was dissolved in DMSO and mixed with PBS (pH 7.4) buffer solution to prepare a solution having a concentration of 500 μM (5% DMSO). MPS used as a standard material was prepared as a solution to have a concentration of 500 μM in PBS buffer solution. 420 μL of the buffer solution, and 140 μL of the compound AMA-6 solution and 140 μL of MPS solution were mixed, thereby preparing a reaction mixture in a total amount of 700 μL. The reaction mixture was incubated at room temperature while blocking the light. The reaction mixture was aliquoted on day 0 (before the reaction) and on 1 day, 2 days, 4 days, and 7 days after the reaction, wherein each aliquot amount was 70 μL. Then, the remaining compound AMA-9c and MPS were quantitated by HPLC analysis, indicating stability of AMA-9c in PBS buffer (see FIG. 2).

### Example 64. Plasma Stability Studies of N-Ac-Cys-AMA-10

Compound N-Ac-Cys-AMA-10 and methyl phenyl sulfone (used as a standard material) were dissolved in DMSO to make a concentration of 30 mM. Then, each of human plasma (Biochemed 752PR—SC-PMG) and mouse plasma (Biochemed 029-APSC-MP) were mixed with the N-Ac-Cys-AMA-10 and MPS to make a final concentration of N-Ac-Cys-AMA-10 and methyl phenyl sulfone 300 μM. The resulting plasma mixtures were incubated in a water bath at 37° C. Aliquots were taken before the reaction and on 1 day, 2 days, 4 days, and 7 days after the reaction, wherein each aliquot was 200 μL. To complete the reaction, a two-fold volume of acetonitrile was added, followed by brief vortexing, and centrifugation for plasma protein precipitation. Each supernatant obtained after centrifugation was collected and analyzed by HPLC. The compounds were detected and quantitated in the mouse and human plasma for up to 7 days, as indicated in FIG. 4. Studies demonstrate high stability of N-Ac-Cys-AMA-10 in plasma over a course of 7 days.

### Example 65. Relative Reaction Rates of ‘Reference A’, PyrMPS-1, and mMPS-4 with N-Ac-Cysteine

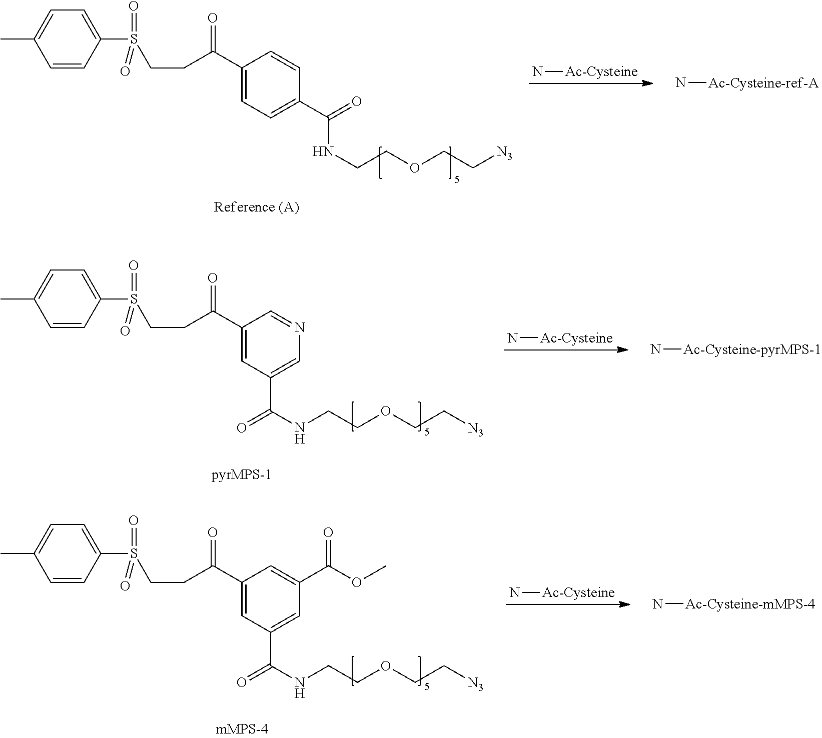

To a homogeneous solutions of ‘reference A’, PyrMPS-1, and mMPS-4 (10 stock solution 10 mmol) in PBS buffer (870 μL) and DMSO (90 μL) was added N-acetyl-L-cysteine (10 stock solution 10 mmol) and stirred for 1 hour at room temperature. The resulting samples were checked for reaction rate using LC/MS.

| Compound Name | Relative reaction rate |
|---|---|
| (Reference A) | 1 |
| pyrMSP-1 | Increase 11.5 folds |
| mMPS-4 | Increase 7 folds |

Introduction of the meta-substituents improves solubility of the compounds and affords a rapid reaction with a thiol group.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound of formula (I): 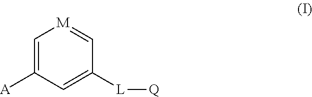
or a salt thereof, wherein: 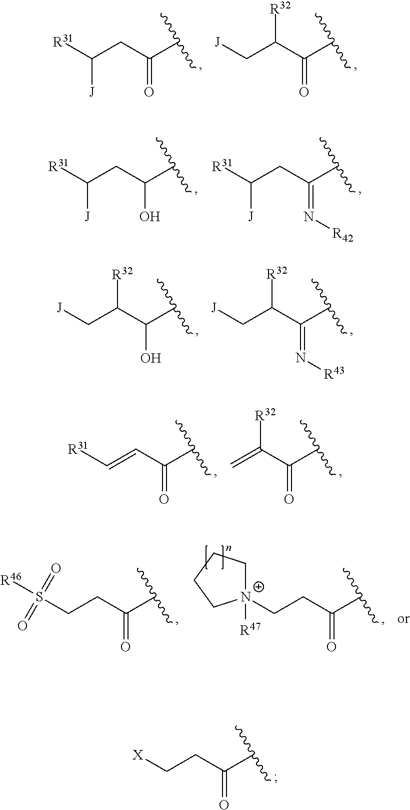
A is
M is N, CR³⁰, or C(-L-Q);
each L is independently selected from a spacer moiety;
each Q is independently selected from an active moiety or a reactive group;
X is selected from —Cl, —Br, and —I;
J is a targeting moiety;
R³⁰ and R³¹ are each independently selected from an electron-withdrawing group, hydrogen, alkyl, alkenyl, alkynyl, aryl, heteroaryl, carbocyclyl, heterocyclyl, and haloalkyl;
R⁴⁶ is selected from alkyl, alkenyl, alkynyl, aryl, heteroaryl, carbocyclyl, heterocyclyl, and haloalkyl;
R⁴² and R⁴³ are each independently selected from —OH, alkoxy, —NR⁴⁴R⁴⁵, alkyl, alkenyl, alkynyl, aryl, heteroaryl, carbocyclyl, and heterocyclyl, wherein R⁴⁴ and R⁴⁵ together with the nitrogen atom to which they are attached can form a 5-8-membered cycle, optionally fused with an aryl or a heteroaryl ring;
R³², R⁴⁴, and R⁴⁵ are each independently selected from hydrogen, alkyl, alkenyl, alkynyl, aryl, heteroaryl, carbocyclyl, heterocyclyl, and haloalkyl;
R⁴⁷ is O⁻, alkyl, alkenyl, alkynyl, aryl, heteroaryl, carbocyclyl, heterocyclyl; and n is 1 to 4.

2. The compound of claim 1, wherein M is N.

3. The compound of any one of the preceding claims, wherein M is CR³⁰, and wherein R³⁰ is an electron-withdrawing group.

4. The compound of any one of the preceding claims, wherein A is selected from 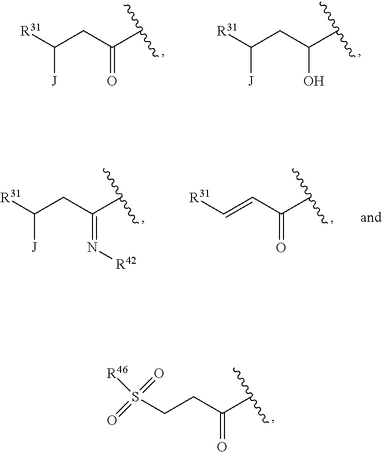
and wherein R³¹ is an electron-withdrawing group.

5. The compound of any one of the preceding claims, wherein M is C(-L-Q), and wherein L is coupled to C by an electron-withdrawing group, preferably wherein L is coupled to C by an electron-withdrawing group selected from an amide or an ester.

6. The compound of any one of the preceding claims, wherein R³⁰ is —CONR^{×}R³⁴ or —CO₂R³⁵, and R″, R³⁴, and R³⁵ are each independently selected from hydrogen, alkyl, alkenyl, alkynyl, aryl, heteroaryl, carbocyclyl, heterocyclyl, and haloalkyl.

7. The compound of any one of the preceding claims, wherein:
each electron-withdrawing group is independently selected from —NO₂, —CN, -haloalkyl, —CONR³³R³⁴, —CO₂R³⁵, —C(═O)R³⁶, —S(═O)R³⁷, —S(═O)₂OR³⁸, and —NR³⁹R⁴⁰R⁴¹; and
R³⁶, R³⁷, R³⁸ , R³⁹ , R⁴⁰, and R⁴¹ are each independently selected from hydrogen, alkyl, alkenyl, alkynyl, aryl, heteroaryl, carbocyclyl, heterocyclyl, and haloalkyl.

8. The compound of claim 7, wherein each electron-withdrawing group is independently selected from —CN, —CONR³³R³⁴, and —CO₂R³⁵.

9. The compound of claim 8, wherein each electron-withdrawing group is independently selected from —CN, —CONH₂, and —CO₂Me.

10. The compound of any of the preceding claims, wherein Q is an active moiety.

11. The compound of any one of the preceding claims, wherein Q comprises L′ and Q′, wherein L′ is a linker and Q′ is an active agent.

12. The compound of any one of the preceding claims, wherein L′ comprises a coupling group, wherein the coupling group is coupled to L.

13. The compound of claim 12, wherein the coupling group is selected from —C(═O)NR³²—, —C(═O)O—, —C(═NR³²)—, —C═NO—, —NR³²—C(═O)—NR³²—, —OC(═O)O—, —S— S—, —NR³²S(═O)₂O—, and —OS(═O)₂O—.

14. The compound of claim 12, wherein the coupling group is selected from

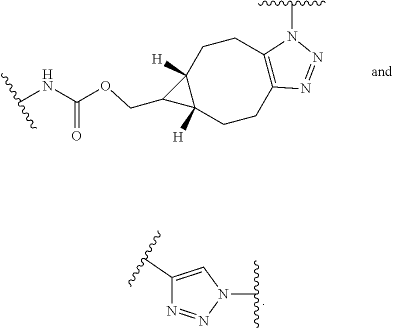 [SMILES: *NC(=O)OCC1[C@H]2CCc3nnn(*)c3CC[C@@H]12 ; *c1cn(*)nn1]

15. The compound of any one of the preceding claims, wherein L′ further comprises a cleavable group, wherein the cleavable group is coupled to Q′.

16. The compound of claim 15, wherein the cleavable group coupled to Q′ is selected from 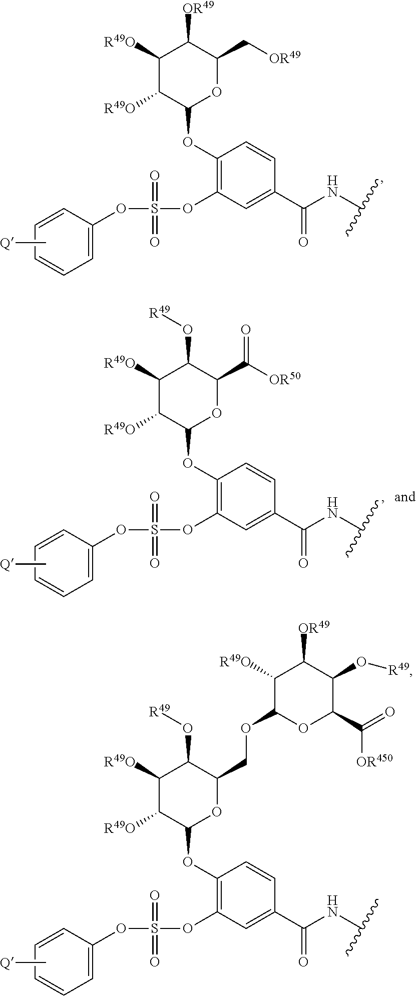
wherein
R⁴⁹ is hydrogen or —C(═O)R⁵⁰; and
R⁵⁰ is lower alkyl.

17. The compound of any one of the preceding claims, wherein L′ further comprises a C₆-C₁₀₀ alkylene comprising at least one group selected from —NH—, —C(═O)—, —O—, —S—, —S(O)—, and —S(═O)₂—.

18. The compound of any one of the preceding claims, wherein L comprises a C₆-C₁₀₀ alkylene comprising at least one group selected from —NH—, —C(═O)—, —O—, —S—, —S(O)—, and —S(═O)₂—.

19. The compound of any one of the preceding claims, wherein L comprises 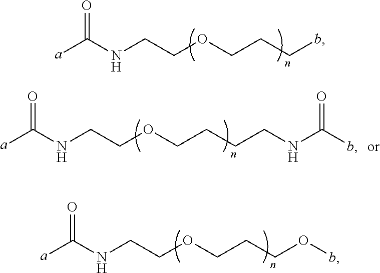
wherein
a is the bond to the M-containing aromatic ring, and b is the bond to L′; and
n is 2-20.

20. The compound of any one of the preceding claims, wherein Q′ is a hormone, an oligonucleotide, a toxin, an affinity ligand, a probe for detection, or a combination thereof.

21. The compound of any one of the preceding claims, wherein Q′ is selected from a cytokine, an immunomodulatory compound, an anti-cancer agent, an anti-viral agent, an anti-bacterial agent, an anti-fungal agent, an anthelmintic agent, or a combination thereof.

22. The compound of any one of claims 1-9, wherein Q is a reactive group.

23. The compound of claim 22, wherein the reactive group is selected from —N₃, —C═CH, 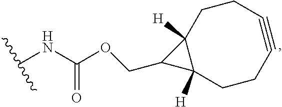 [SMILES: *NC(=O)OCC1[C@H]2CCC#CCC[C@@H]12]
—S(O)₂Hal, —NH₂, —CO₂Hal, —OH, —C(O)H, —SH, —N═C═O, and —N═S═C, wherein Hal is —Cl, —Br, or —I.

24. The compound of any one of the preceding claims, wherein the targeting moiety comprises an —S— moiety.

25. The compound of claim 24, wherein the targeting moiety is coupled to the remainder of the compound of formula (I) through the —S— moiety.

26. The compound of any one of claims 1-23, wherein A is

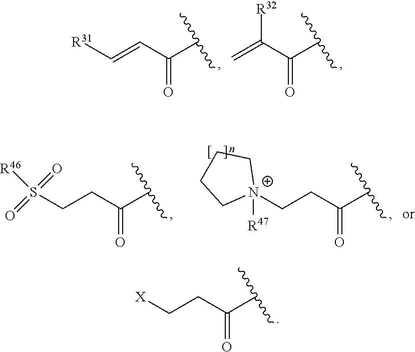

27. The compound of claim 26, wherein A is

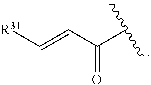

28. The compound of claim 27, wherein R³¹ is —CN, —CO₂NR³³R³⁴, or —CO₂R³⁵.

29. The compound of claim 26, wherein A is

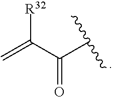

30. The compound of claim 29, wherein R³² is hydrogen or C₁₋₃ alkyl.

31. The compound of claim 26, wherein A is

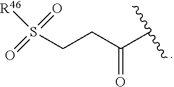

32. The compound of claim 31, wherein R⁴⁶ is optionally substituted C₁₋₃ alkyl, optionally substituted C₆-C₁₂ aryl, or optionally substituted heteroaryl.

33. The compound of claim 26, wherein A is

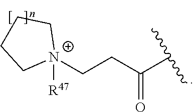

34. The compound of claim 33, wherein R⁴⁷ is O⁻ or C₁₋₃ alkyl.

35. The compound of claim 26, wherein A is

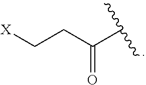

36. The compound of any one of claims 1-26, wherein the compound is selected
from

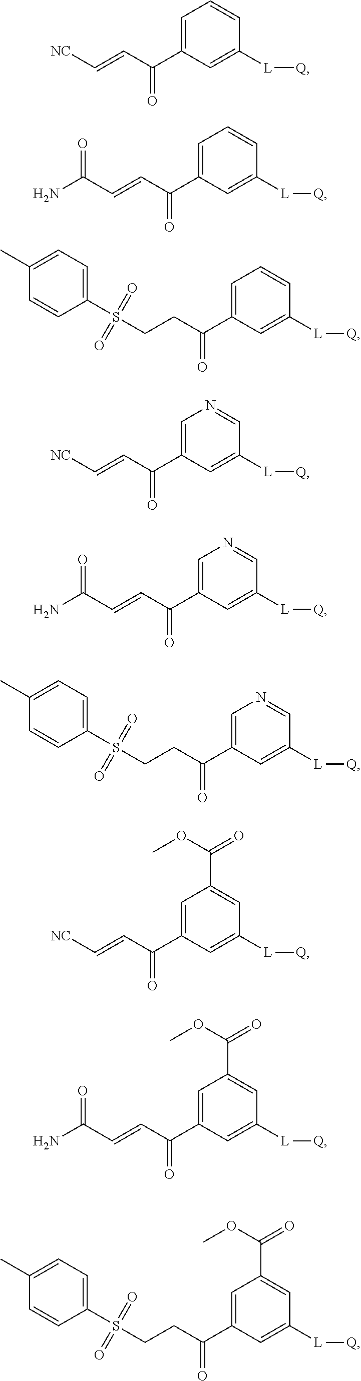

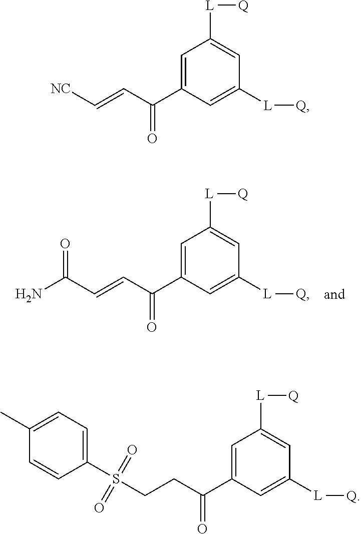

37. The compound of any one of claims 1-25, wherein A is

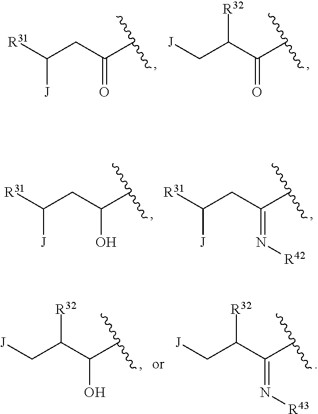

38. The compound of claim 37, wherein A is

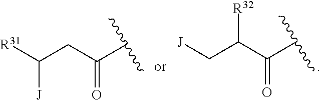

39. The compound of claim 37, wherein A is

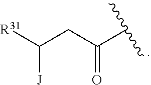

40. The compound of claim 37, wherein A is

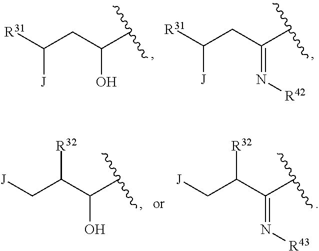

41. The compound of claim 40, wherein A is

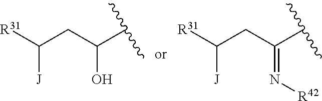

42. The compound of claim 41, wherein R⁴² is —OH or —NR⁴⁴R⁴⁵.

43. The compound of any one of claims 37-42, wherein the targeting moiety comprises a nanoparticle, an immunoglobulin, a nucleic acid, a protein, an oligopeptide, a polypeptide, an antibody, a fragment of an antigenic polypeptide, or a repebody.

44. The compound of claim 43, wherein the targeting moiety comprises an antibody, such as an antibody selected from an intact polyclonal antibody, an intact monoclonal antibody, an antibody fragment, a single chain Fv (scFv) mutant, a multispecific antibody, a bispecific antibody, a chimeric antibody, a humanized antibody, a human antibody, a fusion protein comprising an antigenic determinant portion of an antibody, and modified immunoglobulin molecules comprising antigen recognition sites.

45. The compound of claim 44, wherein the targeting moiety comprises an antibody selected from Muromonab-CD₃, Abciximab, Rituximab, Daclizumab, Palivizumab, Infliximab, Trastuzumab (herceptin), Etanercept, Basiliximab, Gemtuzumab ozogamicin, Alemtuzumab, Ibritumomab tiuxetan, Adalimumab, Alefacept, Omalizumab, Efalizumab, Tositumomob-I¹³¹, Cetuximab, Bevacizumab, Natalizumab, Ranibizumab, Panitumumab, Eculizumab, Rilonacept, Certolizumab pegol, Romiplostim, AMG-531, CNTO-148, CNTO-1275, ABT-874, LEA-29Y, Belimumab, TACI-Ig, Second generation anti-CD₂₀, ACZ-885, Tocilizumab, Atlizumab, Mepolizumab, Pertuzumab, Humax CD₂₀, Tremelimumab (CP-675 206), Ticilimumab, MDX-010, IDEC-114, Inotuzumab ozogamycin, HuMax EGFR, Aflibercept, HuMax-CD₄, Ala-Ala, ChAglyCD3, TRX4, Catumaxomab, IGN101, MT-201, Pregovomab, CH-14.18, WX-G250, AMG-162, AAB-001, Motavizumab, MEDI-524, Efumgumab, Aurograb, Raxibacumab, Third generation anti-CD₂₀, LY2469298, and Veltuzumab.

46. A method of preparing a conjugate, comprising reacting the compound of any one of claims 26-36 with a reagent comprising a targeting moiety covalently bound to a Michael donor, thereby producing a Michael adduct.

47. The method of claim 46, further comprising reducing the Michael adduct, thereby producing a compound of any one of claims 40-42.

48. The method of claim 46 or 47, wherein the Michael donor covalently bound to the targeting moiety is selected from:
—SH, —NH₂, —OH, 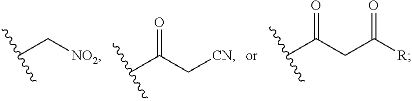
wherein R is C₁₋₃ alkyl or C₁₋₃ alkoxy.

49. The method of any one of claims 46-48, wherien the targeting moiety comprises a nanoparticle, an immunoglobulin, a nucleic acid, a protein, an oligopeptide, a polypeptide, an antibody, a fragment of an antigenic polypeptide, or a repebody.

50. The method of claim 49, wherein the targeting moiety comprises an antibody selected from an intact polyclonal antibody, an intact monoclonal antibody, an antibody fragment, a single chain Fv (scFv) mutant, a multispecific antibody, a bispecific antibody, a chimeric antibody, a humanized antibody, a human antibody, a fusion protein comprising an antigenic determinant portion of an antibody, and modified immunoglobulin molecules comprising antigen recognition sites.

51. The method of claim 50, wherein the targeting moiety comprises an antibody selected from Muromonab-CD₃, Abciximab, Rituximab, Daclizumab, Palivizumab, Infliximab, Trastuzumab (herceptin), Etanercept, Basiliximab, Gemtuzumab ozogamicin, Alemtuzumab, Ibritumomab tiuxetan, Adalimumab, Alefacept, Omalizumab, Efalizumab, Tositumomob-I¹³¹, Cetuximab, Bevacizumab, Natalizumab, Ranibizumab, Panitumumab, Eculizumab, Rilonacept, Certolizumab pegol, Romiplostim, AMG-531, CNTO-148, CNTO-1275, ABT-874, LEA-29Y, Belimumab, TACI-Ig, Second generation anti-CD₂₀, ACZ-885, Tocilizumab, Atlizumab, Mepolizumab, Pertuzumab, Humax CD₂₀, Tremelimumab (CP-675 206), Ticilimumab, MDX-010, IDEC-114, Inotuzumab ozogamycin, HuMax EGFR, Aflibercept, HuMax-CD₄, Ala-Ala, ChAglyCD3, TRX4, Catumaxomab, IGN101, MT-201, Pregovomab, CH-14.18, WX-G250, AMG-162, AAB-001, Motavizumab, MEDI-524, Efumgumab, Aurograb, Raxibacumab, Third generation anti-CD₂₀, LY2469298, and Veltuzumab.

52. A pharmaceutical composition comprising a compound of any one of claims 37-45 or a pharmaceutically acceptable salt thereof, and a pharmaceutically acceptable excipient.

53. A method for treating a disease or disorder, comprising administering a compound of any one of claims 37-45 or a pharmaceutically acceptable salt thereof, or a composition of claim 52 to a subject in need thereof.

54. The method of claim 54, wherein the disease or disorder is selected from cancer, infectious disease, or autoimmune disease.

55. The method of claim 53, wherein the disease or disorder is cancer.
